SEQUENCE LISTING (93 sequences)

SEQ ID NO: 1 - protein, 13 aa, Homo sapiens
CSARVGVGNTIYF

SEQ ID NO: 2 - protein, 13 aa, Homo sapiens
CSARSGVGNTIYF

SEQ ID NO: 3 - DNA, 43 bp, Homo sapiens
tgtgcctgtctctgtgcatctgtccatctgtgcgcatctgtgc

SEQ ID NO: 4 - protein, 14 aa, Homo sapiens
CARGAPLSIYGYTF

SEQ ID NO: 5 - protein, 15 aa, Homo sapiens
CASPPGRSTVNEQFF

SEQ ID NO: 6 - protein, 13 aa, Homo sapiens
CASSGQGGERQYF

SEQ ID NO: 7 - protein, 12 aa, Homo sapiens
CASSGWDNEQFF

SEQ ID NO: 8 - protein, 14 aa, Homo sapiens
CASSPTALGTEAFF

SEQ ID NO: 9 - protein, 17 aa, Homo sapiens
CASSRASTGLINYEQYF

SEQ ID NO: 10 - protein, 13 aa, Homo sapiens
CASSSRGLREQYF

SEQ ID NO: 11 - protein, 17 aa, Homo sapiens
CASSSRWGTGSGEEQYF

SEQ ID NO: 12 - protein, 16 aa, Homo sapiens
CASSTGEPGGYYEQYF

SEQ ID NO: 13 - protein, 16 aa, Homo sapiens
CATRTGAATSTDTQYF

SEQ ID NO: 14 - protein, 14 aa, Homo sapiens
CASRLAGVANEQFF

SEQ ID NO: 15 - protein, 14 aa, Homo sapiens
CASRLGTGDTEAFF

SEQ ID NO: 16 - protein, 14 aa, Homo sapiens
CASSLAGGNTEAFF

SEQ ID NO: 17 - protein, 16 aa, Homo sapiens
CASSLSGGGPYYGYTF

SEQ ID NO: 18 - protein, 13 aa, Homo sapiens
CASSLVADYGYTF

SEQ ID NO: 19 - protein, 16 aa, Homo sapiens
CASSPGGADSGNTIYF

SEQ ID NO: 20 - protein, 15 aa, Homo sapiens
CASSSRDRGLYEQYF

SEQ ID NO: 21 - protein, 13 aa, Homo sapiens
CASSWGFNTEAFF

SEQ ID NO: 22 - protein, 13 aa, Homo sapiens
CSARVGVGNTIYF

SEQ ID NO: 23 - protein, 13 aa, Homo sapiens
CASSFDGDTEAFF

SEQ ID NO: 24 - protein, 17 aa, Homo sapiens
CASSLGAGGRVNQPQHF

SEQ ID NO: 25 - protein, 17 aa, Homo sapiens
CAISEYPGQGAGQPQHF

SEQ ID NO: 26 - protein, 15 aa, Homo sapiens
CASRRTGGRKNTIYF

SEQ ID NO: 27 - protein, 13 aa, Homo sapiens
CASSDGLIYEQYF

SEQ ID NO: 28 - protein, 14 aa, Homo sapiens
CASSEAFWGSEQYF

SEQ ID NO: 29 - protein, 12 aa, Homo sapiens
CASSFLGQGEFF

SEQ ID NO: 30 - protein, 12 aa, Homo sapiens
CASSFPPIGQYF

SEQ ID NO: 31 - protein, 14 aa, Homo sapiens
CASSLAGLTYGYTF

SEQ ID NO: 32 - protein, 13 aa, Homo sapiens
CASSLDGFYGYTF

SEQ ID NO: 33 - protein, 15 aa, Homo sapiens
CASSLDSTGYNEQFF

SEQ ID NO: 34 - protein, 16 aa, Homo sapiens
CASSLGTGGTNTEAFF

SEQ ID NO: 35 - protein, 18 aa, Homo sapiens
CASSLKGAGAGVTDTQYF

SEQ ID NO: 36 - protein, 15 aa, Homo sapiens
CASSLSRDSQETQYF

SEQ ID NO: 37 - protein, 15 aa, Homo sapiens
CASSLVIQGSQPQHF

SEQ ID NO: 38 - protein, 12 aa, Homo sapiens
CASSLWQSEQYF

SEQ ID NO: 39 - protein, 12 aa, Homo sapiens
CASSPETDTQYF

SEQ ID NO: 40 - protein, 12 aa, Homo sapiens
CASSPETDTQYF

SEQ ID NO: 41 - protein, 14 aa, Homo sapiens
CASSPSYGGYEQYF

SEQ ID NO: 42 - protein, 17 aa, Homo sapiens
CASSQAMTEPLSYGYTF

SEQ ID NO: 43 - protein, 16 aa, Homo sapiens
CASSQDSGTPNYGYTF

SEQ ID NO: 44 - protein, 15 aa, Homo sapiens
CASSQETGSSYEQYF

SEQ ID NO: 45 - protein, 14 aa, Homo sapiens
CASSQGGHNQPQHF

SEQ ID NO: 46 - protein, 16 aa, Homo sapeins
CASSQGIDRVNQPQHF

SEQ ID NO: 47 - protein, 16 aa, Homo sapiens
CASSQGQASGANVLTF

SEQ ID NO: 48 - protein, 15 aa, Homo sapiens
CASSQRFGDSYEQYF

SEQ ID NO: 49 - protein, 17 aa, Homo sapiens
CASSQVGRGRNTGELFF

SEQ ID NO: 50 - protein, 16 aa, Homo sapiens
CASSSARDRIPSEAFF

SEQ ID NO: 51 - protein, 16 aa, Homo sapiens
CASSSEAGGRQETQYF

SEQ ID NO: 52 - protein, 13 aa, Homo sapiens
CASSSGQGQPQHF

SEQ ID NO: 53 - protein, 16 aa, Homo sapiens
CASSSPGQAGANVLTF

SEQ ID NO: 54 - protein, 13 aa, Homo sapiens
CASSSQLQEIQYF

SEQ ID NO: 55 - protein, 13 aa, Homo sapiens
CASSTDEPGELFF

SEQ ID NO: 56 - protein, 12 aa, Homo sapiens
CASSTVSYGYTF

SEQ ID NO: 57 - protein, 12 aa, Homo sapiens
CASSVTINVLTF

SEQ ID NO: 58 - protein, 14 aa, Homo sapiens
CASSWGQSGNTIYF

SEQ ID NO: 59 - protein, 14 aa, Homo sapiens
CASSYQKGRYEQYF

SEQ ID NO: 60 - protein, 16 aa, Homo sapiens
CASSYSDRDYYNEQFF

SEQ ID NO: 61 - protein, 14 aa, Homo sapiens
CASSYSMENTEAFF

SEQ ID NO: 62 - protein, 16 aa, Homo sapiens
CASSYSRSGGNTEAFF

SEQ ID NO: 63 - protein, 15 aa, Homo sapiens
CASSYSWREHNEQFF

SEQ ID NO: 64 - protein, 14 aa, Homo sapiens
CATSAGTGGSPLHF

SEQ ID NO: 65 - protein, 17 aa, Homo sapiens
CATSDGRLQGGSQPQHF

SEQ ID NO: 66 - protein, 14 aa, Homo sapiens
CATVAGLEGYEQYF

SEQ ID NO: 67 - protein, 16 aa, Homo sapiens
CSARDDKQGISTEAFF

SEQ ID NO: 68 - protein, 13 aa, Homo sapiens
CSARSGVGNTIYF

SEQ ID NO: 69 - protein, 13 aa, Homo sapiens
CSATRMFNQPQHF

SEQ ID NO: 70 - protein, 14 aa, Homo sapiens
CSVGTGGTNEKLFF

SEQ ID NO: 71 - protein, 14 aa, Homo sapiens
CSVPGEVTPGELFF

SEQ ID NO: 72 - protein, 15 aa, Homo sapiens
CSVVRIGVGQETQYF

SEQ ID NO: 73 - protein, 14 aa, Homo sapiens
CASSAGPKNQPQHF

SEQ ID NO: 74 - protein, 15 aa, Homo sapiens
CASSLKVGRGTEAFF

SEQ ID NO: 75 - protein, 15 aa, Homo sapiens
CASSLNPRGTDTQYF

SEQ ID NO: 76 - protein, 19 aa, Homo sapiens
CASSPPSPIRDRGPYGYTF

SEQ ID NO: 77 - protein, 12 aa, Homo sapiens
CASSSWAYGYTF

SEQ ID NO: 78 - protein, 14 aa, Homo sapiens
CASSWGASSYEQYF

SEQ ID NO: 79 - protein, 15 aa, Homo sapiens
CASSYSGVGGWEQYF

SEQ ID NO: 80 - protein, 16 aa, Homo sapiens
CSAIVGNGGGNQPQHF

SEQ ID NO: 81 - protein, 14 aa, Homo sapiens
CSARDMDGDYEQYF

SEQ ID NO: 82 - protein, 16 aa, Homo sapiens
CSVLVRDLANKNIQYF

SEQ ID NO: 83 - protein, 13 aa, Homo sapiens
CASSPARGVGQYF

SEQ ID NO: 84 - protein, 15 aa, Homo sapiens
CASSPTGAGDQPQHF

SEQ ID NO: 85 - protein, 19 aa, Homo sapiens
CASSQFWVRFSGNQETQYF

SEQ ID NO: 86 - protein, 15 aa, Homo sapiens
CASSSSGPGNQPQHF

SEQ ID NO: 87 - protein, 15 aa, Homo sapiens
CASSSTGGGDQPQHF

SEQ ID NO: 88 - protein, 12 aa, Homo sapiens
CAMPKLGETQYF

SEQ ID NO: 89 - protein, 13 aa, Homo sapiens
CASRKQGPYGYTF

SEQ ID NO: 90 - protein, 15 aa, Homo sapiens
CASSANQGAGAPLHF

SEQ ID NO: 91 - protein, 15 aa, Homo sapiens
CASSHKVNSGNTIYF

SEQ ID NO: 92 - protein, 14 aa, Homo sapiens
CASSIVWGAYEQYF

SEQ ID NO: 93 - protein, 15 aa, Homo sapiens
CASSPGQGEGYEQYF

(19) United States
(12) Patent Application Publication
(10) Pub. No.: US 2021/0155986 A1
(43) Pub. Date: May 27, 2021

(54) NON-INVASIVE DETECTION OF RESPONSE TO IMMUNOTHERAPY

(71) Applicant: The Johns Hopkins University, Baltimore, MD (US)

(72) Inventors: Victor E. Velculescu, Dayton, MD (US); Valsamo Anagnostou, Baltimore, MD (US)

(21) Appl. No.: 17/047,006

(22) PCT Filed: Apr. 12, 2019
(86) PCT No.: PCT/US2019/027213

§ 371 Date: Oct. 12, 2020

Related U.S. Application Data
(60) Provisional application No. 62/657,600, filed on Apr. 13, 2018.

(51) Int. Cl. C12Q 1/6876; A61K 45/06

(52) U.S. Cl. CPC C12Q 1/6876; C12Q 2600/106; A61K 45/06

(57) ABSTRACT

Provided herein are method of determining the efficacy of an immunotherapy in a subject by detecting changes in levels of circulating tumor DNA (ctDNA) and/or differences in TCR clonotype levels. Also provided herein are method of determining resistance to an immunotherapy in a subject by detecting changes in levels of circulating tumor DNA (ctDNA) and/or differences in TCR clonotype levels.

### STATEMENT AS TO FEDERALLY SPONSORED RESEARCH

This invention was made with government support under grant numbers NIH RO1 grant (CA121113) and UL1TR001079 awarded by the National Institutes of Health. The government has certain rights in the invention.

### TECHNICAL FIELD

The present disclosure relates generally to the field of cancer. More specifically, this disclosure relates to non-invasive in vitro methods for determining the efficacy of cancer immunotherapy.

### BACKGROUND

Despite the durable clinical benefit observed with immune checkpoint inhibitors for cancer patients (e.g., non-small cell lung cancer (NSCLC) patients), the majority of patients either do not benefit from therapy or develop acquired resistance after an initial response. The plasticity of the immune system under immunotherapy has weakened single biomarker-driven approaches and currently used predictive biomarkers have been unable to accurately define which subset of patients will benefit from these therapies.

### SUMMARY

In one aspect, provided herein are methods of predicting the efficacy of an immunotherapy in a subject having been previously diagnosed with cancer and having received at least one dose of an immunotherapy.

In some embodiments, provided herein are methods of determining the efficacy of an immunotherapy in a subject that include: detecting a first level of circulating tumor DNA (ctDNA) and a first level of at least one TCR clonotype in a biological sample isolated from the subject at a first time point, detecting a second level of ctDNA and a second level of the at least one TCR clonotype in a biological sample obtained from the subject at a second time point, wherein the subject has received at least one dose of an immunotherapy between the first time point and the second time point, and identifying the immunotherapy as being effective in a subject having: (i) a reduced second level of ctDNA as compared to the first level of ctDNA, and (ii) an increased second level of the at least one TCR clonotype as compared to the first level of the at least one TCR clonotype. In some embodiments, detecting and comparing both ctDNA levels and TCR clonotype levels at different time points is superior in determining the efficacy of an immunotherapy as compared to detecting and comparing either ctDNA levels or TCR clonotype levels individually.

In some embodiments, provided herein are methods of determining the efficacy of immunotherapy in a subject that include: detecting a first level of circulating tumor DNA (ctDNA) in a biological sample isolated from the subject at a first time point, detecting a second level of ctDNA in a biological sample obtained from the subject at a second time point, wherein the subject has received at least one dose of an immunotherapy between the first time point and the second time point, and identifying the immunotherapy as being effective in a subject having a reduced second level of ctDNA as compared to the first level of ctDNA. In some embodiments of methods of determining the efficacy of immunotherapy in a subject, the methods further include detecting a first level of at least one TCR clonotype in the biological sample obtained from the subject at the first time point, detecting a second level of at least one TCR clonotype in the biological sample obtained from the subject at the second time point, and identifying the immunotherapy as being effective in a subject having a reduced second level of ctDNA as compared to the first level of ctDNA and an increased second level of the at least one TCR clonotype as compared to the first level of the at least one TCR clonotype.

In some embodiments, provided herein are methods determining the efficacy of immunotherapy in a subject that include: detecting a first level of at least one TCR clonotype in the biological sample obtained from the subject at the first time point, detecting a second level of at least one TCR clonotype in the biological sample obtained from the subject at the second time point; and identifying the immunotherapy as being effective in a subject having an increased second level of the at least one TCR clonotype as compared to the first level of the at least one TCR clonotype.

In some embodiments of any of the methods disclosed herein, a biological sample obtained from the subject at the first time point, the second time point, or both comprises blood, plasma, serum, urine, cerebrospinal fluid, saliva, sputum, broncho-alveolar lavage, bile, lymphatic fluid, cyst fluid, stool, uterine lavage, vaginal fluids, ascites, and combinations thereof. In some embodiments of any of the methods disclosed herein, a step of detecting includes using a method selected from the group consisting of: a targeted capture method, a next-generation sequencing method, an array-based method, and combinations thereof.

In some embodiments of any of the methods disclosed herein in which ctDNA is detected, the step of detecting (e.g., a first level of ctDNA, a second level of ctDNA, or both) includes: extracting cell-free DNA from blood, ligating a low complexity pool of dual index barcode adapters to the cell-free DNA to generate a plurality of barcode adapter-ligated cell-free DNA segments, capturing the plurality of barcode adapter-ligated cell-free DNA segments; sequencing the plurality of captured barcode adapter-ligated cell-free DNA segments;

aligning the sequenced plurality of captured barcode adapter-ligated cell-free DNA segments to a reference genome, and identifying sequence alterations using aligned sequences of multiple distinct molecules containing identical redundant changes.

In some embodiments of any of the methods disclosed herein in which ctDNA is detected (e.g., at a first time point, a second time point, or both), a second level of ctDNA is at least about 2-fold lower than the first level of ctDNA. In some embodiments of any of the methods disclosed herein in which at least one TCR clonotype is detected (e.g., at a first time point, a second time point, or both), the second level of the at least one TCR clonotype is at least about 2-fold higher than the first level of the at least one TCR clonotype.

In some embodiments of any of the methods disclosed herein, an immunotherapy is selected from the group consisting of: an antibody, an adoptive cell therapy, a chimeric antigen receptor (CAR) T cell therapy, an antibody-drug conjugate, a cytokine therapy, a cancer vaccine, a checkpoint inhibitor, and combinations thereof. In some embodiments, the immunotherapy comprises a checkpoint inhibitor. In some embodiments, the checkpoint inhibitor is selected from the group consisting of: a CTLA-4 inhibitor, a PD-1 inhibitor, a PD-L1 inhibitor, and combinations thereof. In some embodiments, the checkpoint includes a CTLA-4 inhibitor.

In some embodiments of any of the methods disclosed herein, the subject has been previously administered a different treatment or immunotherapy and the different treatment or immunotherapy was determined not to be therapeutically effective. In some embodiments of any of the methods disclosed herein, one or more additional doses of an immunotherapy identified as being effective is administered to the subject. In some embodiments of any of the methods disclosed herein, a therapeutic intervention is administered to the subject. In some embodiments, the therapeutic intervention is selected from the group consisting of: a different immunotherapy, an antibody, a chimeric antigen receptor (CAR) T cell therapy, an adoptive T cell therapy, an antibody-drug conjugate, a cytokine therapy, a cancer vaccine, a checkpoint inhibitor, radiation therapy, surgery, a chemotherapeutic agent, and combinations thereof.

In some embodiments of any of the methods disclosed herein, the subject has cancer. In some embodiments, the cancer is selected from the group consisting of: a head and neck cancer, a central nervous system cancer, a lung cancer, a mesothelioma, an esophageal cancer, a gastric cancer, a gall bladder cancer, a liver cancer, a pancreatic cancer, a melanoma, an ovarian cancer, a small intestine cancer, a colorectal cancer, a breast cancer, a sarcoma, a kidney cancer, a bladder cancer, an uterine cancer, a cervical cancer, and a prostate cancer.

In some embodiments, provided herein are methods of determining resistance to an immunotherapy in a subject having cancer that include: detecting a first level circulating tumor DNA (ctDNA) in a biological sample isolated from the subject at a first time point, detecting a second level of ctDNA in a biological sample obtained from the subject at a second time point, wherein the subject has received at least one dose of an immunotherapy between the first time point and the second time point, and identifying the subject as having resistance when the second level of ctDNA is not substantially reduced as compared to the first level of ctDNA. In some embodiments of methods of determining resistance to an immunotherapy in a subject, the methods further include detecting the level of at least one TCR clonotype in the biological sample obtained from the subject at the first time point, detecting a second level of at least one TCR clonotype in the biological sample obtained from the subject and at the second time point, and identifying the subject as having resistance when the second level of ctDNA is not substantially reduced as compared to the first level of ctDNA and when the second level of the at least one TCR clonotype is not substantially increased as compared to the first level of the at least one TCR clonotype.

In some embodiments, provided herein are methods of determining resistance to an immunotherapy in a subject having cancer that include: detecting the level of at least one TCR clonotype in the biological sample obtained from the subject at the first time point, detecting a second level of at least one TCR clonotype in the biological sample obtained from the subject and at the second time point, and identifying the subject as having resistance when the second level of the at least one TCR clonotype is not substantially increased as compared to the first level of the at least one TCR clonotype.

In some embodiments, provided herein are methods of determining poor efficacy of an immunotherapy in a subject having cancer that include: detecting a first level circulating tumor DNA (ctDNA) in a biological sample isolated from the subject at a first time point, detecting a second level of ctDNA in a biological sample obtained from the subject at a second time point, wherein the subject has received at least one dose of an immunotherapy between the first time point and the second time point, and identifying the immunotherapy as having poor efficacy when the second level of ctDNA is not substantially reduced as compared to the first level of ctDNA. In some embodiments, the second level of ctDNA is at least about 2-fold higher than the first level of ctDNA. In some embodiments of determining poor efficacy of an immunotherapy in a subject having cancer, the method further includes: detecting a first level of at least one TCR clonotype in the biological sample obtained from the subject at the first time point, detecting a second level of at least one TCR clonotype in the biological sample obtained from the subject at the second time point, and identifying the immunotherapy as having poor efficacy in a subject when the second level of ctDNA is not substantially reduced as compared to the first level of ctDNA and when the second level of the at least one TCR clonotype is not substantially increased as compared to the first level of the at least one TCR clonotype. In some embodiments, a subject is identified as having poor prognosis when the immunotherapy was identified as having poor efficacy. In some embodiments, the poor prognosis is selected from the group consisting of: shorter progression-free survival, lower overall survival, and combinations thereof. In some embodiments, the immunotherapy comprises a checkpoint inhibitor. In some embodiments, the checkpoint inhibitor is selected from the group consisting of: a CTLA-4 inhibitor, a PD-1 inhibitor, a PD-L1 inhibitor, and combinations thereof. In some embodiments of determining poor efficacy of an immunotherapy in a subject having cancer, the method further includes administering a therapeutic intervention to the subject, wherein the therapeutic intervention is not the immunotherapy. In some embodiments, the therapeutic intervention is selected from: a different immunotherapy, an antibody, adoptive T cell therapy, a chimeric antigen receptor (CAR) T cell therapy, an antibody-drug conjugate, a cytokine therapy, a cancer vaccine, a checkpoint inhibitor, radiation therapy, surgery, a chemotherapeutic agent, and combinations thereof.

In some embodiments of determining resistance to an immunotherapy in a subject having cancer or determining poor efficacy of an immunotherapy in a subject having cancer in which ctDNA is detected, the step of detecting (e.g., a first level of ctDNA, a second level of ctDNA, or both) includes: extracting cell-free DNA from blood, ligating a low complexity pool of dual index barcode adapters to the cell-free DNA to generate a plurality of barcode adapter-ligated cell-free DNA segments, capturing the plurality of barcode adapter-ligated cell-free DNA segments, sequencing the plurality of captured barcode adapter-ligated cell-free DNA segments; aligning the sequenced plurality of captured barcode adapter-ligated cell-free DNA segments to a reference genome and identifying sequence alterations using aligned sequences of multiple distinct molecules containing identical redundant changes.

In some embodiments of any of the methods disclosed herein, the second time point is about two to about six weeks after the first time point. In some embodiments of any of the methods disclosed herein, the second time point is about four weeks after the first time point.

Skilled practitioners will appreciate that a subject can be diagnosed, e.g., by a medical professional, e.g., a physician or nurse (or veterinarian, as appropriate for the subject being diagnosed), as suffering from or at risk for a condition described herein, e.g., cancer, using any method known in the art, e.g., by assessing a subject's medical history, performing diagnostic tests, and/or by employing imaging techniques.

Skilled practitioners will also appreciate that treatment need not be administered to a subject by the same individual who diagnosed the subject (or the same individual who prescribed the treatment for the subject). Treatment can be administered (and/or administration can be supervised), e.g., by the diagnosing and/or prescribing individual, and/or any other individual, including the subject her/himself (e.g., where the subject is capable of self-administration).

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

FIG. 1 is a schematic overview of next-generation sequencing and T cell analyses. Serial blood samples were collected at baseline, early after treatment initiation and at additional time points during immune checkpoint blockade to determine ctDNA and TCR repertoire dynamics. ctDNA trends were evaluated by TEC-Seq and the evolving TCR repertoire was assessed by TCR next generation sequencing. Dynamic changes in ctDNA and TCR clonotypic expansions were used to identify molecular response patterns and compared to RECIST 1.1 tumor burden evaluations.

FIG. 2A is a line graph showing ctDNA changes for a patient with sustained response to anti-PD1 therapy.

FIG. 2B shows representative computerized tomography (CT) images taken at baseline, 9 weeks and 30 weeks following anti-PD1 therapy according to FIG. 2A.

FIG. 2C is a line graph showing ctDNA changes for a patient with acquired resistance to anti-PD1 therapy.

FIG. 2D shows representative CT images taken at baseline, 7 weeks and 15 weeks following anti-PD1 therapy according to FIG. 2C.

FIG. 2E is a line graph showing ctDNA changes for a patient with primary resistance to anti-PD1 therapy.

FIG. 2F shows representative CT images taken at baseline and 5 weeks following anti-PD1 therapy according to FIG. 2E.

FIG. 3A is a line graph showing ctDNA changes for a patient with sustained response to anti-PD1 therapy at 4 weeks.

FIG. 3B is a graph showing capture rate of CT imaging at baseline, week 4 and week 9 after anti-PD1 therapy.

FIG. 3C shows representative CT images taken at baseline, 9 weeks and 30 weeks.

FIG. 3D is a line graph showing individual TCR clone expansion over time.

FIG. 3E is a line graph showing average productive frequencies of TCR clones over time.

FIG. 4A is a line graph showing ctDNA changes for a patient with primary resistance to anti-PD1 therapy.

FIG. 4B is a graph showing capture rate of CT imaging at baseline, week 4 and week 5 after anti-PD1 therapy.

FIG. 4C shows representative CT images taken at baseline and 5 weeks.

FIG. 4D is a line graph showing individual TCR clone expansion over time.

FIG. 4E is a line graph showing average productive frequencies of TCR clones over time.

FIG. 5A is a line graph showing ctDNA clonal dynamics during anti-PD1 treatment for patient CGLU168.

FIG. 5B is a line graph showing ctDNA clonal dynamics during anti-PD1 treatment for patient CGLU160.

FIG. 5C is a line graph showing ctDNA clonal dynamics during anti-PD1 treatment for patient CGLU117.

FIG. 5D is a line graph showing ctDNA clonal dynamics during anti-PD1 treatment for patient CGLU211.

FIG. 5E is a line graph showing ctDNA clonal dynamics during anti-PD1 treatment for patient CGLU212.

FIG. 5F is a line graph showing ctDNA clonal dynamics during anti-PD1 treatment for patient CGLU135.

FIG. 6A is a graph showing probability of survival over time in patients with reduction of ctDNA levels to undetectable levels.

FIG. 6B is a graph showing probability of survival over time in patients with differential response to anti-PD1 therapy as compared to tumor mutation burden (TMB).

FIG. 6C is a graph showing overall survival over time in patients with reduction of ctDNA levels to undetectable levels.

FIG. 6D is a graph showing overall survival over time in patients with differential response to anti-PD1 therapy as compared to TMB.

FIG. 7A is a representative line graph showing intratumoral TCR clonotypic amplifications over time in peripheral blood from patient CGLU127 at baseline, 18 weeks and 30 weeks after PD1-therapy.

FIG. 7B is a representative line graph showing productive frequencies of intratumoral clones in the peripheral blood from patient CGLU161 at the time of acquired resistance compared to radiographic response.

FIG. 7C is a representative line graph showing productive frequencies of intratumoral clones in the peripheral blood from patient CGLU135 at the time of acquired resistance compared to radiographic response.

FIG. 7D is a representative line graph showing intratumoral TCR clonotypic amplifications over time in peripheral blood from patient CGLU117 at baseline, 18 weeks and 30 weeks after PD1-therapy.

FIG. 8A is a representative line graph showing intratumoral TCR clonotypic amplifications over time in peripheral blood from patient CGLU115 at baseline, 18 weeks and 30 weeks after PD1-therapy.

FIG. 8B is a representative line graph showing intratumoral TCR clonotypic amplifications over time in peripheral blood from patient CGLU159 between baseline and week 4 after PD1-therapy.

FIG. 8C is a representative line graph showing intratumoral TCR clonotypic amplifications over time in peripheral blood from patient CGLU162 at baseline, week 4, week 11 and week 16 after PD1-therapy.

FIG. 9 is a graph showing CDR3 length distribution among intratumoral TCR clones in pre-treatment peripheral blood.

FIG. 10 is a bar graph showing differential VJ gene usage for patient CGLU111 between baseline and the time of radiographic response (week 18). Clones with significant expansions are colored; TCR clonotypes with no significant expansions between the two time points are shown in gray.

FIG. 11 is a bar graph showing differential VJ gene usage for patient CGLU127 between baseline and the time of radiographic response (week 18). Clones with significant expansions are colored; TCR clonotypes with no significant expansions between the two time points are shown in gray.

FIG. 12 is a bar graph showing differential VJ gene usage for patient CGLU127 between baseline and the time of radiographic response (week 8). Clones with significant expansions are colored; TCR clonotypes with no significant expansions between the two time points are shown in gray.

FIG. 13 is a bar graph showing differential VJ gene usage for patient CGLU135 between time of response (week 44) and the time of acquired resistance (week 110). Clones with significant expansions are colored; TCR clonotypes with no significant expansions between the two time points are shown in gray.

FIG. 14 is a bar graph showing differential VJ gene usage for patient CGLU161 between time of response (week 26) and the time of acquired resistance (week 34). Clones with significant expansions are colored; TCR clonotypes with no significant expansions between the two time points are shown in gray.

FIG. 15 is a bar graph showing VJ gene usage for patient CGLU115. TCR clonotypes with no significant expansions between the two time points are shown in gray.

FIG. 16 is a bar graph showing differential VJ gene usage for patient CGLU121. TCR clonotypes with no significant expansions between the two time points are shown in gray.

FIG. 17 is a bar graph showing differential VJ gene usage for patient CGLU159 between baseline and week 11. Clones with significant expansions are colored; TCR clonotypes with no significant expansions between the two time points are shown in gray.

FIG. 18 is a bar graph showing differential VJ gene usage for patient CGLU162 between baseline and week 10. Clones with significant expansions are colored; TCR clonotypes with no significant expansions between the two time points are shown in gray.

### DETAILED DESCRIPTION

Provided herein are methods of non-invasive molecular analysis and evaluation of tumor-intrinsic (e.g., ctDNA) and tumor-extrinsic (e.g., TCR repertoire) parameters that are useful for rapidly predicting which subjects would ultimately benefit from immune checkpoint blockade. Such methods can be useful for immune targeted agents as the therapeutic response of these approaches has been challenging to evaluate using radiographic imaging due to tumor immune infiltration (3). Conventional response criteria such as the Response Evaluation Criteria in Solid Tumors (RECIST) suffer from various deficiencies in estimating the benefit from immunotherapies and may not capture the unique patterns and timing of anti-tumor immune responses (4, 5).

The temporal relationship between ctDNA detection and emergence of recurrent or progressive disease has been shown in patients with early stage NSCLC (6-8) and in advanced stage patients receiving targeted therapies (9). ctDNA changes have been associated with therapeutic outcome during immune checkpoint blockade in NSCLC. However, these analyses have been limited by the low sensitivity of the approaches, permitting analyses in approximately half of the cases analyzed. Even less is known about the dynamics of the peripheral T cell repertoire during immune checkpoint blockade in NSCLC and how these relate to ctDNA levels and tumor response. To overcome these issues and to allow ultrasensitive evaluation of ctDNA during therapy, a custom capture and sequencing approach, targeted error-correction sequencing (TEC-Seq), was developed that permits sensitive and specific detection of low abundance sequence alterations using next generation sequencing (8). Methods of evaluating TCR clonal expansion in the tumor microenvironment during immune checkpoint blockade have also been developed (14).

Considering the increased human and financial cost to both patients and health systems, it has become clear that success of immunotherapy approaches depends on choosing patient populations most likely to benefit. There is therefore an urgent clinical need to develop molecular assays of response and resistance to immune targeted agents.

There is now an appreciation of patients with a hyperprogressor clinical phenotype (26), suggesting potentially deleterious effects of immune checkpoint blockade in a fraction of treated individuals. For these patients, accurate and early prediction of treatment failure would be useful. In some embodiments of method provided herein, evaluation of ctDNA kinetics very early after treatment initiation allows subjects with hyper-progression to be rapidly identified and redirected to receive alternative options. For example, patient CGLU121 exhibited rapidly progressive disease, and also exhibited increase in ctDNA levels at week 4 after initiation of therapy predicted early tumor progression.

Clonal expansion of intra-tumoral T cells may predict therapeutic outcome for immune checkpoint blockade (27). However, little was previously known about the significance of peripheral expansion of TCR clones found in the tumor microenvironment during therapy. Expansion of peripheral CD8+ T cell populations has been shown to precede immune-related adverse events in patients treated with ipilimumab (28). While there were cases for which TCR expansion preceded the development of a grade 2-4 immune-related adverse events (see, e.g., CGLU161, CGLU117), such events were also noted significantly later from the time of TCR expansion (see, e.g., CGLU111, CGLU135). These observations are consistent with the notion that the expansion of peripheral TCRs reflect an anti-tumor immune response rather than autoimmune reactivity.

As used herein, the word “a” or “an” before a noun represents one or more of the particular noun. For example, the phrase “an immunotherapy” encompasses “one or more immunotherapies.”

As used herein, the term “about” means approximately, in the region of, roughly, or around. When used in conjunction with a numerical range, the term “about” modifies that range by extending the boundaries above and below the numerical values set forth. In general, the term “about” is used herein to modify a numerical value above and below the stated value by a variance of 10%.

As used herein, the term “subject” means a vertebrate, including any member of the class mammalia, including humans, domestic and farm animals, and zoo, sports or pet animals, such as mouse, rabbit, pig, sheep, goat, cattle, horse (e.g., race horse), and higher primates. In some embodiments, the subject is a human. In some embodiments, the subject is a human harboring a cancer cell.

The term “treat(ment)” is used herein to denote delaying the onset of, inhibiting, alleviating the effects or progression of, or prolonging the life of a patient suffering from, a condition, e.g., cancer.

The terms “effective amount” and “amount effective to treat” as used herein, refer to an amount or concentration of a composition or treatment described herein, e.g., an immunotherapy, utilized for a period of time (including acute or chronic administration and periodic or continuous administration) that is effective within the context of its administration for causing an intended effect or physiological outcome. For example, effective amounts of an immunotherapy (e.g., any immunotherapy described herein) for use in the present disclosure include, for example, amounts that inhibit the growth of cancer, e.g., tumors and/or tumor cells, improve, delay tumor growth, improve survival for a patient suffering from or at risk for cancer, and improve the outcome of other cancer treatments As another example, effective amounts of an immunotherapy (e.g., any immunotherapy described herein) can include amounts that advantageously affect a tumor microenvironment (e.g., increase the level of at least one TCR clonotype and TCR clonality) and reduce the levels of circulating tumor DNA (ctDNA) in a sample.

The terms “a reduced level” or a “decreased level” is a reduction or decrease in the level of a particular substance or particular substances (e.g., ctDNA) of at least about 2-fold (e.g., at least about 4-fold, at least about 6-fold, at least about 8-fold, at least about 10-fold, at least about 12-fold, at least about 14-fold, at least about 20-fold) as compared to a reference level or value. In some embodiments, a reduced level is a reduction of or decrease in a second level of a particular substance or particular substances of at least about 1% (e.g., at least about 2%, at least about 4%, at least about 6%, at least about 8%, at least about 10%, at least about 12%, at least about 14%, at least about 16%, at least about 18%, at least about 20%, at least about 22%, at least about 24%, at least about 26%, at least about 28%, at least about 30%, at least about 40%, at least about 45%, at least about 50%, at least about 60%, at least about 65%, at least about 70%, at least about 75%, at least about 80%, at least about 85%, at least about 90%, at least about 95%, or at least about 99%) as compared to the first level of the particular substance or particular substances.

The terms “an increased level” or a “higher level” is an increase of at least about 2-fold (e.g., at least about 4-fold, at least about 6-fold, at least about 8-fold, at least about 10-fold, at least about 12-fold, at least about 14-fold, at least about 20-fold, or more) of a particular substance or particular substances (e.g., at least one TCR clonotype). In some embodiments, an increased level of at least one TCR clonotype(s) (e.g., at least two, at least three, at least four, at least five, at least six, at least seven, at least eight, at least nine, at least ten, at least twelve, at least fifteen, at least twenty, or more clonotypes) is at least 2, 3, 4, 5, 6, 7, 8, 9, 10, 11, 12, 13, 14, 15, 16, 17, 18, 19, 20, 21, 22, 23, 24, 25, 26, 27, 28, 29, or 30-fold higher as compared to a first or reference level of the TCR clonotypes. In some embodiments, an increased level of at least one TCR clonotype(s) is an increase of at least about 1% (e.g., at least about 2%, at least about 4%, at least about 6%, at least about 8%, at least about 10%, at least about 12%, at least about 14%, at least about 16%, at least about 18%, at least about 20%, at least about 22%, at least about 24%, at least about 26%, at least about 28%, at least about 30%, at least about 40%, at least about 45%, at least about 50%, at least about 60%, at least about 65%, at least about 70%, at least about 75%, at least about 80%, at least about 85%, at least about 90%, at least about 95%, or at least about 99%) of a second level of at least one TCR clonotype (e.g., at least two, at least three, at least four, at least five, at least six, at least seven, at least eight, at least nine, at least ten, at least twelve, at least fifteen, at least twenty, or more clonotypes)) as compared to a first or reference level of at least one TCR clonotype(s).

The terms “not substantially reduced” or “not substantially decreased” refer to clinically insignificant changes (e.g., a reduction, decrease) in the second level of a particular substance or particular substances (e.g., ctDNA) as compared to the first level of the particular substance or particular substances. In some embodiments, a not substantially reduced second level of a particular substance or particular substances (e.g., ctDNA) is a reduction or decrease in levels of less than about 10% (e.g., less than about 9%, less than about 8%, less than about 7%, less than about 6%, less than about 5%, less than about 4%, less than about 3%, less than about 2%, less than about 1%, less than about 0.5%, less than about 0.25%, less than about 0.2%, less than about 0.1%, less than about 0.05%, less than about 0.01%) as compared to the first level of ctDNA. In some embodiments, a not substantially reduced level of a particular substance or particular substances (e.g., ctDNA) is an increase of at least about 0.5% (e.g., at least about 1%, at least about 2%, at least about 4%, at least about 6%, at least about 8%, at least about 10%, at least about 12%, at least about 14%, at least about 16%, at least about 18%, at least about 20%, at least about 22%, at least about 24%, at least about 26%, at least about 28%, at least about 30%, at least about 40%, at least about 45%, at least about 50%, at least about 60%, at least about 65%, at least about 70%, at least about 75%, at least about 80%, at least about 85%, at least about 90%, at least about 95%, or at least about 99%) in the second level of the substance or substances as compared to the first level of the substance or substances.

The terms “not substantially increased” “not substantially increased” refer to clinically insignificant changes (e.g., an increase) in the second level of a particular substance or particular substances (e.g., TCR clonotype) as compared to the first level of the particular substance or particular substances. In some embodiments, a not substantially increased second level of a particular substance or particular substances (e.g., TCR clonotype) is an increase in levels of less than about 10% (e.g., less than about 9%, less than about 8%, less than about 7%, less than about 6%, less than about 5%, less than about 4%, less than about 3%, less than about 2%, less than about 1%, less than about 0.5%, less than about 0.25%, less than about 0.2%, less than about 0.1%, less than about 0.05%, less than about 0.01%) as compared to the first level of TCR clonotype. In some embodiments, a not substantially increased level of a particular substance or particular substances (e.g., ctDNA) is a decrease of at least about 0.5% (e.g., at least about 1%, at least about 2%, at least about 4%, at least about 6%, at least about 8%, at least about 10%, at least about 12%, at least about 14%, at least about 16%, at least about 18%, at least about 20%, at least about 22%, at least about 24%, at least about 26%, at least about 28%, at least about 30%, at least about 40%, at least about 45%, at least about 50%, at least about 60%, at least about 65%, at least about 70%, at least about 75%, at least about 80%, at least about 85%, at least about 90%, at least about 95%, or at least about 99%) in the second level of the substance or substances as compared to the first level of the substance or substances.

A “chemotherapeutic agent” refers to a chemical compound useful in the treatment of a cancer. Chemotherapeutic agents include, e.g., “anti-hormonal agents” or “endocrine therapeutics” which act to regulate, reduce, block or inhibit the effects of hormones that can promote the growth of cancer. Additional classes, subclasses and examples of chemotherapeutic agents are known in the art.

The terms “acquired resistance” and “resistance” when used in reference to immunotherapy refer to a subsequent state of decreased effectiveness of the immunotherapy (e.g., when the immunotherapy was initially effective). As will be appreciated by those of ordinary skill in the art, resistance to immunotherapy can arise in a subject receiving immunotherapy treatment when a tumor cell in the subject develops a mutation or other molecular lesion that render the tumor cell resistant to the immunotherapy. In some embodiments, when a subject develops resistance to a first immunotherapy, a therapeutic intervention can be administered to the subject (e.g., the therapeutic intervention can be different from the first immunotherapy, including but not limited to, a different immunotherapy, a chemotherapy, a surgery, or any of the variety of other therapeutic interventions disclosed herein).

### Methods of Determining Efficacy of an Immunotherapy

In some embodiments, provided herein are methods of determining the efficacy of an immunotherapy in a subject, including: detecting a first level of circulating tumor DNA (ctDNA) in a biological sample isolated from the subject at a first time point; detecting a second level of ctDNA in a biological sample obtained from the subject at a second time point, wherein the subject has received at least one dose of an immunotherapy between the first time point and the second time point; and identifying the immunotherapy as being effective in a subject having a reduced second level of ctDNA as compared to the first level of ctDNA. In some embodiments, provided herein are methods of determining the efficacy of an immunotherapy in a subject, including: detecting a first level of at least one TCR clonotype in a biological sample isolated from the subject at a first time point; detecting a second level of the at least one TCR clonotype in a biological sample obtained from the subject at a second time point, wherein the subject has received at least one dose of an immunotherapy between the first time point and the second time point; and identifying the immunotherapy as being effective in a subject having an increased second level of the at least one TCR clonotype as compared to the first level of the at least one TCR clonotype. In some embodiments, provided herein are methods of determining the efficacy of an immunotherapy in a subject, including: detecting a first level of circulating tumor DNA (ctDNA) and a first level of at least one TCR clonotype in a biological sample isolated from the subject at a first time point; detecting a second level of ctDNA and a second level of the at least one TCR clonotype in a biological sample obtained from the subject at a second time point, wherein the subject has received at least one dose of an immunotherapy between the first time point and the second time point; and identifying the immunotherapy as being effective in a subject having: (i) a reduced second level of ctDNA as compared to the first level of ctDNA; and (ii) an increased second level of the at least one TCR clonotype as compared to the first level of the at least one TCR clonotype. In some embodiments, detecting and comparing both ctDNA levels and TCR clonotype levels at different time points is superior in determining the efficacy of an immunotherapy as compared to detecting and comparing either ctDNA levels or TCR clonotype levels individually. In some embodiments, detecting and comparing both ctDNA levels and TCR clonotype levels at different time points results in a more rapid determination of whether an immunotherapy is effective than conventional methods (e.g., imaging or scanning).

In some embodiments, an immunotherapy is determined to be effective when the amount of circulating tumor DNA (ctDNA) identified at the second time point is decreased by at least about 2-fold, at least about 3-fold, at least about 4-fold, at least about 5-fold, at least about 6-fold, at least about 7-fold, at least about 8-fold, at least about 9-fold, at least about 10-fold or more compared to the amount of circulating tumor DNA (ctDNA) identified at the first time point. In some embodiments, an immunotherapy is determined to be effective when the amount of circulating tumor DNA (ctDNA) identified at the second time point is decreased by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or more compared to the amount of circulating tumor DNA (ctDNA) identified at the first time point. In some embodiments, an immunotherapy is determined to be effective when circulating tumor DNA (ctDNA) is not observed at the second time point.

Additionally or alternatively, an immunotherapy is determined to be effective when the level of at least one TCR clonotype identified at the second time point is increased by at least about 1% (e.g., at least about 2%, at least about 4%, at least about 6%, at least about 8%, at least about 10%, at least about 12%, at least about 14%, at least about 16%, at least about 18%, at least about 20%, at least about 22%, at least about 24%, at least about 26%, at least about 28%, at least about 30%, at least about 40%, at least about 45%, at least about 50%, at least about 60%, at least about 65%, at least about 70%, at least about 75%, at least about 80%, at least about 85%, at least about 90%, at least about 95%, or at least about 99%) as compared to the first level of the at least one TCR clonotype.

In some embodiments, an immunotherapy is determined not to be effective (e.g., the immunotherapy has poor efficacy) when the amount of circulating tumor DNA (ctDNA) identified at the second time point is not substantially decreased (e.g., is less than about 10%, less than about 9%, less than about 8%, less than about 7%, less than about 6%, less than about 5%, less than about 4%, less than about 3%, less than about 2%, less than about 1%, less than about 0.5%, less than about 0.25%, less than about 0.2%, less than about 0.1%, less than about 0.05%, less than about 0.01%) as compared to the amount of circulating tumor DNA (ctDNA) identified at the first time point.

Additionally or alternatively, an immunotherapy is determined not to be effective (e.g., the immunotherapy has poor efficacy) when the second level of the at least one TCR clonotype is not substantially increased as compared to the first level of the at least one TCR clonotype (e.g., an increase in second level at least one TCR clonotype is less than about 10%, less than about 9%, less than about 8%, less than about 7%, less than about 6%, less than about 5%, less than about 4%, less than about 3%, less than about 2%, less than about 1%, less than about 0.5%, less than about 0.25%, less than about 0.2%, less than about 0.1%, less than about 0.05%, or less than about 0.01% as compared to the first level of the at least one TCR clonotype).

In some embodiments, methods provided herein for determining the efficacy of an immunotherapy include detecting the level of circulating tumor DNA (ctDNA) present in cell-free DNA, where the cell-free DNA is present in an amount less than about 1500 ng, e.g., less than about 1400 ng, less than about 1300 ng, less than about 1200 ng, less than about 1100 ng, less than about 1000 ng, less than about 900 ng, less than about 800 ng, less than about 700 ng, less than about 600 ng, less than about 500 ng, less than about 400 ng, less than about 300 ng, less than about 200 ng, less than about 150 ng, less than about 100 ng, less than about 95 ng, less than about 90 ng, less than about 85 ng, less than about 80 ng, less than about 75 ng, less than about 70 ng, less than about 65 ng, less than about 60 ng, less than about 55 ng, less than about 50 ng, less than about 45 ng, less than about 40 ng, less than about 35 ng, less than about 30 ng, less than about 25 ng, less than about 20 ng, less than about 15 ng, less than about 10 ng, or less than about 5 ng.

In some embodiments, methods provided herein for determining the efficacy of an immunotherapy include detecting the circulating tumor DNA (ctDNA) present in cell-free DNA, where the circulating tumor DNA represents 100% of the cell-free DNA. In some embodiments, methods provided herein for determining the efficacy of an immunotherapy include detecting the level of circulating tumor DNA (ctDNA) present in cell-free DNA, where the circulating tumor DNA represents less than 100% of the cell-free DNA, e.g. about 95%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5%, about 4%, about 3%, about 2%, about 1%, about 0.95%, about 0.90%, about 0.85%, about 0.80%, about 0.75%, about 0.70%, about 0.65%, about 0.60%, about 0.55%, about 0.50%, about 0.45%, about 0.40%, about 0.35%, about 0.30%, about 0.25%, about 0.20%, about 0.15%, about 0.10%, about 0.09%, about 0.08%, about 0.07%, about 0.06%, about 0.05% of the cell-free DNA, or less.

In some embodiments, after determining the efficacy of an immunotherapy administered to a subject, the subject can be administered a diagnostic test (e.g., any of the diagnostic tests disclosed herein) and/or monitored (e.g., according to any of the monitoring methods, schedules, etc. disclosed herein). In some embodiments, after determining the efficacy of an immunotherapy administered to a subject, the subject can be selected for further diagnostic testing (e.g., using any of the diagnostic tests disclosed herein) and/or selected for increased monitoring (e.g., according to any of the increased monitoring methods, schedules, etc. disclosed herein). For example, a subject can be administered an immunotherapy, which immunotherapy is determined to be effective, and the subject can then be administered a diagnostic test and/or selected for further diagnostic testing (e.g., to confirm the effectiveness of the immunotherapy). As another example, a subject can be administered an immunotherapy, which immunotherapy is determined to be effective, and the subject can then be monitored and/or selected for increased monitoring (e.g., to keep watch for the reemergence of the same or another cancer).

In some embodiments, an immunotherapy is determined to be effective in a subject. In such embodiments, the subject may be administered one or more additional doses of the effective immunotherapy during the course of treatment. In some embodiments, when an immunotherapy is determined to be effective in a subject, the subject may be administered one or more additional doses of the effective immunotherapy during the course of treatment without being administered other therapeutic interventions (e.g. other therapeutic interventions to treat the same condition the immunotherapy treats, e.g., cancer). In some embodiments, when an immunotherapy is determined to be effective in a subject, the subject may be administered one or more additional doses of the effective immunotherapy, and may further be administered one or more therapeutic interventions (e.g., any of the therapeutic interventions disclosed herein) during the course of treatment.

In some embodiments, an immunotherapy is determined not to be effective in a subject (e.g., the immunotherapy has poor efficacy). In such embodiments, the subject may be administered a therapeutic intervention (e.g., any of the therapeutic interventions disclosed herein) that is different that the ineffective immunotherapy during the course of treatment. As non-limiting examples, a subject may be administered a different immunotherapy, a targeted therapy, a chemotherapy, radiation therapy, and/or surgery. Those of ordinary skill in the art will be aware of suitable therapeutic interventions to administer when the immunotherapy is determined not to be effective.

In some aspects, methods provided herein include obtaining from the subject additional sample(s) at additional time point(s) (e.g., at a third time point, a fourth time point, etc.) and determining the efficacy of an immunotherapy at the additional time point(s).

In some aspects, the second time point is about one to about ten weeks (e.g., about one to about nine weeks, about one to about eight weeks, about one to about seven weeks, about one to about six weeks, about one to about five weeks, about one to about four weeks, about one to about three weeks, about one to about two weeks, about two to about ten weeks, about two to about nine weeks, about two to about eight weeks, about two to about seven weeks, about two to about six weeks, about two to about five weeks, about two to about four weeks, about two to about three weeks, about three to about ten weeks, about three to about nine weeks, about three to about eight weeks, about three to about seven weeks, about three to about six weeks, about three to about five weeks, about three to about four weeks, about four to about ten weeks, about four to about nine weeks, about four to about eight weeks, about four to about seven weeks, about four to about six weeks, about four to about five weeks, about five to about ten weeks, about five to about nine weeks, about five to about eight weeks, about five to about seven weeks, about five to about six weeks, about six to about ten weeks, about six to about nine weeks, about six to about eight weeks, about six to about seven weeks, about seven to about ten weeks, about seven to about nine weeks, about seven to about eight weeks, about eight to about ten weeks, about eight to about nine weeks, about nine to about ten weeks; or about one week, about two weeks, about three weeks, about four weeks, about five weeks, about six weeks, about seven weeks, about eight weeks, about nine weeks, about ten weeks) after the first time point.

### Determining, Monitoring, and Treating Resistance to an Immunotherapy

Also provided herein are methods for determining that a subject that has developed resistance to an immunotherapy (e.g., any of the immunotherapies disclosed herein or known in the art), methods for monitoring a subject for the development of resistance to an immunotherapy, and methods for treating such subjects with a different therapeutic intervention.

In some embodiments, provided herein are methods of determining that a subject has not developed resistance to an immunotherapy (e.g., any of the immunotherapies disclosed herein or known in the art), including: detecting a first level of circulating tumor DNA (ctDNA) in a biological sample isolated from the subject at a first time point; detecting a second level of ctDNA in a biological sample obtained from the subject at a second time point, wherein the subject has received at least one dose of an immunotherapy between the first time point and the second time point; and identifying the subject as not having developed resistance to an immunotherapy when the second level of ctDNA is reduced as compared to the first level of ctDNA. In some embodiments, provided herein are methods of determining that a subject has developed resistance to an immunotherapy (e.g., any of the immunotherapies disclosed herein or known in the art), including: detecting a first level of ctDNA in a biological sample isolated from the subject at a first time point; detecting a second level of ctDNA in a biological sample obtained from the subject at a second time point, wherein the subject has received at least one dose of an immunotherapy between the first time point and the second time point; and identifying the subject as having developed resistance when the second level of ctDNA is not substantially reduced as compared to the first level of ctDNA. In some embodiments, the subject determined to have developed resistance to the immunotherapy exhibits a decreased level of ctDNA at a time point between the first and second time points (e.g., the level of ctDNA initially decreases upon administration of the immunotherapy, but then increases when the subject develops resistance).

In some embodiments, a subject is determined not to have developed resistance to an immunotherapy when the amount of circulating tumor DNA (ctDNA) identified at the second time point is decreased by at least about 2-fold, at least about 3-fold, at least about 4-fold, at least about 5-fold, at least about 6-fold, at least about 7-fold, at least about 8-fold, at least about 9-fold, at least about 10-fold or more compared to the amount of circulating tumor DNA (ctDNA) identified at the first time point. In some embodiments, a subject is determined not to have developed resistance to an immunotherapy when the amount of circulating tumor DNA (ctDNA) identified at the second time point is decreased by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or more compared to the amount of circulating tumor DNA (ctDNA) identified at the first time point. In some embodiments, a subject is determined not to have developed resistance to an immunotherapy when circulating tumor DNA (ctDNA) is not observed at the second time point

In some embodiments, provided herein are methods of determining that a subject has not developed resistance to an immunotherapy (e.g., any of the immunotherapies disclosed herein or known in the art), including: detecting a first level of at least one TCR clonotype in a biological sample isolated from the subject at a first time point; detecting a second level of the at least one TCR clonotype in a biological sample obtained from the subject at a second time point, wherein the subject has received at least one dose of an immunotherapy between the first time point and the second time point; and identifying the subject as not having developed resistance to an immunotherapy when the second level of the at least one TCR clonotype is increased as compared to the first level of the at least one TCR clonotype. In some embodiments, provided herein are methods of determining that a subject has developed resistance to an immunotherapy (e.g., any of the immunotherapies disclosed herein or known in the art), including: detecting a first level of at least one TCR clonotype in a biological sample isolated from the subject at a first time point; detecting a second level of the at least one TCR clonotype in a biological sample obtained from the subject at a second time point, wherein the subject has received at least one dose of an immunotherapy between the first time point and the second time point; and identifying the subject as having developed resistance when the second level of the at least one TCR clonotype is not substantially increased as compared to the first level of the at least one TCR clonotype. In some embodiments, the subject determined to have developed resistance to the immunotherapy exhibits an increased level of the at least one TCR clonotype at a time point between the first and second time points (e.g., the level of the at least one TCR clonotype initially increases upon administration of the immunotherapy, but then decreases when the subject develops resistance).

In some embodiments, a subject is identified as having developed resistance to an administered immunotherapy when the second level of the at least one TCR clonotype is not substantially increased as compared to the first level of the at least one TCR clonotype (e.g., an increase in second level at least one TCR clonotype of less than about 10%, less than about 9%, less than about 8%, less than about 7%, less than about 6%, less than about 5%, less than about 4%, less than about 3%, less than about 2%, less than about 1%, less than about 0.5%, less than about 0.25%, less than about 0.2%, less than about 0.1%, less than about 0.05%, or less than about 0.01% as compared to the first level of the at least one TCR clonotype.

In some embodiments, detecting and comparing both ctDNA levels and TCR clonotype levels at different time points is superior in determining whether or not a subject has developed resistance to an immunotherapy as compared to detecting and comparing either ctDNA levels or TCR clonotype levels individually. In some embodiments, detecting and comparing both ctDNA levels and TCR clonotype levels at different time points results in a more rapid determination of whether the subject has developed resistance than conventional methods (e.g., imaging or scanning).

In some embodiments, methods of determining that a subject that has developed resistance to an immunotherapy (e.g., any of the immunotherapies disclosed herein or known in the art) include using any of the methods disclosed herein for detecting the presence or level of circulating tumor DNA (ctDNA). In some embodiments, a subject is determined to have developed resistance to an immunotherapy when that immunotherapy is no longer effective or is less effective than it was when first administered. For example, a subject can be determined to have developed resistance to an immunotherapy when the immunotherapy is at least 20%, 25%, 30%, 35%, 40% 45%, 50%, 55%, 60%, 65%, 70%, 75%, 80%, 85%, 90%, 95%, or 100%, or any percentage within between, less effective than when the immunotherapy was first administered. The effectiveness of an immunotherapy, both when it is first administered and during the course of the treatment, can be determined by any of a variety of methods and techniques. For example, the size and/or position of the tumor (as determined, e.g., by scanning or imaging technologies), the number of cancer cells, the amount of cell-free DNA, and/or the amount of circulating tumor DNA can be determined and used to assess whether a subject has developed resistance to the immunotherapy. Other suitable methods and techniques are known in the art. In some embodiments, after determining that a subject that has developed resistance to an immunotherapy, a different immunotherapy and/or therapeutic intervention (e.g., any of the therapeutic interventions disclosed herein or known in the art) is selected and/or administered to the subject.

In some embodiments, methods for monitoring a subject for the development of resistance to an immunotherapy (e.g., any of the immunotherapies disclosed herein or known in the art) include using any of the methods disclosed herein for detecting the presence or level of circulating tumor DNA (ctDNA).

In some embodiments, methods for treating a subject that has developed resistance to a therapeutic intervention (e.g., any of the therapeutic interventions disclosed herein or known in the art) include using any of the methods disclosed herein for detecting circulating tumor DNA.

In some embodiments, methods provided herein for determining that a subject that has developed resistance to an immunotherapy, for monitoring a subject for the development of resistance to an immunotherapy, and/or for treating such subjects with a different therapeutic intervention include determining the presence or level of circulating tumor DNA present in cell-free DNA, where the cell-free DNA is present in an amount less than about 1500 ng, e.g., less than about 1400 ng, less than about 1300 ng, less than about 1200 ng, less than about 1100 ng, less than about 1000 ng, less than about 900 ng, less than about 800 ng, less than about 700 ng, less than about 600 ng, less than about 500 ng, less than about 400 ng, less than about 300 ng, less than about 200 ng, less than about 150 ng, less than about 100 ng, less than about 95 ng, less than about 90 ng, less than about 85 ng, less than about 80 ng, less than about 75 ng, less than about 70 ng, less than about 65 ng, less than about 60 ng, less than about 55 ng, less than about 50 ng, less than about 45 ng, less than about 40 ng, less than about 35 ng, less than about 30 ng, less than about 25 ng, less than about 20 ng, less than about 15 ng, less than about 10 ng, or less than about 5 ng.

In some embodiments methods provided herein for determining that a subject that has developed resistance to an immunotherapy, for monitoring a subject for the development of resistance to an immunotherapy, and/or for treating such subjects with a different therapeutic intervention include determining the level of circulating tumor DNA present in cell-free DNA, where the circulating tumor DNA represents 100% of the cell-free DNA. In some embodiments, methods provided herein for determining that a subject that has developed resistance to an immunotherapy, for monitoring a subject for the development of resistance to an immunotherapy, and/or for treating such subjects with a different therapeutic intervention include determining the level of circulating tumor DNA present in cell-free DNA, where the circulating tumor DNA represents less than 100% of the cell-free DNA, e.g. about 95%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5%, about 4%, about 3%, about 2%, about 1%, about 0.95%, about 0.90%, about 0.85%, about 0.80%, about 0.75%, about 0.70%, about 0.65%, about 0.60%, about 0.55%, about 0.50%, about 0.45%, about 0.40%, about 0.35%, about 0.30%, about 0.25%, about 0.20%, about 0.15%, about 0.10%, about 0.09%, about 0.08%, about 0.07%, about 0.06%, about 0.05% of the cell-free DNA, or less.

In some aspects, methods provided herein include obtaining from the subject additional sample(s) at additional time point(s) (e.g., at a third time point, a fourth time point, etc.) and determining whether a subject has developed resistance to an immunotherapy at the additional time point(s).

In some embodiments, after determining that a subject has developed resistance to the administered immunotherapy, the subject can be administered a diagnostic test (e.g., any of the diagnostic tests disclosed herein) and/or monitored (e.g., according to any of the monitoring methods, schedules, etc. disclosed herein). In some embodiments, after determining that a subject has developed resistance to the administered immunotherapy, the subject can be selected for further diagnostic testing (e.g., using any of the diagnostic tests disclosed herein) and/or selected for increased monitoring (e.g., according to any of the increased monitoring methods, schedules, etc. disclosed herein). For example, a subject can be administered an immunotherapy, for which the subject has developed resistance to, and the subject can then be administered a diagnostic test and/or selected for further diagnostic testing (e.g., to confirm the effectiveness of the immunotherapy). As another example, a subject can be administered an immunotherapy for which the subject has not been identified as having developed resistance, and the subject can then be monitored and/or selected for increased monitoring (e.g., to keep watch for the reemergence of the same or another cancer).

In some embodiments where the subject has been identified as not having developed resistance to an immunotherapy, the subject may be administered one or more additional doses of the effective immunotherapy during the course of treatment (i.e. the immunotherapy for which the subject has not developed resistance). In some embodiments, the subject may be administered one or more additional doses of the immunotherapy during the course of treatment without being administered other therapeutic interventions (e.g. other therapeutic interventions to treat the same condition the immunotherapy treats, e.g., cancer). In some embodiments, the subject may be administered one or more additional doses of the effective immunotherapy, and may further be administered one or more therapeutic interventions (e.g., any of the therapeutic interventions disclosed herein) during the course of treatment.

In some embodiments where a subject is identified as having developed resistance to an immunotherapy, the subject may be administered a therapeutic intervention (e.g., any of the therapeutic interventions disclosed herein) that is different than the immunotherapy for which the subject has developed resistance during the course of treatment. As non-limiting examples, a subject may be administered a different immunotherapy, a targeted therapy, a chemotherapy, radiation therapy, and/or surgery. Those of ordinary skill in the art will be aware of suitable therapeutic interventions to administer when the subject has developed resistance.

In some aspects, methods provided herein include obtaining from the subject additional sample(s) at additional time point(s) (e.g., at a third time point, a fourth time point, etc.) and determining the efficacy of an immunotherapy at the additional time point(s).

In some aspects, the second time point is about one to about ten weeks (e.g., about one to about nine weeks, about one to about eight weeks, about one to about seven weeks, about one to about six weeks, about one to about five weeks, about one to about four weeks, about one to about three weeks, about one to about two weeks, about two to about ten weeks, about two to about nine weeks, about two to about eight weeks, about two to about seven weeks, about two to about six weeks, about two to about five weeks, about two to about four weeks, about two to about three weeks, about three to about ten weeks, about three to about nine weeks, about three to about eight weeks, about three to about seven weeks, about three to about six weeks, about three to about five weeks, about three to about four weeks, about four to about ten weeks, about four to about nine weeks, about four to about eight weeks, about four to about seven weeks, about four to about six weeks, about four to about five weeks, about five to about ten weeks, about five to about nine weeks, about five to about eight weeks, about five to about seven weeks, about five to about six weeks, about six to about ten weeks, about six to about nine weeks, about six to about eight weeks, about six to about seven weeks, about seven to about ten weeks, about seven to about nine weeks, about seven to about eight weeks, about eight to about ten weeks, about eight to about nine weeks, about nine to about ten weeks; or about one week, about two weeks, about three weeks, about four weeks, about five weeks, about six weeks, about seven weeks, about eight weeks, about nine weeks, about ten weeks) after the first time point.

Determining Cell-Free Tumor Load (ctFL) in a Subject

Also provided herein are methods for determining cell-free tumor load (cfTL). In some embodiments, cfTL is detected in a biological sample isolated from the subject at a first time point. In some embodiments, cfTL is detected in a biological sample isolated from the subject at a second time point. In some embodiments, the subject has received at least one dose of the targeted therapy between the first time point and the second time point.

In some embodiments, determining cell-free tumor load (cfTL) in a subject includes detecting a first level of at least one genetic alteration present in ctDNA and/or a first level of aneuploidy in a biological sample isolated from the subject at a first time point. In some embodiments, determining cell-free tumor load (cfTL) in a subject includes detecting a first level of at least one genetic alteration present in ctDNA and/or a first level of aneuploidy in a biological sample isolated from the subject at a second time point. In some embodiments, the subject has received at least one dose of the targeted therapy between the first time point and the second time point.

In some embodiments, determining cell-free tumor load (cfTL) in a subject includes detecting the level of at least one genetic alteration (e.g., at least 2, 3, 4, 5, 6, 7, 8, 9, 10, or more genetic alterations) present in ctDNA present in a biological sample isolated from the subject. In some embodiments, no genetic alterations are detected in the subject, and determining cell-free tumor load (cfTL) in a subject includes detecting the level of aneuploidy in the subject.

In some embodiments, the subject exhibits a cfTL that is reduced at a second time point as compared to a first time point. In some embodiments, the subject exhibits a cfTL that is not reduced at a second time point as compared to a first time point.

In some embodiments, the efficacy of an immunotherapy can be determined by determining cfTL in a subject (e.g., by detecting levels of at least one genetic alteration present in ctDNA and/or levels of aneuploidy) in combination with detecting the level at least one TCR clonotype in the subject (e.g., by any of the variety of methods disclosed herein). In some embodiments, detecting and comparing both cfTL and TCR clonotype levels at different time points is superior in determining the efficacy of an immunotherapy as compared to detecting and comparing either cfTL or TCR clonotype levels individually. In some embodiments, detecting and comparing both cfTL and TCR clonotype levels at different time points results in a more rapid determination of whether an immunotherapy is effective than conventional methods (e.g., imaging or scanning).

In some embodiments, the second cfTL is not substantially reduced as compared to the first cfTL (e.g., an increase in second cfTL of less than about 10%, less than about 9%, less than about 8%, less than about 7%, less than about 6%, less than about 5%, less than about 4%, less than about 3%, less than about 2%, less than about 1%, less than about 0.5%, less than about 0.25%, less than about 0.2%, less than about 0.1%, less than about 0.05%, or less than about 0.01% as compared to the first cfTL, or a decrease of at least about 0.5%, at least about 1%, at least about 2%, at least about 4%, at least about 6%, at least about 8%, at least about 10%, at least about 12%, at least about 14%, at least about 16%, at least about 18%, at least about 20%, at least about 22%, at least about 24%, at least about 26%, at least about 28%, at least about 30%, at least about 40%, at least about 45%, at least about 50%, at least about 60%, at least about 65%, at least about 70%, at least about 75%, at least about 80%, at least about 85%, at least about 90%, at least about 95%, or at least about 99% in the second cfTL as compared to the first cfTL.

In some aspects, methods provided herein include obtaining from the subject additional sample(s) at additional time point(s) (e.g., at a third time point, a fourth time point, etc.) and determining whether a subject has developed resistance to a targeted therapy at the additional time point(s).

In some aspects, the second time point is about one to about four weeks (e.g., about one to about three weeks, about one to about two weeks, about two to about four weeks, about two to about three weeks, about three weeks to about four weeks; about 2 days to about 30 days, about 2 days to about 28 days, about 2 days to about 26 days, about 2 days to about 24 days, about 2 days to about 22 days, about 2 days to about 20 days, about 2 days to about 18 days, about 2 days to about 16 days, about 2 days to about 14 days, about 2 days to about 12 days, about 2 days to about 10 days, about 2 days to about 8 days, about 2 days to about 6 days, about 2 days to about 4 days, about 4 days to about 30, about 4 days to about 28 days, about 4 days to about 26 days, about 4 days to about 24 days, about 4 days to about 22 days, about 4 days to about 20 days, about 4 days to about 18 days, about 4 days to about 16 days, about 4 days to about 14 days, about 4 days to about 12 days, about 4 days to about 10 days, about 4 days to about 8 days, about 4 days to about 6 days, about 6 days to about 30, about 6 days to about 28 days, about 6 days to about 26 days, about 6 days to about 24 days, about 6 days to about 22 days, about 6 days to about 20 days, about 6 days to about 18 days, about 6 days to about 16 days, about 6 days to about 14 days, about 6 days to about 12 days, about 6 days to about 10 days, about 6 days to about 8 days, about 8 days to about 30, about 8 days to about 28 days, about 8 days to about 26 days, about 8 days to about 24 days, about 8 days to about 22 days, about 8 days to about 20 days, about 8 days to about 18 days, about 8 days to about 16 days, about 8 days to about 14 days, about 8 days to about 12 days, about 8 days to about 10 days, about 10 days to about 30, about 10 days to about 28 days, about 10 days to about 26 days, about 10 days to about 24 days, about 10 days to about 22 days, about 10 days to about 20 days, about 10 days to about 18 days, about 10 days to about 16 days, about 10 days to about 14 days, about 10 days to about 12 days, about 12 days to about 30, about 12 days to about 28 days, about 12 days to about 26 days, about 12 days to about 24 days, about 12 days to about 22 days, about 12 days to about 20 days, about 12 days to about 18 days, about 12 days to about 16 days, about 12 days to about 14 days, about 14 days to about 30, about 14 days to about 28 days, about 14 days to about 26 days, about 14 days to about 24 days, about 14 days to about 22 days, about 14 days to about 20 days, about 14 days to about 18 days, about 14 days to about 16 days, about 16 days to about 30, about 16 days to about 28 days, about 16 days to about 26 days, about 16 days to about 24 days, about 16 days to about 22 days, about 16 days to about 20 days, about 16 days to about 18 days, about 18 days to about 30, about 18 days to about 28 days, about 18 days to about 26 days, about 18 days to about 24 days, about 18 days to about 22 days, about 18 days to about 20 days, about 20 days to about 30, about 20 days to about 28 days, about 20 days to about 26 days, about 20 days to about 24 days, about 20 days to about 22 days, about 22 days to about 30, about 22 days to about 28 days, about 22 days to about 26 days, about 22 days to about 24 days, about 24 days to about 30, about 24 days to about 28 days, about 24 days to about 26 days, about 26 days to about 30, about 26 days to about 28 days, about 26 days to about 30; about 1 day, about 2 days, about 4 days, about 6 days, about 8 days, about 10 days, about 12 days, about 14 days, about 16 days, about 18 days, about 20 days, about 22 days, about 24 days, about 26 days, about 28 days, or about 30 days) after the first time point.

In some aspects, the second time point is about 1 hour to about 7 days (e.g., about 1 hour to about 6 days, about 1 hour to about 5 days, about 1 hour to about 4 days, about 1 hour to about 72 hours, about 1 hour to about 66 hours, about 1 hour to about 60 hours, about 1 hour to about 54 hours, about 1 hour to about 48 hours, about 1 hour to about 42 hours, about 1 hour to about 36 hours, about 1 hour to about 30 hours, about 1 hour to about 24 hours, about 1 hour to about 18 hours, about 1 hour to about 12 hours, about 1 hour to about 6 hours, about 1 hour to about 4 hours, about 1 hour to about 2 hours, about 2 hours to about 7 days, about 2 hours to about 6 days, about 2 hours to about 5 days, about 2 hours to about 4 days, about 2 hours to about 72 hours, about 2 hours to about 66 hours, about 2 hours to about 60 hours, about 2 hours to about 54 hours, about 2 hours to about 48 hours, about 2 hours to about 42 hours, about 2 hours to about 36 hours, about 2 hours to about 30 hours, about 2 hours to about 24 hours, about 2 hours to about 18 hours, about 2 hours to about 12 hours, about 2 hours to about 6 hours, about 2 hours to about 4 hours, about 4 hours to about 7 days, about 4 hours to about 6 days, about 4 hours to about 5 days, about 4 hours to about 4 days, about 4 hours to about 72 hours, about 4 hours to about 66 hours, about 4 hours to about 60 hours, about 4 hours to about 54 hours, about 4 hours to about 48 hours, about 4 hours to about 42 hours, about 4 hours to about 36 hours, about 4 hours to about 30 hours, about 4 hours to about 24 hours, about 4 hours to about 18 hours, about 4 hours to about 12 hours, about 4 hours to about 6 hours, about 6 hours to about 7 days, about 6 hours to about 6 days, about 6 hours to about 5 days, about 6 hours to about 4 days, about 6 hours to about 72 hours, about 6 hours to about 66 hours, about 6 hours to about 60 hours, about 6 hours to about 54 hours, about 6 hours to about 48 hours, about 6 hours to about 42 hours, about 6 hours to about 36 hours, about 6 hours to about 30 hours, about 6 hours to about 24 hours, about 6 hours to about 18 hours, about 6 hours to about 12 hours, about 12 hours to about 7 days, about 12 hours to about 6 days, about 12 hours to about 5 days, about 12 hours to about 4 days, about 12 hours to about 72 hours, about 12 hours to about 66 hours, about 12 hours to about 60 hours, about 12 hours to about 54 hours, about 12 hours to about 48 hours, about 12 hours to about 42 hours, about 12 hours to about 36 hours, about 12 hours to about 30 hours, about 12 hours to about 24 hours, about 12 hours to about 18 hours, about 18 hours to about 7 days, about 18 hours to about 6 days, about 18 hours to about 5 days, about 18 hours to about 4 days, about 18 hours to about 72 hours, about 18 hours to about 66 hours, about 18 hours to about 60 hours, about 18 hours to about 54 hours, about 18 hours to about 48 hours, about 18 hours to about 42 hours, about 18 hours to about 36 hours, about 18 hours to about 30 hours, about 18 hours to about 24 hours, about 24 hours to about 7 days, about 24 hours to about 6 days, about 24 hours to about 5 days, about 24 hours to about 4 days, about 24 hours to about 72 hours, about 24 hours to about 66 hours, about 24 hours to about 60 hours, about 24 hours to about 54 hours, about 24 hours to about 48 hours, about 24 hours to about 42 hours, about 24 hours to about 36 hours, about 24 hours to about 30 hours, about 30 hours to about 7 days, about 30 hours to about 6 days, about 30 hours to about 5 days, about 30 hours to about 4 days, about 30 hours to about 72 hours, about 30 hours to about 66 hours, about 30 hours to about 60 hours, about 30 hours to about 54 hours, about 30 hours to about 48 hours, about 30 hours to about 42 hours, about 30 hours to about 36 hours, about 36 hours to about 7 days, about 36 hours to about 6 days, about 36 hours to about 5 days, about 36 hours to about 4 days, about 36 hours to about 72 hours, about 36 hours to about 66 hours, about 36 hours to about 60 hours, about 36 hours to about 54 hours, about 36 hours to about 48 hours, about 36 hours to about 42 hours, about 42 hours to about 7 days, about 42 hours to about 6 days, about 42 hours to about 5 days, about 42 hours to about 4 days, about 42 hours to about 72 hours, about 42 hours to about 66 hours, about 42 hours to about 60 hours, about 42 hours to about 54 hours, about 42 hours to about 48 hours, about 48 hours to about 7 days, about 48 hours to about 6 days, about 48 hours to about 5 days, about 48 hours to about 4 days, about 48 hours to about 72 hours, about 48 hours to about 66 hours, about 48 hours to about 60 hours, about 48 hours to about 54 hours, about 54 hours to about 7 days, about 54 hours to about 6 days, about 54 hours to about 5 days, about 54 hours to about 4 days, about 54 hours to about 72 hours, about 54 hours to about 66 hours, about 54 hours to about 60 hours, about 60 hours to about 7 days, about 60 hours to about 6 days, about 60 hours to about 5 days, about 60 hours to about 4 days, about 60 hours to about 72 hours, about 60 hours to about 66 hours, about 66 hours to about 7 days, about 66 hours to about 6 days, about 66 hours to about 5 days, about 66 hours to about 4 days, about 66 hours to about 72 hours, about 72 hours to about 7 days, about 72 hours to about 6 days, about 72 hours to about 5 days, about 72 hours to about 4 days, about 4 days to about 7 days, about 4 days to about 6 days, about 4 days to about 5 days, about 5 days to about 7 days, about 5 days to about 6 days, about 6 days to about 7 days; about 1 hour, about 2 hours, about 4 hours, about 6 hours, about 8 hours, about 10 hours, about 12 hours, about 18 hours, about 24 hours, about 30 hours, about 36 hours, about 42 hours, about 48 hours, about 54 hours, about 60 hours, about 66 hours, about 72 hours, about 4 days, about 5 days, about 6 days, or about 7 days) after the first time point.

### Identifying the Presence or Levels of Circulating Tumor DNA in a Subject

Provided herein are methods for identifying the presence or level of circulating tumor DNA (ctDNA) in a subject (e.g., a first level of ctDNA at a first time point and/or a second level of ctDNA at a second time point). In some embodiments, methods for identifying the presence or level of circulating tumor DNA in a subject include detecting one or more genetic alterations in cell-free DNA in a biological sample isolated from the subject (e.g., using a method designed to detect genetic alterations such as, without limitation, TEC-Seq). In some embodiments, the presence of circulating tumor DNA indicates the presence of a cancer cell in the subject (e.g., a cancer cell from any of the exemplary cancers described herein). In some embodiments, the level of circulating tumor DNA indicates the tumor burden in the subject.

In some embodiments, the biological sample is isolated from subject. Any suitable biological sample that contains cell-free DNA (e.g., cell-free DNA that includes ctDNA) can be used in accordance with any of the variety of methods disclosed herein. For example, the biological sample can include blood, plasma, serum, urine, cerebrospinal fluid, saliva, sputum, broncho-alveolar lavage, bile, lymphatic fluid, cyst fluid, stool, uterine lavage, vaginal fluids, ascites, and combinations thereof. Methods of isolating biological samples from a subject are known to those of ordinary skill in the art.

In some embodiments, detecting the presence or level of ctDNA is performed using one or more of the methods described herein (e.g., a targeted capture method, a next-generation sequencing method, and an array-based method, or any combinations thereof). In some embodiments, detecting the presence or level of ctDNA is performed using TEC-Seq, or a variation of TEC-Seq (Phallen et al., Science Transl Med, (403), 2017). For example, detecting the presence or level of ctDNA can include the following steps: extracting cell-free DNA from blood, ligating a low complexity pool of dual index barcode adapters to the cell-free DNA to generate a plurality of barcode adapter-ligated cell-free DNA segments, capturing the plurality of barcode adapter-ligated cell-free DNA segments, sequencing the plurality of captured barcode adapter-ligated cell-free DNA segments, aligning the sequenced plurality of captured barcode adapter-ligated cell-free DNA segments to a reference genome, and identifying sequence alterations using aligned sequences of multiple distinct molecules containing identical redundant changes. In some embodiments, the presence or level of ctDNA is detected (e.g., using a TEC-Seq approach) at two or more time points (e.g., a first time point prior to administration of an immunotherapy and a second time point after administration of the immunotherapy). In some embodiments, an increase in the number or level of sequence alterations (e.g., at a second time point) indicates an increase in the level of ctDNA. In some embodiments, an increase in the level of ctDNA indicates an increased tumor load or tumor burden in the subject. In some embodiments, a decrease in the number or level of sequence alterations (e.g., at a second time point) indicates a decrease in the level of ctDNA. In some embodiments, a decrease in the level of ctDNA indicates a decreased tumor load or tumor burden in the subject.

In some embodiments, detecting the presence or level of ctDNA is performed using sequencing technology (e.g., a next-generation). A variety of sequencing technologies are known in the art. For example, a variety of technologies for detection and characterization of circulating tumor DNA in cell-free DNA is described in Haber and Velculescu, Blood-Based Analyses of Cancer: Circulating Tumor Cells and Circulating Tumor DNA, Cancer Discov., June; 4(6):650-61. doi: 10.1158/2159-8290.CD-13-1014, 2014, incorporated herein by reference in its entirety. Non-limiting examples of such techniques include SafeSeqs (Kinde et. al, Detection and quantification of rare mutations with massively parallel sequencing, Proc Natl Acad Sci USA; 108, 9530-5, 2011), OnTarget (Forshew et al., Noninvasive identification and monitoring of cancer mutations by targeted deep sequencing of plasma DNA, Sci Transl Med; 4:136ra68, 2012), and TamSeq (Thompson et al., Winnowing DNA for rare sequences: highly specific sequence and methylation based enrichment. PLoS ONE, 7:e31597, 2012), each of which is incorporated herein by reference in its entirety. In some embodiments, detecting the presence or level of ctDNA is performed using droplet digital PCR (ddPCR. In some embodiments, detecting the presence or level of ctDNA is performed using other sequencing technologies, including but not limited to, chain-termination techniques, shotgun techniques, sequencing-by-synthesis methods, methods that utilize microfluidics, other capture technologies, or any of the other sequencing techniques known in the art that are useful for detection of small amounts of DNA in a sample (e.g., circulating tumor DNA in a cell-free DNA sample).

In some embodiments, detecting the presence or level of ctDNA is performed using array-based methods. For example, detecting the presence or level of ctDNA can be performed using a DNA microarray. In some embodiments, a DNA microarray can detect the presence or level of ctDNA. In some embodiments, cell-free DNA is amplified prior to detecting the presence or level of ctDNA. Non-limiting examples of array-based methods that can be used in any of the methods described herein, include: a complementary DNA (cDNA) microarray (Kumar et al. (2012) J. Pharm. Bioallied Sci. 4(1): 21-26; Laere et al. (2009) Methods Mol. Biol. 512: 71-98; Mackay et al. (2003) Oncogene 22: 2680-2688; Alizadeh et al. (1996) Nat. Genet. 14: 457-460), an oligonucleotide microarray (Kim et al. (2006) Carcinogenesis 27(3): 392-404; Lodes et al. (2009) PLoS One 4(7): e6229), a bacterial artificial chromosome (BAC) clone chip (Chung et al. (2004) Genome Res. 14(1): 188-196; Thomas et al. (2005) Genome Res. 15(12): 1831-1837), a single-nucleotide polymorphism (SNP) microarray (Mao et al. (2007) Curr. Genomics 8(4): 219-228; Jasmine et al. (2012) PLoS One 7(2): e31968), a microarray-based comparative genomic hybridization array (array-CGH) (Beers and Nederlof (2006) Breast Cancer Res. 8(3): 210; Pinkel et al. (2005) Nat. Genetics 37: S11-S17; Michels et al. (2007) Genet. Med. 9: 574-584), a molecular inversion probe (MIP) assay (Wang et al. (2012) Cancer Genet 205(7-8): 341-55; Lin et al. (2010) BMC Genomics 11: 712). In some embodiments, the cDNA microarray is an Affymetrix microarray (Irizarry (2003) Nucleic Acids Res 31:e15; Dalma-Weiszhausz et al. (2006) Methods Enzymol. 410: 3-28), a NimbleGen microarray (Wei et al. (2008) Nucleic Acids Res 36(9): 2926-2938; Albert et al. (2007) Nat. Methods 4: 903-905), an Agilent microarray (Hughes et al. (2001) Nat. Biotechnol. 19(4): 342-347), or a BeadArray array (Liu et al. (2017) Biosens Bioelectron 92: 596-601). In some embodiments, the oligonucleotide microarray is a DNA tiling array (Mockler and Ecker (2005) Genomics 85(1): 1-15; Bertone et al. (2006) Genome Res 16(2): 271-281). Other suitable array-based methods are known in the art.

In some embodiments, methods provided herein can be used to detect the presence or level of ctDNA in cell-free DNA, where the cell-free DNA is present in an amount less than about 1500 ng, e.g., less than about 1400 ng, less than about 1300 ng, less than about 1200 ng, less than about 1100 ng, less than about 1000 ng, less than about 900 ng, less than about 800 ng, less than about 700 ng, less than about 600 ng, less than about 500 ng, less than about 400 ng, less than about 300 ng, less than about 200 ng, less than about 150 ng, less than about 100 ng, less than about 95 ng, less than about 90 ng, less than about 85 ng, less than about 80 ng, less than about 75 ng, less than about 70 ng, less than about 65 ng, less than about 60 ng, less than about 55 ng, less than about 50 ng, less than about 45 ng, less than about 40 ng, less than about 35 ng, less than about 30 ng, less than about 25 ng, less than about 20 ng, less than about 15 ng, less than about 10 ng, or less than about 5 ng. In some embodiments, methods provided herein can be used to the presence or level of ctDNA present in cell-free DNA, where the ctDNA represents 100% of the cell-free DNA. In some embodiments, methods provided herein can be used to detect the presence or level of ctDNA present in cell-free DNA, where the ctDNA represents less than 100% of the cell-free DNA, e.g. about 95%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5%, about 4%, about 3%, about 2%, about 1%, about 0.95%, about 0.90%, about 0.85%, about 0.80%, about 0.75%, about 0.70%, about 0.65%, about 0.60%, about 0.55%, about 0.50%, about 0.45%, about 0.40%, about 0.35%, about 0.30%, about 0.25%, about 0.20%, about 0.15%, about 0.10%, about 0.09%, about 0.08%, about 0.07%, about 0.06%, about 0.05% of the cell-free DNA, or less.

### Identifying the Presence or Levels of TCR Clonotype Levels in a Subject

Provided herein are methods for identifying the presence or level of TCR at least one TCR clonotype (e.g., at least two, at least, three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-five, at least thirty, at least thirty-five, at least forty, at least forty-five, at least fifty, between 1 and 50, between 1 and 45, between 1 and 40, between 1 and 35, between 1 and 30, between 1 and 25, between 1 and 20, between 1 and 15, between 1 and 10, between 1 and 5, between 5 and 10, between 5 and 15, between 5 and 20, between 5 and 25, between 5 and 30, between 5 and 35, between 5 and 40, between 5 and 45, between 5 and 50, between 10 and 15, between 10 and 20, between 10 and 25, between 10 and 30, between 10 and 35, between 10 and 40, between 10 and 45, between 10 and 50, between 15 and 20, between 15 and 25, between 15 and 30, between 15 and 35, between 15 and 40, between 15 and 45, between 15 and 50, between 20 and 25, between 20 and 30, between 20 and 35, between 20 and 40, between 20 and 50, between 25 and 30, between 25 and 50, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 clonotypes) in a subject (e.g., a first level of at least one TCR clonotype at a first time point and/or a second level of at least one TCR clonotype at a second time point).

In some embodiments, the level of at least one TCR clonotype indicates the tumor burden in the subject.

In some embodiments, the biological sample is isolated from subject. Any suitable biological sample that contains cell-free DNA (e.g., cell-free DNA that includes ctDNA) can be used in accordance with any of the variety of methods disclosed herein. For example, the biological sample can include blood, plasma, serum, urine, cerebrospinal fluid, saliva, sputum, broncho-alveolar lavage, bile, lymphatic fluid, cyst fluid, stool, uterine lavage, vaginal fluids, ascites, and combinations thereof. Methods of isolating biological samples from a subject are known to those of ordinary skill in the art.

In some embodiments, detecting the presence or level of at least one TCR clonotype (e.g., at least two, at least, three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-five, at least thirty, at least thirty-five, at least forty, at least forty-five, at least fifty, between 1 and 50, between 1 and 45, between 1 and 40, between 1 and 35, between 1 and 30, between 1 and 25, between 1 and 20, between 1 and 15, between 1 and 10, between 1 and 5, between 5 and 10, between 5 and 15, between 5 and 20, between 5 and 25, between 5 and 30, between 5 and 35, between 5 and 40, between 5 and 45, between 5 and 50, between 10 and 15, between 10 and 20, between 10 and 25, between 10 and 30, between 10 and 35, between 10 and 40, between 10 and 45, between 10 and 50, between 15 and 20, between 15 and 25, between 15 and 30, between 15 and 35, between 15 and 40, between 15 and 45, between 15 and 50, between 20 and 25, between 20 and 30, between 20 and 35, between 20 and 40, between 20 and 50, between 25 and 30, between 25 and 50, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 clonotypes) is performed using one or more of the methods described herein (e.g., a targeted capture method, a next-generation sequencing method, and an array-based method, or any combinations thereof).

In some embodiments, detecting the presence or level of at least one TCR clonotype (e.g., at least two, at least, three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-five, at least thirty, at least thirty-five, at least forty, at least forty-five, at least fifty, between 1 and 50, between 1 and 45, between 1 and 40, between 1 and 35, between 1 and 30, between 1 and 25, between 1 and 20, between 1 and 15, between 1 and 10, between 1 and 5, between 5 and 10, between 5 and 15, between 5 and 20, between 5 and 25, between 5 and 30, between 5 and 35, between 5 and 40, between 5 and 45, between 5 and 50, between 10 and 15, between 10 and 20, between 10 and 25, between 10 and 30, between 10 and 35, between 10 and 40, between 10 and 45, between 10 and 50, between 15 and 20, between 15 and 25, between 15 and 30, between 15 and 35, between 15 and 40, between 15 and 45, between 15 and 50, between 20 and 25, between 20 and 30, between 20 and 35, between 20 and 40, between 20 and 50, between 25 and 30, between 25 and 50, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 clonotypes) is performed using sequencing technology (e.g., a next-generation).

In some embodiments, detecting the presence or level of at least one TCR clonotype is performed using droplet digital PCR (ddPCR). In some embodiments, detecting the presence or level of at least one TCR clonotype is performed using other sequencing technologies, including but not limited to, chain-termination techniques, shotgun techniques, sequencing-by-synthesis methods, methods that utilize microfluidics, other capture technologies, or any of the other sequencing techniques known in the art that are useful for detection of small amounts of DNA in a sample (e.g., TCR clonotype in a cell-free DNA sample).

In some embodiments, detecting the presence or level of at least one TCR clonotype is performed using array-based methods. For example, detecting the presence or level of at least one TCR clonotype can be performed using a DNA microarray. In some embodiments, a DNA microarray can detect the presence or level of at least one TCR clonotype.

Non-limiting examples of array-based methods that can be used in any of the methods described herein, include: a complementary DNA (cDNA) microarray (Kumar et al. (2012) J. Pharm. Bioallied Sci. 4(1): 21-26; Laere et al. (2009) Methods Mol. Biol. 512: 71-98; Mackay et al. (2003) Oncogene 22: 2680-2688; Alizadeh et al. (1996) Nat. Genet. 14: 457-460), an oligonucleotide microarray (Kim et al. (2006) Carcinogenesis 27(3): 392-404; Lodes et al. (2009) PLoS One 4(7): e6229), a bacterial artificial chromosome (BAC) clone chip (Chung et al. (2004) Genome Res. 14(1): 188-196; Thomas et al. (2005) Genome Res. 15(12): 1831-1837), a single-nucleotide polymorphism (SNP) microarray (Mao et al. (2007) Curr. Genomics 8(4): 219-228; Jasmine et al. (2012) PLoS One 7(2): e31968), a microarray-based comparative genomic hybridization array (array-CGH) (Beers and Nederlof (2006) Breast Cancer Res. 8(3): 210; Pinkel et al. (2005) Nat. Genetics 37: S11-S17; Michels et al. (2007) Genet. Med. 9: 574-584), a molecular inversion probe (MIP) assay (Wang et al. (2012) Cancer Genet 205(7-8): 341-55; Lin et al. (2010) BMC Genomics 11: 712). In some embodiments, the cDNA microarray is an Affymetrix microarray (Irizarry (2003) Nucleic Acids Res 31:e15; Dalma-Weiszhausz et al. (2006) Methods Enzymol. 410: 3-28), a NimbleGen microarray (Wei et al. (2008) Nucleic Acids Res 36(9): 2926-2938; Albert et al. (2007) Nat. Methods 4: 903-905), an Agilent microarray (Hughes et al. (2001) Nat. Biotechnol. 19(4): 342-347), or a BeadArray array (Liu et al. (2017) Biosens Bioelectron 92: 596-601). In some embodiments, the oligonucleotide microarray is a DNA tiling array (Mockler and Ecker (2005) Genomics 85(1): 1-15; Bertone et al. (2006) Genome Res 16(2): 271-281). Other suitable array-based methods are known in the art.

### Selecting a Subject for Further Diagnostic Testing

Also provided herein are methods for selecting a subject for further diagnostic testing when an immunotherapy is determined not to be effective in the subject. In some embodiments, methods for selecting a subject for further diagnostic testing include detecting a first level of circulating tumor DNA (ctDNA) and/or a first level of at least one TCR clonotype in a biological sample isolated from the subject at a first time point; detecting a second level of ctDNA and/or a second level of the at least one TCR clonotype in a biological sample obtained from the subject at a second time point, wherein the subject has received at least one dose of an immunotherapy between the first time point and the second time point; and selecting a subject for further diagnostic testing when the immunotherapy is determined to be ineffective (e.g., by comparing differences in the levels of ctDNA and/or TCR clonotypes at the first and second time points).

In some embodiments, the biological sample is isolated from subject. Any suitable biological sample that contains cell-free DNA can be used in accordance with any of the variety of methods disclosed herein. For example, the biological sample can include blood, plasma, serum, urine, cerebrospinal fluid, saliva, sputum, broncho-alveolar lavage, bile, lymphatic fluid, cyst fluid, stool, uterine lavage, vaginal fluids, ascites, and combinations thereof. Methods of isolating biological samples from a subject are known to those of ordinary skill in the art.

In some embodiments, the step of detecting a level of circulating tumor DNA (ctDNA) and/or a level of at least one TCR clonotype is performed using one or more of the methods described herein (e.g., a targeted capture method, a next-generation sequencing method, and an array-based method, or any combinations thereof).

In some embodiments, the diagnostic testing method is a scan. In some embodiments, the scan is a computed tomography (CT), a CT angiography (CTA), a esophagram (a Barium swallom), a Barium enema, a magnetic resonance imaging (MRI), a PET scan, an ultrasound (e.g., an endobronchial ultrasound, an endoscopic ultrasound), an X-ray, a DEXA scan.

In some embodiments, the diagnostic testing method is a physical examination, such as an anoscopy, a bronchoscopy (e.g., an autofluorescence bronchoscopy, a white-light bronchoscopy, a navigational bronchoscopy), a colonoscopy, a digital breast tomosynthesis, an endoscopic retrograde cholangiopancreatography (ERCP), an ensophagogastroduodenoscopy, a mammography, a Pap smear, a pelvic exam, a positron emission tomography and computed tomography (PET-CT) scan.

In some embodiments, the diagnostic testing method is a biopsy (e.g., a bone marrow aspiration, a tissue biopsy). In some embodiments, the biopsy is performed by fine needle aspiration or by surgical excision. In some embodiments, the diagnostic testing methods further includes obtaining a biological sample (e.g., a tissue sample, a urine sample, a blood sample, a check swab, a saliva sample, a mucosal sample (e.g., sputum, bronchial secretion), a nipple aspirate, a secretion or an excretion).

In some embodiments, the diagnostic testing method includes determining the presence of a circulating tumor cell. In some embodiments, the diagnostic testing method includes determining the complete blood cell count (i.e. the percentage and types of immune cells). In some embodiments, the diagnostic testing method is a fecal occult blood test.

For example, a subject selected for further diagnostic testing can also be selected for increased monitoring, in which the subject is administered a diagnostic test at a frequency of twice daily, daily, bi-weekly, weekly, bi-monthly, monthly, quarterly, semi-annually, annually, or any at frequency therein. In some embodiments, a subject selected for further diagnostic testing can also be selected for increased monitoring, in which the subject is administered one or more additional diagnostic tests compared to a subject that has not been selected for further diagnostic testing and increased monitoring.

### Immunotherapy

An immunotherapy can be administered to the patient in methods described herein. The term “immunotherapy” refers to a therapeutic treatment that involves administering to a patient an agent that modulates the immune system. For example, an immunotherapy can increase the expression and/or activity of a regulator of the immune system. In other instances, an immunotherapy can decrease the expression and/or activity of a regulator of the immune system. In some instances, an immunotherapy can recruit and/or enhance the activity of an immune cell. An example of an immunotherapy is a therapeutic treatment that involves administering at least one, e.g., two or more, immune checkpoint inhibitors. Exemplary immune checkpoint inhibitors useful in the presently-described methods are CTLA-4 inhibitors, PD-1 inhibitors or PD-L1 inhibitors, or combinations thereof.

The immunotherapy can be a cellular immunotherapy (e.g., adoptive T-cell therapy, dendritic cell therapy, natural killer cell therapy). For example, the cellular immunotherapy can be sipuleucel-T (APC8015; Provenge™; Plosker (2011) Drugs 71(1): 101-108). In some instances, the cellular immunotherapy includes cells that express a chimeric antigen receptor (CAR). In some instances, the cellular immunotherapy can be a CAR-T cell therapy, e.g., tisagenlecleucel (Kymriah™).

Immunotherapy can be, e.g., an antibody therapy (e.g., a monoclonal antibody, a conjugated antibody). Exemplary antibody therapies are bevacizumab (Mvasti™, Avastin®), trastuzumab (Herceptin®), avelumab (Bavencio®), rituximab (MabThera™, Rituxan®), edrecolomab (Panorex), daratumuab (Darzalex®), olaratumab (Lartruvo™), ofatumumab (Arzerra®), alemtuzumab (Campath®), cetuximab (Erbitux®), oregovomab, pembrolizumab (Keytruda®), dinutiximab (Unituxin®), obinutuzumab (Gazyva®), tremelimumab (CP-675,206), ramucirumab (Cyramza®), ublituximab (TG-1101), panitumumab (Vectibix®), elotuzumab (Empliciti™), avelumab (Bavencio®), necitumumab (Portrazza™), cirmtuzumab (UC-961), ibritumomab (Zevalin®), isatuximab (SAR650984), nimotuzumab, fresolimumab (GC1008), lirilumab (INN), mogamulizumab (Poteligeo®), ficlatuzumab (AV-299), denosumab (Xgeva®), ganitumab, urelumab, pidilizumab or amatuximab.

An immunotherapy described herein can involve administering an antibody-drug conjugate to a patient. The antibody-drug conjugate can be, e.g., gemtuzumab ozogamicin (Mylotarg™), inotuzumab ozogamicin (Besponsa®), brentuximab vedotin (Adcetris®), ado-trastuzumab emtansine (TDM-1; Kadcyla®), mirvetuximab soravtansine (IMGN853) or anetumab ravtansine.

In some instances, the immunotherapy includes blinatumomab (AMG103; Blincyto®) or midostaurin (Rydapt).

An immunotherapy can include administering to the patient a toxin. For example, the immunotherapy can including administering denileukin diftitox (Ontak®).

In some instances, the immunotherapy can be a cytokine therapy. The cytokine therapy can be, e.g., an interleukin 2 (IL-2) therapy, an interferon alpha (IFN-α) therapy, a granulocyte colony stimulating factor (G-CSF) therapy, an interleukin 12 (IL-12) therapy, an interleukin 15 (IL-15) therapy, an interleukin 7 (IL-7) therapy or an erythropoietin-alpha (EPO) therapy. In some embodiments, the IL-2 therapy is aldesleukin (Proleukin®). In some embodiments, the IFN-α therapy is IntronA® (Roferon-A®). In some embodiments, the G-CSF therapy is filgrastim (Neupogen®).

In some instances, the immunotherapy is an immune checkpoint inhibitor. For example, the immunotherapy can include administering one or more immune checkpoint inhibitors. In some embodiments, the immune checkpoint inhibitor is a CTLA-4 inhibitor, a PD-1 inhibitor or a PD-L1 inhibitor. An exemplary CTLA-4 inhibitor would be, e.g., ipilimumab (Yervoy®) or tremelimumab (CP-675,206). In some embodiments, the PD-1 inhibitor is pembrolizumab (Keytruda®) or nivolumab (Opdivo®). In some embodiments, the PD-L1 inhibitor is atezolizumab (Tecentriq®), avelumab (Bavencio®) or durvalumab (Imfinzi™).

In some instances, the immunotherapy is mRNA-based immunotherapy. For example, the mRNA-based immunotherapy can be CV9104 (see, e.g., Rausch et al. (2014) Human Vaccin Immunother 10(11): 3146-52; and Kubler et al. (2015) J. Immunother Cancer 3:26).

In some instances, the immunotherapy can involve bacillus Calmette-Guerin (BCG) therapy.

In some instances, the immunotherapy can be an oncolytic virus therapy. For example, the oncolytic virus therapy can involve administering talimogene alherparepvec (T-VEC; Imlygic®).

In some instances, the immunotherapy is a cancer vaccine, e.g., a human papillomavirus (HPV) vaccine. For example, an HPV vaccine can be Gardasil®, Gardasil9® or Cervarix®. In some instances, the cancer vaccine is a hepatitis B virus (HBV) vaccine. In some embodiments, the HBV vaccine is Engerix-B®, Recombivax HB® or GI-13020 (Tarmogen®). In some embodiments, the cancer vaccine is Twinrix® or Pediarix®. In some embodiments, the cancer vaccine is BiovaxID®, Oncophage®, GVAX, ADXS11-001, ALVAC-CEA, PROSTVAC®, Rindopepimut®, CimaVax-EGF, lapuleucel-T (APC8024; Neuvenge™), GRNVAC1, GRNVAC2, GRN-1201, hepcortespenlisimut-L (Hepko-V5), DCVAX®, SCIB1, BMT CTN 1401, PrCa VBIR, PANVAC, ProstAtak®, DPX-Survivac, or viagenpumatucel-L (HS-110). The immunotherapy can involve, e.g., administering a peptide vaccine. For example, the peptide vaccine can be nelipepimut-S(E75) (NeuVax™), IMA901, or SurVaxM (SVN53-67). In some instances, the cancer vaccine is an immunogenic personal neoantigen vaccine (see, e.g., Ott et al. (2017) Nature 547: 217-221; Sahin et al. (2017) Nature 547: 222-226). In some embodiments, the cancer vaccine is RGSH4K, or NEO-PV-01. In some embodiments, the cancer vaccine is a DNA-based vaccine. In some embodiments, the DNA-based vaccine is a mammaglobin-A DNA vaccine (see, e.g., Kim et al. (2016) Oncolmmunology 5(2): e1069940).

### Therapeutic Interventions

In some embodiments, when an immunotherapy is determined not to be effective in a subject (e.g., using any of the variety of methods disclosed herein), a therapeutic intervention (e.g., a therapeutic intervention that is different from the ineffective immunotherapy) can be administered to the subject. Exemplary therapeutic interventions include, without limitation, adoptive T cell therapy (e.g., chimeric antigen receptors and/or T cells having wild-type or modified T cell receptors), radiation therapy, surgery (e.g., surgical resection), and administration of one or more chemotherapeutic agents, administration of immune checkpoint inhibitors, targeted therapies such as kinase inhibitors (e.g., kinase inhibitors that target a particular genetic lesion, such as a translocation or mutation), signal transduction inhibitors, bispecific antibodies, and/or monoclonal antibodies. Such therapeutic interventions can be administered alone or in combination.

In some embodiments, the therapeutic intervention can include an immune checkpoint inhibitor (e.g., a single immune checkpoint inhibitor or a combination of immune checkpoint inhibitors). Non-limiting examples of immune checkpoint inhibitors include nivolumab (Opdivo), pembrolizumab (Keytruda), atezolizumab (tecentriq), avelumab (bavencio), durvalumab (imfinzi), ipilimumab (yervoy). See, e.g., Pardoll (2012) Nat. Rev Cancer 12: 252-264; Sun et al. (2017) Eur Rev Med Pharmacol Sci 21(6): 1198-1205; Hamanishi et al. (2015) J. Clin. Oncol. 33(34): 4015-22; Brahmer et al. (2012) N Engl J Med 366(26): 2455-65; Ricciuti et al. (2017) J. Thorac Oncol. 12(5): e51-e55; Ellis et al. (2017) Clin Lung Cancer pii: 51525-7304(17)30043-8; Zou and Awad (2017) Ann Oncol 28(4): 685-687; Sorscher (2017) N Engl J Med 376(10: 996-7; Hui et al. (2017) Ann Oncol 28(4): 874-881; Vansteenkiste et al. (2017) Expert Opin Biol Ther 17(6): 781-789; Hellmann et al. (2017) Lancet Oncol. 18(1): 31-41; Chen (2017) J. Chin Med Assoc 80(1): 7-14.

In some embodiments, a therapeutic intervention is adoptive T cell therapy (e.g., chimeric antigen receptors and/or T cells having wild-type or modified T cell receptors). See, e.g., Rosenberg and Restifo (2015) Science 348(6230): 62-68; Chang and Chen (2017) Trends Mol Med 23(5): 430-450; Yee and Lizee (2016) Cancer J. 23(2): 144-148; Chen et al. (2016) Oncoimmunology 6(2): e1273302; US 2016/0194404; US 2014/0050788; US 2014/0271635; U.S. Pat. No. 9,233,125; incorporated by reference in their entirety herein.

In some embodiments, a therapeutic intervention is a chemotherapeutic agent. Non-limiting examples of chemotherapeutic agents include: amsacrine, azacitidine, axathioprine, bevacizumab (or an antigen-binding fragment thereof), bleomycin, busulfan, carboplatin, capecitabine, chlorambucil, cisplatin, cyclophosphamide, cytarabine, dacarbazine, daunorubicin, docetaxel, doxifluridine, doxorubicin, epirubicin, erlotinib hydrochlorides, etoposide, fiudarabine, floxuridine, fludarabine, fluorouracil, gemcitabine, hydroxyurea, idarubicin, ifosfamide, irinotecan, lomustine, mechlorethamine, melphalan, mercaptopurine, methotrxate, mitomycin, mitoxantrone, oxaliplatin, paclitaxel, pemetrexed, procarbazine, all-trans retinoic acid, streptozocin, tafluposide, temozolomide, teniposide, tioguanine, topotecan, uramustine, valrubicin, vinblastine, vincristine, vindesine, vinorelbine, and combinations thereof. Additional examples of anti-cancer therapies are known in the art; see, e.g. the guidelines for therapy from the American Society of Clinical Oncology (ASCO), European Society for Medical Oncology (ESMO), or National Comprehensive Cancer Network (NCCN).

In some embodiments, the therapeutic intervention can result in an early onset of remission of a cancer in a subject. In some embodiments, the therapeutic intervention can result in an increase in the time of remission of a cancer in a subject. In some embodiments, the therapeutic intervention can result in an increase in the time of survival of a subject. In some embodiments, the therapeutic intervention can result in decreasing the size of a solid primary tumor in a subject. In some embodiments, the therapeutic intervention can result in decreasing the volume of a solid primary tumor in a subject. In some embodiments, the therapeutic intervention can result in decreasing the size of a metastasis in a subject. In some embodiments, the therapeutic intervention can result in decreasing the volume of a metastasis in a subject. In some embodiments, the therapeutic intervention can result in decreasing the tumor burden in a subject.

In some embodiments, the therapeutic intervention can result in improving the prognosis of a subject. In some embodiments, the therapeutic intervention can result in decreasing the risk of developing a metastasis in a subject. In some embodiments, the therapeutic intervention can result in decreasing the risk of developing an additional metastasis in a subject. In some embodiments, the therapeutic intervention can result in decreasing cancer cell migration in a subject. In some embodiments, the therapeutic intervention can result in decreasing cancer cell invasion in a subject. In some embodiments, the therapeutic intervention can result in a decrease in the time of hospitalization of a subject. In some embodiments, the therapeutic intervention can result in a decrease of the presence of cancer stem cells within a tumor in a subject.

In some embodiments, the therapeutic intervention can result in an increase in immune cell infiltration within the tumor microenvironment in a subject. In some embodiments, the therapeutic intervention can result in altering the immune cell composition within the tumor microenvironment of a tumor in a subject. In some embodiments, the therapeutic intervention can result in modulating a previously-immunosuppressive tumor microenvironment into an immunogenic, inflammatory tumor microenvironment. In some embodiments, the therapeutic intervention can result in a reversal of the immunosuppressive tumor microenvironment in a subject.

In some embodiments, the therapeutic intervention can halt tumor progression in a subject. In some embodiments, the therapeutic intervention can delay tumor progression in a subject. In some embodiments, the therapeutic intervention can inhibit tumor progression in a subject. In some embodiments, the therapeutic intervention can inhibit immune checkpoint pathways of a tumor in a subject. In some embodiments, the therapeutic intervention can immuno-modulate the tumor microenvironment of a tumor in a subject. In some embodiments, the therapeutic intervention can immuno-modulate the tumor macroenvironment of a tumor in a subject.

In some embodiments of any of the methods described herein, the subject can be administered a single or multiple doses (e.g., two, three, four, five, six, seven, eight, nine, or ten doses) of any of the therapeutic interventions described herein.

In some embodiments of any of the methods described herein, the method can further include administering one or more therapeutic interventions.

As used herein, the terms “in combination” or “combination therapy” describe any concurrent or parallel treatment with at least two distinct therapeutic agents, e.g., administration of any of at least two therapeutic interventions. In some embodiments of any of the methods described herein, the one or more therapeutic interventions are administered sequentially or simultaneously to the subject after the cancer cell has been detected. For example, the one or more therapeutic interventions can include chemotherapeutic agents, anti-angiogenic agents, apoptosis-inducing agents, surgical resection, and radiotherapy. In some embodiments, combined therapy is an epigenetic therapy (e.g., any of the epigenetic therapies described herein) and an immunotherapy (e.g., any of the immunotherapies described herein). In some embodiments, the combined therapy is 5-AZA and an immune checkpoint inhibitor (e.g., anti-PD1 and/or anti-CTLA-4 inhibitor) (Kim (2014) PNAS 111(32): 11774-1179; Wang (2015) Cancer Immunol. Res. 3(9): 1030-1041; Juergens et al. (2011) Cancer Discov 1(7): 598-607).

### Cancers

A subject according to any of the methods described herein can have a cancer that includes, without limitation, lung cancer (e.g., small cell lung carcinoma or non-small cell lung carcinoma), papillary thyroid cancer, medullary thyroid cancer, differentiated thyroid cancer, recurrent thyroid cancer, refractory differentiated thyroid cancer, lung adenocarcinoma, bronchioles lung cell carcinoma, multiple endocrine neoplasia type 2A or 2B (MEN2A or MEN2B, respectively), pheochromocytoma, parathyroid hyperplasia, breast cancer, colorectal cancer (e.g., metastatic colorectal cancer), papillary renal cell carcinoma, ganglioneuromatosis of the gastroenteric mucosa, inflammatory myofibroblastic tumor, or cervical cancer, acute lymphoblastic leukemia (ALL), acute myeloid leukemia (AML), cancer in adolescents, adrenal cancer, adrenocortical carcinoma, anal cancer, appendix cancer, astrocytoma, atypical teratoid/rhabdoid tumor, basal cell carcinoma, bile duct cancer, bladder cancer, bone cancer, brain stem glioma, brain tumor, breast cancer, bronchial tumor, Burkitt lymphoma, carcinoid tumor, unknown primary carcinoma, cardiac tumors, cervical cancer, childhood cancers, chordoma, chronic lymphocytic leukemia (CLL), chronic myelogenous leukemia (CML), chronic myeloproliferative neoplasms, colon cancer, colorectal cancer, craniopharyngioma, cutaneous T-cell lymphoma, bile duct cancer, ductal carcinoma in situ, embryonal tumors, endometrial cancer, ependymoma, esophageal cancer, esthesioneuroblastoma, Ewing sarcoma, extracranial germ cell tumor, extragonadal germ cell tumor, extrahepatic bile duct cancer, eye cancer, fallopian tube cancer, fibrous histiocytoma of bone, gallbladder cancer, gastric cancer, gastrointestinal carcinoid tumor, gastrointestinal stromal tumors (GIST), germ cell tumor, gestational trophoblastic disease, glioma, hairy cell tumor, hairy cell leukemia, head and neck cancer, heart cancer, hepatocellular cancer, histiocytosis, Hodgkin's lymphoma, hypopharyngeal cancer, intraocular melanoma, islet cell tumors, pancreatic neuroendocrine tumors, Kaposi sarcoma, kidney cancer, Langerhans cell histiocytosis, laryngeal cancer, leukemia, lip and oral cavity cancer, liver cancer, lung cancer, lymphoma, macroglobulinemia, malignant fibrous histiocytoma of bone, osteocarcinoma, melanoma, Merkel cell carcinoma, mesothelioma, metastatic squamous neck cancer, midline tract carcinoma, mouth cancer, multiple endocrine neoplasia syndromes, multiple myeloma, mycosis fungoides, myelodysplastic syndromes, myelodysplastic/myeloproliferative neoplasms, myelogenous leukemia, myeloid leukemia, multiple myeloma, myeloproliferative neoplasms, nasal cavity and paranasal sinus cancer, nasopharyngeal cancer, neuroblastoma, non-Hodgkin's lymphoma, non-small cell lung cancer, oral cancer, oral cavity cancer, lip cancer, oropharyngeal cancer, osteosarcoma, ovarian cancer, pancreatic cancer, papillomatosis, paraganglioma, paranasal sinus and nasal cavity cancer, parathyroid cancer, penile cancer, pharyngeal cancer, pheochromosytoma, pituitary cancer, plasma cell neoplasm, pleuropulmonary blastoma, pregnancy and breast cancer, primary central nervous system lymphoma, primary peritoneal cancer, prostate cancer, rectal cancer, renal cell cancer, retinoblastoma, rhabdomyosarcoma, salivary gland cancer, sarcoma, Sezary syndrome, skin cancer, small cell lung cancer, small intestine cancer, soft tissue sarcoma, squamous cell carcinoma, squamous neck cancer, stomach cancer, T-cell lymphoma, testicular cancer, throat cancer, thymoma and thymic carcinoma, thyroid cancer, transitional cell cancer of the renal pelvis and ureter, unknown primary carcinoma, urethral cancer, uterine cancer, uterine sarcoma, vaginal cancer, vulvar cancer, Waldenstrom Macroglobulinemia, and Wilms' tumor.

In some embodiments of any of the methods described herein, the subject has non-small cell lung cancer, melanoma, ovarian cancer, colorectal cancer, breast cancer and prostate cancer. In some embodiments of any of the methods described herein, the subject has a head and neck cancer, a central nervous system cancer, a lung cancer, a mesothelioma, an esophageal cancer, a gastric cancer, a gall bladder cancer, a liver cancer, a pancreatic cancer, a melanoma, an ovarian cancer, a small intestine cancer, a colorectal cancer, a breast cancer, a sarcoma, a kidney cancer, a bladder cancer, an uterine cancer, a cervical cancer, and a prostate cancer.

Various embodiments of such cancers, as therapeutic interventions appropriate to treat such cancers, are described herein.

### Colorectal Cancer

In some embodiments wherein the subject has colorectal cancer, the subject may have hereditary colorectal cancer. In some embodiments, the subject has polyposis (e.g., familial adenomatous polyposis (FAP) or attenuated FAP (AFAP) (Half et al. (2009) Orphanet J Rare Dis. 4:22; Knudsen et al. (2003) Fam Cancer 2:43-55). In some embodiments, the subject has a mutation in an adenomatosis polyposis coli (APC) gene and/or a mutY DNA glycosylase (MYH) gene (Theodoratou et al. (2010) Br. J. Cancer 103: 1875-1884). In some embodiments, the subject has hereditary nonpolyposis colorectal cancer (HNPCC; also known as Lynch Syndrome) (Marra et al. (1995) J. Natl. Cancer Inst 87: 1114-1135). In some embodiments, the subject has a mutation in a DNA mismatch repair gene (e.g., mutL homolog 1 (MLH1), mutS homolog 2 (MSH2), mutS homolog 6 (MSH6) and/or PMS1 homolog 2 (PMS2)). In some embodiments, the subject has a mutation in an epithelial cell adhesion molecule (EPCAM) gene. In some embodiments, the subject has a mutation in an axin-related protein 2 (AXIN2) gene (Lammi et al. (2004) Am. J. Hum. Genet. 74: 1043-1050). In some embodiments wherein a colorectal cancer cell has been detected in the subject, the subject has oligopolyposis, juvenile polyposis syndrome, Cowden syndromw, Peutz-Jeghers syndrome (Giardiello et al. (2006) Clin. Gastroenterol. Hepatol. 4:408-415), or serrated polyposis syndrome (Torlakovic et al. (1996) Gastroenterology 110: 748-755). In some embodiments, the subject has hereditary mixed polyposis syndrome (Whitelaw et al. (1997) Gastroenterology 112: 327-334; Tomlinson et al. (1999) Gastronenterology 116: 789-795).

In some embodiments wherein the subject has colorectal cancer, the subject has a colorectal cancer that has at least one mutation in a gene selected from the group consisting of: adenomatosis polyposis coli (APC), mutY DNA glycosylase (MYH), mutL homolog 1 (MLH1), mutS homolog 2 (MSH2), mutS homolog 6 (MSH6), PMS1 homolog 2 (PMS2), epithelial cell adhesion molecule (EPCAM), DNA polymerase epsilon (POLE), DNA polymerase delta 1 (POLD1), nth like DNA glycosylase 1 (NTHL1), bone morphogenetic protein receptor type 1A (BMPR1A), SMAD family member 4 (SMAD4), phosphatase and tensin homolog (PTEN), serine/threonine kinase 11 (LKBJ, STKJJ), transforming growth factor beta receptor 2 (TGFβRII), phosphatidylinositol-4,5-biphosphate-3-kinase catalytic subunit alpha (PIK3CA), tumor protein p53 (TP53), epidermal growth factor receptor (EGFR), B-raf proto-oncogene (BRAT), phosphatidylinositol-4,5-biphosphate-3-kinase (PI3K), A-T rich interaction domain 1A (ARID1A), sex determining region Y-bod 9 (SOX9), erb-b2 receptor tyrosine kinase 2 (ERBB2), insulin like growth factor 2 (IGF2), APC membrane recruitment protein (FAM123B; AMER1), neuron navigator 2 (NAV2), vacuolar protein sorting 72 homolog (TCFL1; VPS72), N-Ras proto-oncogene (NRAS), and combinations thereof. See, e.g., Armaghany et al. (2012) Gastrointest. Cancer Res. 5(1): 19-27; Bulow et al. (2004) Gut 53: 381-386; Zeichner et al. (2012) Clin. Med. Insights Oncol. 6: 315-323; The Cancer Genomic Atlas Network (2012) Nature 487: 330-337; Kemp et al. (2004) Hum. Mol. Genet. 13(suppl_2: R177-R185; Zouhairi et al. (2011) Gastrointest Cancer Res 4(1): 15-21.

In some embodiments, the subject has a genetic mutation that can result in activation of a proto-oncogene (e.g., KRAS). In some embodiments, the subject has a genetic mutation that can result in inactivation of a tumor suppressor gene (e.g., 1, 2, 3, 4, 5, 6, at least 1, at least 2 or at least 3 tumor suppressor genes). In some embodiments, at least three tumor suppressor genes are inactivated (e.g., APC, TP53, and loss of heterozygosity of long arm of chromosome 18). In some embodiments, the subject has a genetic mutation in a gene involved in the APC/Wnt/β-catenin pathway. In some embodiments, the genetic mutation is a nonsense mutation or a frameshift mutation, thereby resulting in a truncated protein. In some embodiments, the genetic mutation causes microsatellite instability, epigenetic instability and/or aberrant CpG methylation.

In some embodiments of any of the methods described herein wherein the subject has previously been diagnosed with colorectal cancer, the subject is administered an additional therapeutic intervention that specifically targets the genetic modifications present in the subject's colorectal cancer. In some embodiments, the subject was previously administered an anti-EGFR monoclonal antibody (e.g., cetuximab or panitumumab) (Cunningham et al. (2004) N. Engl. J. Med. 351(4): 337-345). In some embodiments, the therapeutic invention is an antiangiogenic agent. In some embodiments, the antiangiogenic agent is bevacizumab (Avastin) (Hurwitz et al. (2004) N. Engl. J. Med. 350: 2335-2342). In some embodiments, the antiangiogenic agent is a VEGF inhibitor (e.g., aflibercept (Tang et al. (2008) J. Clin. Oncol 26 (May 20 suppl; abstr 4027); vatalanib (PTK/ZK222584; Hecht et al. (2005) ASCO Annual Meeting Proceedings J. Clin. Oncol. 23: 16S (abstr. LBA3)); sunitinib (Saltz et al. (2007) J. Clin. Oncol. 25: 4793-4799); AZD2171 (Rosen et al. (2007) J. Clin. Oncol. 25: 2369-76); AMG 706 (Drevis et al. (2007) 25: 3045-2054)).

Non-limiting examples of chemotherapy treatments that can be used in a subject with colorectal cancer include: 5-FU, leucovorin, oxaliplatin (Eloxatin), capecitabine, celecoxib and sulindac. In some embodiments, a combination of chemotherapeutic agents is used, e.g., FOLFOX (5-FU, leucovorin and oxaliplatin), FOLFIRI (leucovorin, 5-FU and irinotecan (Camptosar), CapeOx (capecitabine (Xeloda) and oxaliplatin). In some embodiments, the therapeutic intervention is a mammalian target of rapamycin (mTOR) inhibitor (e.g., a rapamycin analog (Kesmodel et al. (2007) Gastrointestinal Cancers Symposium (abstr 234)); RAD-001 (Tabernero et al. (2008) J. Clin. Oncol. 26: 1603-1610). In some embodiments, the therapeutic intervention is a protein kinase C antagonist (e.g., enzastaurin (Camidge et al. (2008) Anticancer Drugs 19:77-84, Resta et al. (2008) J. Clin. Oncol. 26 (May 20 suppl) (abstr 3529)). In some embodiments, the therapeutic intervention is an inhibitor of nonreceptor tyrosine kinase Src (e.g., AZ0530 (Tabernero et al. (2007) J. Clin. Oncol. 25: 18S (abstr 3520))). In some embodiments, the therapeutic intervention is an inhibitor of kinesin spindle protein (KSP) (e.g., ispinesib (SB-715992) (Chu et al. (2004) J. Clin. Oncol. 22:14S (abstr 2078), Burris et al. (2004) J. Clin. Oncol. 22: 128 (abstr 2004))).

In some embodiments, the therapeutic intervention is surgery (e.g., polypectomy, partial colectomy, colectomy or diverting colostomy). In some embodiments, adjuvant chemotherapy is further administered to the subject after surgery (e.g., polypectomy or partial colectomy). In some embodiments, the therapeutic intervention is a prophylactic surgery (e.g., colectomy). In some embodiments, a cancer may be removed by ablation or embolization.

### Ovarian Cancer

In some embodiments of any of the methods described herein, the subject may have hereditary ovarian cancer (Petrucelli et al. (2010) Gen. Med 12:245-259). In some embodiments, the subject has another genetic condition that may cause ovarian cancer (e.g., Lynch syndrome, Peutz-Jeghers syndrome, nevoid basal cell carcinoma syndrome (NBCCS; also known as Gorlin syndrome), Li-Fraumeni syndrome or Ataxia-Telangiecstasia (Cancer.Net). In some embodiments, the subject may have an invasive epithelial ovarian cancer, an epithelial tumor of low malignant potential (also known as an atypical proliferating tumor or a borderline tumor), a germ cell tumor of the ovary (e.g. a malignant germ cell tumor, a dysgerminoma, an immature teratoma) or a stromal tumor of the ovary.

In some embodiments, the subject's ovarian cancer was caused by a somatic mutation in a gene. In some embodiments, the subject has a mutation in a gene selected from the group consisting of: tumor protein p53 (TP53), breast cancer 1 (BRCA1), breast cancer 2 (BRCA2), mutL homolog 1 (MLH1), mutS homolog 2 (MSH2), AKT serine/threonine kinase 1 (AKT1), BRAC1 associated ring domain 1 (BARD1), BRAC1 interacting protein C-terminal helicase 1 (GRIP1), epithelial cadherin 1 (CDH1), checkpoint kinase 2 (CHEK2), catenin beta 1 (CTNNB1), MRE11 homolog (MRE11), mutS homolog 6 (MSH6), nibrin (NBN), opiod binding protein/cell adhesion molecule like (OPCML), partner and localizer of BRCA2 (PALB2), phosphatidylinositol-4,5-biphosphate-3-kinase catalytic subunit alpha (PIK3CA), PMS1 homolog 2 (PMS2), parkin RBR E3 ubiquitin protein ligase (PRKN), RAD50 double strand break repair protein (RAD50), RAD51 recombinase (RADS1), serine/threonine kinase 11 (LKBJ, STK11), neurofibromin (NF1), retinoblastoma 1 (RB1), cyclin dependent kinase 12 (CDK12), and combinations thereof. See, e.g., Kurman et al. (2011) Hum. Pathol. 42(7): 918-31; Nakayama et al. (2006) Cancer Biol. Ther. 5(7): 779-785; Singer et al. (2003) J. Natl Cancer Inst 95(6): 484-6; Kuo et al. (2009) Am. J. Pathol. 174(5): 1597-601; Gemignani et al. (2003) Gynecol. Oncol. 90(2): 378-81; Levine et al. (2005) Clin. Cancer Res. 11(8): 2875-8; Wang et al. (2005) Hum. Mutat. 25(3): 322; Landen et al. (2008) J. Clin. Oncol. 26(6): 995-1005; Ramus et al. (2015) j Natl Cancer Inst 107(11).

In some embodiments of any of the methods described herein, the additional therapeutic intervention is chemotherapy (e.g., any of the platinum-based chemotherapeutic agents described herein (e.g., cisplatin, carboplatin), or a taxane (e.g., placitaxel (Taxol®) or docetaxel (Taxotere®). In some embodiments, the chemotherapeutic agent is an albumin-bound paclitaxel (nap-paclitaxel, Abraxane®), altretamine (Hexalen®), capecitabine (Xeloda®), cyclophosphamide (Cytoxan®), etoposide(VP-16), gemcitabine (Gemzar®), ifosfamide (Ifex®), irinotecan (CPT-11, Camptosar®), liposomal doxorubicin (Doxil®), melphalan, pemetrexed (Alimta®), topotecan, or vinorelbine (Navelbine®). In some embodiments, the therapeutic intervention is a combination of chemotherapeutic agents (e.g., paclitaxel, ifosfamide, and cisplatin; vinblastine, ifosfamide and cisplatin; etoposide, ifosfamide and cisplatin).

In some embodiments, the therapeutic intervention is an epigenetic therapy (see, e.g., Smith et al. (2017) Gynecol. Oncol. Rep. 20: 81-86). In some embodiments, the epigenetic therapy is a DNA methyltransferase (DNMT) inhibitor (e.g., 5-azacytidine (5-AZA), decitabine (5-aza-2′-deoxycytidine) (Fu et al. (2011) Cancer 117(8): 1661-1669; Falchook et al. (2013) Investig. New Drugs 31(5): 1192-1200; Matei et al. (2012) Cancer Res. 72(9): 2197-2205). In some embodiments, the DNMT1 inhibitor is NY-ESO-1 (Odunsi et al. (2014) Cancer Immunol. Res. 2(1): 37-49). In some embodiments, the epigenetic therapy is a histone deacetylase (HDAC) inhibitor. In some embodiments, the HDAC inhibitor is vorinostat (Modesitt (2008) 109(2): 182-186) or belinostat (Mackay et al. (2010) Eur. J. Cancer 46(9): 1573-1579). In some embodiments, the HDAC inhibitor is given in combination with a chemotherapeutic agent (e.g., carboplatin (paraplatin), cisplatin, paclitaxel or docetaxel (taxotere)) (Mendivil (2013) Int. J. Gynecol. Cancer 23(3): 533-539; Dizon (2012) Gynecol. Oncol. 125(2): 367-371; Dizon (2012) Int J. Gynecol. Cancer 23(3): 533-539).

In some embodiments, the therapeutic intervention is an anti-angiogenic agent (e.g., bevacizumab).

In some embodiments, the therapeutic intervention is a poly (ADP-ribose) polymerase (PARP)-1 and/or PARP-2 inhibitor. In some embodiments, the PARP-1 and PARP-2 inhibitor is niraparib (zejula) (Scott (2017) Drugs doiL10.1007/s40265-017-0752). In some embodiments, the PARP inhibitor is olaparib (lynparza) or rucaparib (rubraca).

In some embodiments, the therapeutic intervention is a hormone (e.g., a luteinizing-hormone-releasing hormone (LHRH) agonist). In some embodiments, the LHRH agonist is goserelin (Zoladex®) or leuprolide (Lupron®). In some embodiments, the therapeutic intervention is an anti-estrogen compound (e.g., tamoxifen). In some embodiments, the therapeutic intervention is an aromatase inhibitor (e.g., letrozole (Femara®), anastrozole (Arimidex®) or exemestane (Aromasin®).

In some embodiments, the therapeutic intervention is surgery (e.g., debulking of the tumor mass, a hysterectomy, a bilateral salpingo-oophorectomy, an omentectomy). The term “debulking” refers to surgical removal of almost the entire tumor (“optimally debulked”). In some embodiments, debulking can include removing a portion of the bladder, the spleen, the gallbladder, the stomach, the liver, and/or pancreas. In some embodiments, adjuvant chemotherapy is further administered to the subject after surgery (e.g., debulking of the tumor mass, a hysterectomy, a bilateral salpingo-oophorectomy, an omentectomy). In some embodiments, adjuvant chemotherapy is administered intra-abdominally (intraperitoneally). In some embodiments, the therapeutic intervention is a prophylactic surgery (e.g., a hysterectomy). In some embodiments, a paracentesis is performed to remove ascites.

In some embodiments, the therapeutic intervention is radiation therapy. In some embodiments, the radiation therapy is external beam radiation therapy, brachytherapy or a use of radioactive phosphorus.

### Lung Cancer

In some embodiments of any of the methods described herein, the subject may have hereditary lung cancer (Gazdar et al. (2014) J. Thorac. Oncol. 9(4): 456-63). In some embodiments, the subject has non-small cell-lung cancer (NSCLC) or small cell lung cancer (SCLC).

In some embodiments, the subject's lung cancer was caused by a somatic mutation in a gene. In some embodiments, the subject has a mutation in a gene selected from the group consisting of: ARID1A, AKT, anaplastic lymphoma kinase (ALK), BRAF, cyclin dependent kinase inhibitor 2 (CDKN2A), discoidin domain receptor tyrosine kinase 2 (DDR2), epidermal growth factor receptor (EGFR), fibroblast growth factor receptor 1 (FGFR1), HER2/ERBB2, kelch like ECH associated protein 1 (KEAP1) (Singh et al. (2006) PLoS Med 3: e420), KRAS proto-oncogene (KRAS), MAP kinase/ERK kinase 1 (MEK1), MET proto-oncogene (MET), MAX gene associated (MGA), myelocytomatosis oncogene (MYC), NFL NRAS, neutrophophic receptor tyrosine kinase 1 (NTRK1), PTEN, PIK3CA, RB1, RNA binding motif protein 10 (RBM10), ret proto-oncogene (RET), Ras like without CAAX 1 (RIT1) (Berger et al. (2014) Oncogene), Ros proto-oncogene (ROS1), STE domain containing 2 (SETD2), SWI/SNF related matrix associated actin dependent regulator of chromatin, subfamily A, member 4 (SMARCA4) (Medina et al. (2008) Hum. Mutat. 29: 617-622), (SOX2) (Rudin et al. (2012) Nature Genet. 44: 111-1116), LKB1 (STK11) (Sanchez-Cespedees et al. (2002) Cancer Res. 62: 3659-3662), TP53 (Takahashi et al. (1989) Science 246: 491-494), U2 small nuclear RNA auxillary factor 1 (U2AF1), and combinations thereof. See e.g., The Cancer Genome Atlas Research Network (2014) Nature 511: 543-550; Ding et al. (2008) Nature 1069-1075; The Cancer Genome Atlas Research Network (2012) Nature 489: 519-525; Seo et al. (2012) Genome Res. 22: 2109-2119; El-Telbany and Ma (2012) Genes Cancer 3(7-8): 467-480; Marks et al. (2008) Cancer Res. 68: 5524-5528; De Braud et al. (2014) J. Clin. Oncol. 32: 2502; Rothschild (2015) Cancers 7: 930949.

In some embodiments, a copy number variation or an oncogenic chromosomal gene rearrangement (e.g., oncogenic chromosomal translocation) is detected in a lung cancer cell. Non-limiting examples of oncogenic chromosomal translocation found in lung cancer include: EML4-ALK, TFG-ALK, KIF5B-ALK, KLC1-ALK, PTPN3-ALK, TPR-ALK, HIP1-ALK, STRN-ALK, DCTN1-ALK, SQSTM1-ALK, BIRC6-ALK, RET-PTC1, KIF4B-RET, CCDCl₆-RET and NCOA4-RET. See, e.g., Iyevleva et al. (2015) Cancer Lett. 362(1): 116-121; Wang et al. (2012) J. Clin Oncol. 30: 4352-9

In some embodiments, the therapeutic intervention is an anti-angiogenic agent (e.g., bevacizumab (avastin), ramucirumab (cyramza)).

In some embodiments, the therapeutic intervention is a targeted drug therapy. In some embodiments, the targeted drug therapy is an EGFR inhibitor (e.g., erlotinib (tarceva), afatinib (gilotrif), gefitinib (iressa), necitumumab (portrazza), cetuximab, osimertinib (AZD9291, Tagrisso), rociletinib (CO-1686), HM61713 (BI 1482694), ASP8273, EGF816, PF-06747775). See, e.g., Wang et al. (2016) J. Hematol Oncol 9:34; Cross et al. (2014) Cancer Discov. 4(9): 1046-61; Walter et al. (2013) Cancer Discov 3(12): 1404-15; Park et al. (2015) ASCO Meeting Abstract 33(15): 8084; Sequist et al. (2015) 372(18): 1700-9; Lee et al. (2014) Cancer Res 74(19Supplement):LB-100; Sakagami et al. (2014) Cancer Res 74(19 Supplement): 1728; Goto et al. (2015) ASCO Meeting Abstract 33(15_Suppl):8014; Jia et al. (2016) Cancer Res 76: 1591-602.

In some embodiments, the targeted drug therapy is an ALK inhibitor (e.g., crizotinib (xalkori), ceritinib (zykadia, LDK378), alectinib (alecensa, R05424802; CH5424802), brigatinib (alunbrig, AP26113), lorlatinib (PF-06463922), TSR-011, RXDX-101 (NMS-E628), X-396, CEP-37440). See, e.g., Tartarone et al. (2017) Med. Oncol. 34(6): 110; Galkin et al. (2007) PNAS 104(1): 270-275; Friboulet et al. (2014) Cancer Discov. 4(6); 662-73; Chen et al. (2013) 56(14): 5673-5674; Shaw et al. (2014) N. Engl. J. Med. 370(13): 1189-1197; Sakamoto et al. (2011) Cancer Cell 19(5): 679-690; Squillace et al. (2013) Cancer Res. 73(8_suppl_: 5655; Mori et al. (2014) 13(2): 329-340; Patnaik et al. (2013) J. Clin. Oncol. 31 (15 suppl); Weiss et al. (2013) J Thorac Oncol. 8(suppl2): S618; Ardini et al. (2009) Mol. Cancer Ther. 8(12suppl): A244; Horn et al. (2014) J. Clin. Oncol. 32(15suppl); Cheng et al. (2012) Mol. Cancer Ther. 11(3): 670-679; Zhang et al. (2011) Cancer Res. 70(8suppl): LB-298; Awad and Shaw (2014) Clin. Adv. Hematol. Oncol. 12(7): 429-439.

In some embodiments, the targeted drug therapy is a heat shock protein 90 inhibitor (e.g, AUY922, ganetspib, AT13387). See, e.g., Pillai et al. (2014) Curr Opin Oncol. 26(2): 159-164; Normant et al. (2011) Oncogene 30(22): 2581-2586; Sequist et al. (2010) J. Clin. Oncol. 28(33): 4953-4960; Sang et al. (2013) Cancer Discov. 3(4): 430-443; Felip et al. (2012) Ann Oncol 23(suppl9); Miyajima et al. (2013) Cancer Res. 73(23): 7022-7033.

In some embodiments, the targeted drug therapy is a RET inhibitor (e.g., cabozantinib (XL184), vandetanib, alectinib, sorafenib, sunitinib, ponatinib) See, e.g., Drilon et al. (2013) Cancer Discov 3:6305; Gautschi et al. (2013) J. Thorac Oncol 8: e43-4; Kodama et al. (2014) Mol. Cancer Ther. 13: 2910-8; Lin et al. (2016) J. Thoracic Oncol. 11(11): 2027-2032; Rosell and Karachaliou (2016) Lancet 17(12): 1623-1625; Falchook et al. (2016) J. Clin Oncol. 34(15): e141-144; Shaw et al. (2013) Nat Rev Cancer 13: 772-787; Gozgit et al. (2013) Cancer Res 73 (Suppl. 1): 2084.

In some embodiments, the targeted drug therapy is a BRAF inhibitor (e.g., dabrafenib, vemurafenib). See, e.g., Planchard et al. (2013) J. Clin. Oncol. 31:8009; Gautschi et al. (2013) Lung Cancer 82: 365-367; Schmid et al. (2015) Lung Cancer 87: 85-87.

In some embodiments, the targeted drug therapy is a MET inhibitor (e.g., onartuzumab, ficlatuzumab, rilotumumab, tivantinib, crizotinib). See, e.g., Spigel et al. (2014) J. Clin. Oncol. 32: 8000; Patnail et al. (2014) Br. J. Cancer 111: 272-280; Gordon et al. (2010): Clin. Cancer Res. 16: 699-710; Sequist et al. (2011) J. Clin. Oncol. 29: 3307-3315; Zou et al. (2007) Cancer Res. 67: 4408-4417; Ou et al. (2011) J. Thorac. Oncol. 6: 942-946.

In some embodiments, the therapeutic intervention is administration of an immunotherapy. See, e.g., Smasundaram and Burns (2017) J. Hematol. Oncol. 10:87. In some embodiments, the immunotherapy is an anti-PD-1 agent (e.g., nivolumab) (Brahmer et al. (2012) N. Engl. J. Med. 366(26): 2455-2465; Gettinger et al. (2016) J. Clin. Oncol. 34(25)), pembrolizumab (Keytruda) (Garon et al. (2015) N. Engl. J. Med. 372(21): 2018-2028), durvalumab), nivolumab (opdivo)). In some embodiments, the immunotherapy is an anti-PD-L1 agent (e.g., atezolizumab (Fehrenbacher et al. (2016) Lancet 387(10030): 1837-1846, Rittmeyer et al. (2017) Lancet 389(10066): 255-265); atezolizumab (Tecentriq)). In some embodiments, the immunotherapy is an anti-CTLA-4 agent (e.g., ipilimumab or tremlimumab). In some embodiments, the immunotherapy is a combination therapy of an anti-PD-1 agent and an anti-CTLA-4 agent (e.g., nivolumab and ipilimumab (Herbset et al. (2015) 21(7): 1514-1524), pembrolizumab and ipilimumab (Gubens et al. (2016) ASCO Meeting Abstracts 34(15_suppl):9027), durvalumab and tremlimumab (NCT02542293. Study of 1st line therapy study of MEDI4736 with tremelimumab versus SoC in non-small-cell lung cancer (NSCLC) (NEPTUNE)).

In some embodiments, the immunotherapy is given in combination with a chemotherapeutic agent (e.g., Rizvi et al. (2016) J. Clin. Oncol. 34(25): 2969-79; Hall et al. (2016) ASCO Meeting Abstracts. 34(15_suppl):TPS9104).

In some embodiments, the therapeutic intervention is chemotherapy (e.g., cisplatin, carboplatin, paclitaxel, albumin-bound paclitaxel, docetaxel, gemcitabine, vinorelbine, irinotecan, etoposide, vinblastine or pemetrexed (alimta)). In some embodiments, the therapeutic intervention is a combination of at least two chemotherapeutic agents.

In some embodiments, the therapeutic intervention is surgery (e.g., a wedge resection (i.e. removal of a small section of diseased lung and a margin of healthy tissue); a segmental resection (segmentectomy) (i.e. removal of a larger portion of lung, but not an entire lobe); a lobectomy (i.e. removal of an entire lobe of one lung); a pneumonectomy (i.e. removal of an entire lung)), or a sleeve resection. The extent of surgical removal will depend on the stage of lung cancer and overall prognosis. In some embodiments, surgery is carried out by video-assisted thoracic surgery (VATS). In some embodiments, the therapeutic intervention is radiofrequency ablation (RFA).

In some embodiments, the therapeutic intervention is radiation therapy. In some embodiments, the radiation therapy is external beam radiation therapy (e.g., three-dimensional conformal radiation therapy (3D-CRT), intensity modulated radiation therapy (IMRT), stereotactic body radiation therapy (SBRT), brachytherapy or a use of radioactive phosphorus.

In some embodiments, the therapeutic intervention further comprises palliative care. In some embodiments, palliative care includes removal of pleural effusion by thoracentesis, pleurodesis or catheter placement. In some embodiments, palliative care includes removal of pericardial effusion by pericardiocentesis, a pericardial window. In some embodiments, the therapeutic intervention is photodynamic therapy (PDT), laser therapy or stent placement.

### Breast Cancer

In some embodiments of any of the methods described herein, the subject may have hereditary breast cancer (Peters et al. (2017) Gynecol Oncol pii: S0090-8258(17)30794-1). In some embodiments, the subject may have triple negative breast cancer (estrogen receptor negative, progesterone receptor negative and HE2-negative), hormone receptor positive (estrogen and/or progesterone receptor positive) breast cancer, hormone receptor negative (estrogen and/or progesterone receptor negative) breast cancer, HER2 positive breast cancer, HER2 negative breast cancer, inflammatory breast cancer or metastatic breast cancer.

In some embodiments wherein a breast cancer cell has been detected in the subject, the subject has at least one mutation in a gene selected from the group consisting of: BRCA1, BRCA2, ATM, CHD1, CHEK2, PALB2, STK11, TP53, HER2 (ERBB2), CDK4/6, AKT1, GATA binding protein 3 (GATA3), RB1, lysine methyltransferase 2C (MLL3), mitogen-activated protein kinase 1 (MAP3K1), CDKN1B, T-box3(TBX3), runt related transcription factor 1 (RUNX1), core binding factor beta (CBFB), phosphoinositide-3-kinase regulatory subunit 1 (PIK3R1), protein tyrosine phosphatase non-receptor type 22 (PTPN22), protein tyrosine phosphatase receptor type D (PTPRD), NF1, splicing factor 3b subunit 1 (SF3B1), cyclin D3 (CCND3), T-box 5 (TBX5), CCCTC-binding factor (CTCF), forkhead box A1 (FOXA1), PI3KCA, PTEN, mitogen-activated protein kinase 4 (MAP2K4), and combinations thereof. See, e.g., Nik-Zainal et al. (2016) Nature 534: 47-54; Bergamaschi et al. (2008) J. Pathol. 214: 357-367; Pleasance et al. (2010) Nature 463: 191-196; The Cancer Genome Atlas Network (2012) Nature 490:61-70; Usary et al. (2004) Oncogene 23: 7669-7678; Bachman et al. (2004) Cancer Biol. Ther. 3: 772-775; Saal et al. (2008) Nature Genet 40: 102-107; Troester et al. (2006) BMC Cancer 6: 276; Chandriani et al. (2009) PLoS One 4: e6693; Matsuda et al. (2017) Breast Cancer Res Treat 163(2): 263-272.

In some embodiments, the targeted drug therapy is a HER2 inhibitor (e.g., trastuzumab (Herceptin), pertuzumab (perjeta); ado-trastuzumab emtansine (T-DM1; Kadcyla); lapatinib (Tykerb), neratinib). See, e.g., Baselga et al. (2012) N Engl J Med 366: 109-119; Konecny et al. (2006) Cancer Res 66: 1630-1639, Xia et al. (2007) Cancer Res. 67: 1170-1175; Gomez et al. (2008) J Clin Oncol 26: 2999-30005; Wong et al. (2009) Clin. Cancer Res. 15: 2552-2558; Agus et al. (2002) Cancer Cell 2: 127-137; Lewis Philips et al. (2008) Cancer Res 68: 9280-9290.

In some embodiments, the targeted drug therapy is a cyclin-dependent kinase inhibitor (e.g., a CDK4/6 inhibitor (e.g., palbociclib (Ibrance®), ribociclin(Kisqali®), abemaciclib) (Turner et al. (2015) N Engl J Med 373: 209-219; Finn et al. (2016) N Eng J Med 375: 1925-1936; Ehab and Elbaz (2016) Breast Cancer 8: 83-91; Xu et al. (2017) J Hematol. Oncol. 10(1): 97; Corona et al. (2017) Cri Rev Oncol Hematol 112: 208-214; Barroso-Sousa et al. (2016) Breast Care 11(3): 167-173)).

In some embodiments, the targeted drug therapy is a PARP inhibitor (e.g., olaparib (AZD2281), veliparib (ABT-888), niraparib (MK-4827), talazoparib (BMN-673), rucaparib (AG-14699), CEP-9722) See, e.g., Audeh et al. (2010) Lancet 376: 245-251; Fong et al. (2009) N Engl J Med 361: 123-134; Livrahi and Garber (2015) BMC Medicine 13: 188; Kaufamn et al. (2015) J Clin. Oncol. 33: 244-250; Gelmon et al. (2011) Lancet Oncol. 12: 852-61; Isakoff et al. (2011) Cancer Res 71:P3-16-05; Sandhu et al. (2013) Lancet Oncol 14:882-92; Tutt et al. (2010) Lancet 376: 235-44; Somlo et al. (2013) J. Clin. Oncol. 31: 1024; Shen et al. (2013) CLin. Cancer Res. 19(18): 5003-15; Awada et al. (2016) Anticancer Drugs 27(4): 342-8.

In some embodiments, the targeted drug therapy is a mTOR inhibitor (e.g., everolimus (afinitor)). See, e.g., Gong et al. (2017) Oncotarget doi: 10.18632/oncotarget.16336; Louseberg et al. (2017) Breast Cancer 10: 239-252; Hare and Harvey (2017) Am J Cancer Res 7(3): 383-404.

In some embodiments, the targeted drug therapy is a heat shock protein 90 inhibitor (e.g., tanespimycin) (Modi et al. (2008) J. Clin Oncol. 26: s1027; Miller et al. (2007) J. Clin. Oncol. 25:s1115; Schulz et al. (2012) J Exp Med 209(2): 275-89).

In some embodiments, the targeted drug therapy further includes a bone-modifying drug (e.g., a bisphosphonate or denosumab (Xgeva)). See, e.g., Ethier et al. (2017) Curr Oncol Rep 19(3): 15; Abdel-Rahman (2016) Expert Rev Anticancer Ther 16(8): 885-91.

In some embodiments, the therapeutic intervention is a hormone (e.g., a luteinizing-hormone-releasing hormone (LHRH) agonist). In some embodiments, the LHRH agonist is goserelin (Zoladex®) or leuprolide (Lupron®). In some embodiments, the therapeutic intervention is an anti-estrogen compound (e.g., tamoxifen, fulvestrant (faslodex)). In some embodiments, the therapeutic intervention is an aromatase inhibitor (e.g., letrozole (Femara®), anastrozole (Arimidex®) or exemestane (Aromasin®).

In some embodiments, the therapeutic intervention is surgery (e.g., a lumpectomy, a single mastectomy, a double mastectomy, a total mastectomy, a modified radical mastectomy, a sentinel lymph node biopsy, an axillary lymph node dissection; breast-conserving surgery). The extent of surgical removal will depend on the stage of breast cancer and overall prognosis.

In some embodiments, the therapeutic intervention is radiation therapy. In some embodiments, the radiation therapy is partial breast irradiation or intensity-modulated radiation therapy.

In some embodiments, the therapeutic intervention is chemotherapy (e.g., capecitabine (xeloda), carboplatin (paraplatin), cisplatin (platinol), cyclophosphamide (neosar), docetaxel (docefrez, taxotere), doxorubicin (Adriamycin), pegylated liposomal doxorubicin (doxil), epirubicin (ellence), fluorouracil (5-FU, adrucil), gemcitabine (gemzar), methotrexate, paclitaxel (taxol), protein-bound paclitaxel (abraxane), vinorelbine (navelbine), eribulin (halaven), or ixabepilone (ixempra)). In some embodiments, the therapeutic intervention is a combination of at least two chemotherapeutic agents (e.g., doxorubicin and cyclophosphamide (AC); epirubicin and cyclophosphamide (EC); cyclophosphamide, doxorubicin and 5-FU (CAF); cyclophosphamide, epirubicin and 5-FU (CEF); cyclophosphamide, methotrexate and 5-FU (CMF); epirubicin and cyclophosphamide (EC); docetaxel, doxorubicin and cyclophosphamide (TAC); docetaxel and cyclophosphamide (TC).

Non-limiting aspects of these methods are described below, and can be used in any combination without limitation. Additional aspects of these methods are known in the art.

### EXAMPLES

The invention is further described in the following examples, which do not limit the scope of the claims.

### Example 1. Circulating Tumor DNA (ctDNA) Dynamics During Immune Checkpoint Blockade

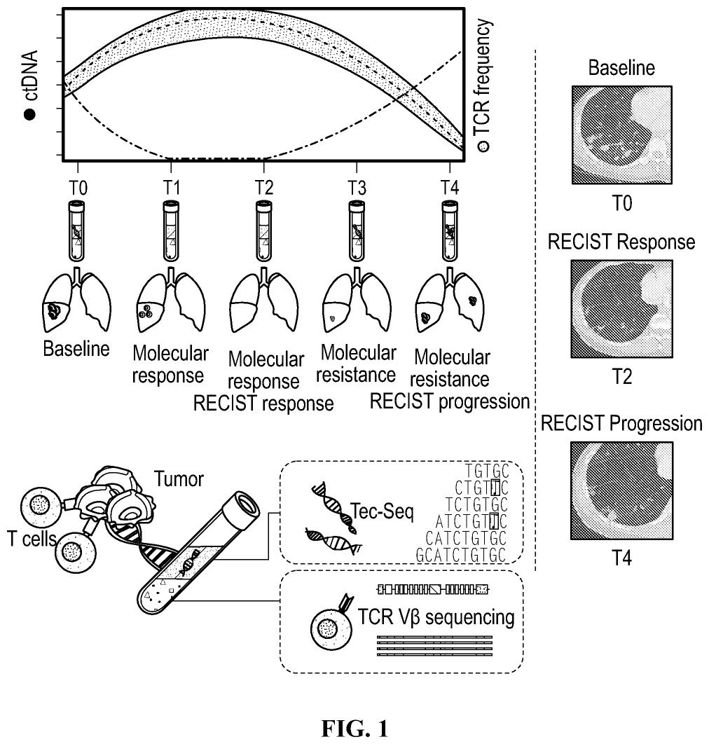

Serial blood samples from 15 patients with metastatic non-small cell lung cancer (NSCLC) were analyzed during immune checkpoint blockade (Table 51). Blood samples were prospectively collected prior to therapy, at an early time point between 4 and 8 weeks from treatment initiation and at additional serial time points during therapy until the time of disease progression (Tables S2 and S3). ctDNA was measured using the TEC-Seq approach (Phallen et al., Science Transl Med, (403), 2017) and the TCR repertoire was studied longitudinally by means of TCR sequencing (FIG. 1, Anagnostou et al., Cancer Discovery, 7(3): 264-276, 2017). Given the possibility of hematopoietic alterations which may be detected in the plasma, especially in heavily treated patients, parallel tumor samples were evaluated from the same patients to distinguish tumor-specific from hematopoietic sequence alterations in cell-free DNA (Tables S4-S9). The median follow-up duration was 12.7 months (range 3.8-37.8 months) and median duration of treatment was 6.6 months (range 1-19.6 months). The response to immune checkpoint blockade was evaluated using standard computed tomographic (CT) imaging. Changes in tumor burden were assessed by RECIST 1.1 (31).

ctDNA was detected in 13 of 15 patients either at baseline (n=12) or at other time points when baseline samples were not available (n=1), with a median mutant allele fraction of 1.08% (range 0.09%-20.4%). For patients with detectable ctDNA, an average of two tumor-specific alterations were detected (median 1, range 1-3) in eight driver genes, including those commonly altered in lung cancer (Table 1, Tables S5-S7).

TABLE 1
Genes Analyzed by TEC-sequencing

| Gene | Region Analyzed | Gene | Region Analyzed |
|---|---|---|---|
| ABL1 | Specific Exons | IDH1 | Specific Exons |
| AKTI | Specific Exons | IDH2 | Specific Exons |
| ALK | Full Coding Region | JAK2 | Full Coding Region |
| APC | Specific Exons | JAK3 | Specific Exons |
| AR | Full Coding Region | KDR | Specific Exons |
| ATM | Specific Exons | KIT | Full Coding Region |
| BRAF | Full Coding Region | KRAS | Full Coding Region |
| CDH1 | Specific Exons | MAP2K1 | Specific Exons |
| CDK4 | Full Coding Region | MET | Specific Exons |
| CDK6 | Full Coding Region | MLH1 | Specific Exons |
| CDKN2A | Specific Exons | MPL | Specific Exons |
| CSF1R | Specific Exons | MYC | Specific Exons |
| CTNNB1 | Specific Exons | NPM1 | Specific Exons |
| DNMT3A | Specific Exons | NRAS | Full Coding Region |
| EGFR | Full Coding Region | PDGFRA | Full Coding Region |
| ERBB2 | Specific Exons | PIK3CA | Full Coding Region |
| ERBB4 | Full Coding Region | PIK3R1 | Specific Exons |
| ESR1 | Full Coding Region | PTEN | Full Coding Region |
| EZH2 | Specific Exons | PTPN11 | Specific Exons |
| FBXW7 | Specific Exons | RB1 | Specific Exons |
| FGFR1 | Specific Exons | RET | Specific Exons |
| FGFR2 | Specific Exons | SMAD4 | Specific Exons |
| FGFR3 | Specific Exons | SMARCB1 | Specific Exons |
| FLT3 | Specific Exons | SMO | Specific Exons |
| GNA11 | Specific Exons | SRC | Specific Exons |
| GNAQ | Specific Exons | STK11 | Full Coding Region |
| GNAS | Specific Exons | TERT | Specific Exons |
| HNF1A | Specific Exons | TP53 | Full Coding Region |
| HRAS | Full Coding Region | IDH1 | Specific Exons |

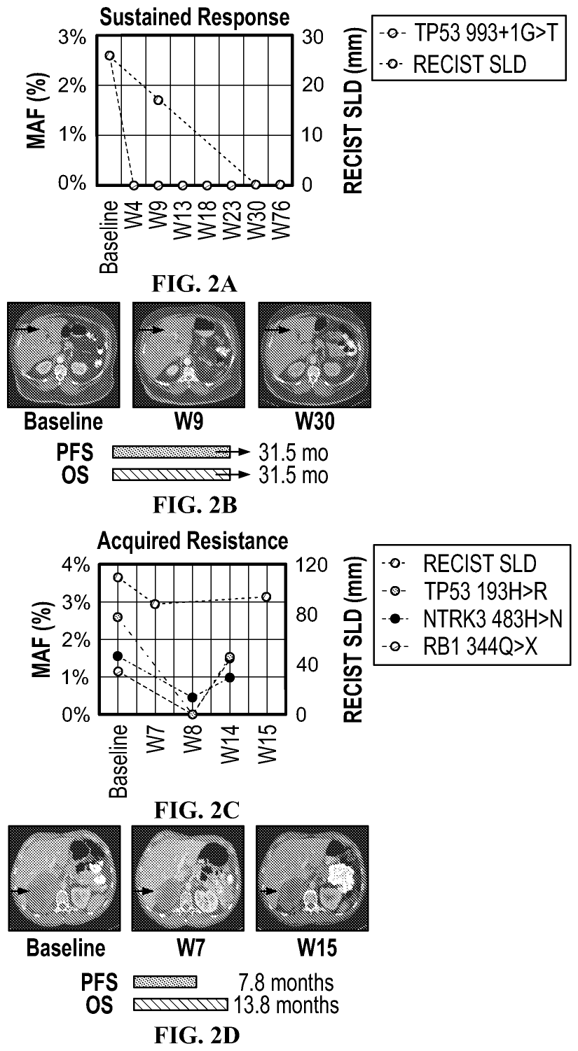

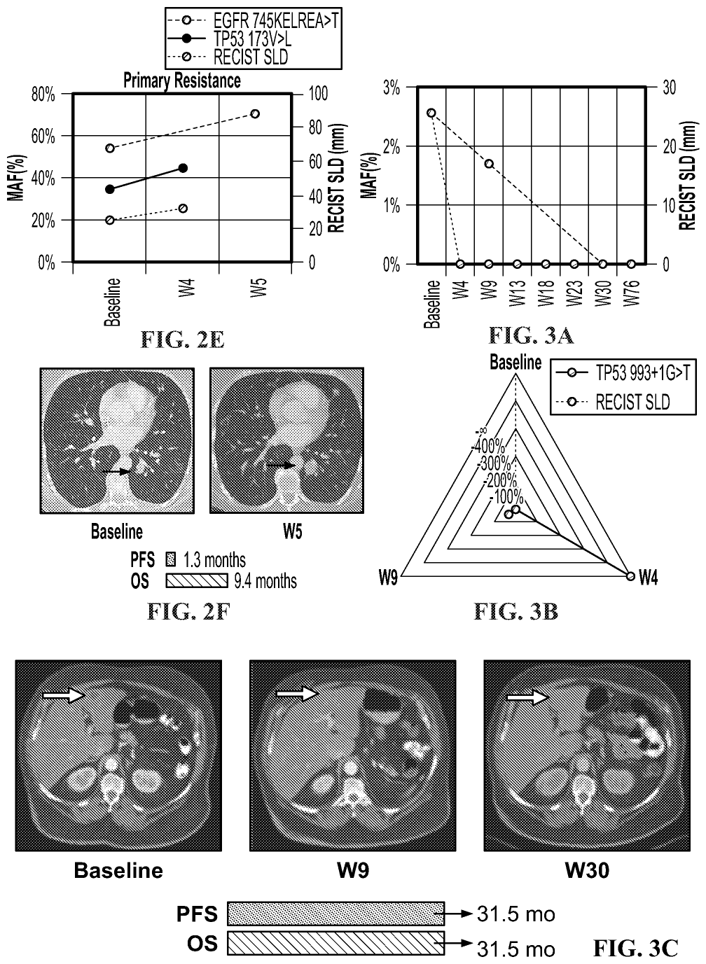

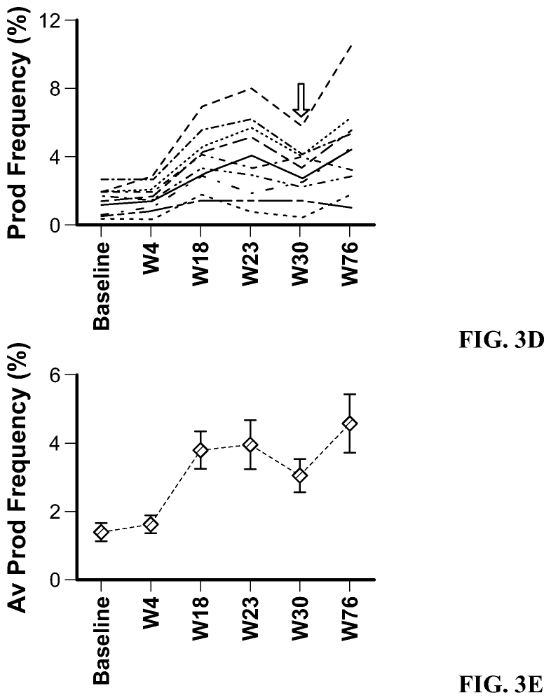

Three patterns of molecular response were detected in ctDNA for patients treated with immune checkpoint inhibitors (FIGS. 2A-F). Among the six patients with a clinical response, individuals had a dramatic reduction in ctDNA to undetectable levels at 4-8 weeks from treatment initiation. As an example, for patient CGLU111 with a sustained clinical response, ctDNA-based molecular analyses showed a complete response at week 4, more than 26 weeks earlier than complete radiographic response determined by RECIST 1.1 (FIGS. 3A-E).

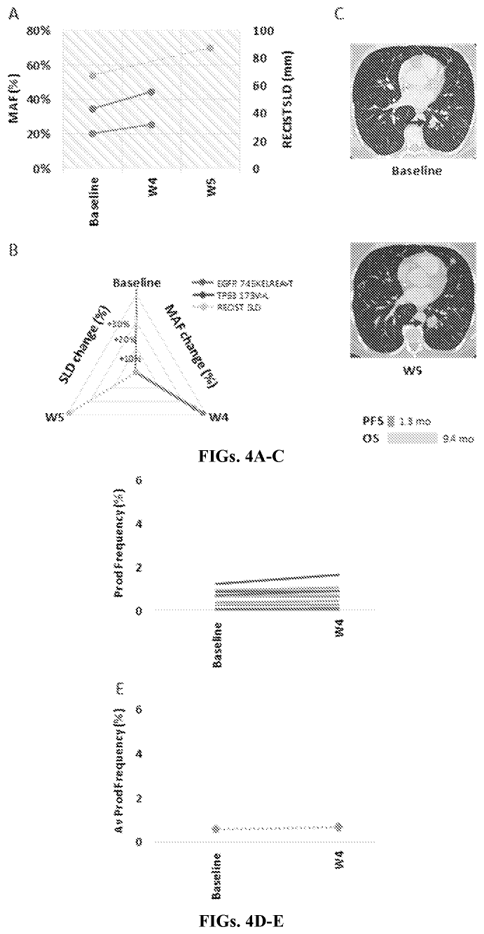

In contrast, for patients with primary resistance to immune checkpoint blockade, ctDNA levels had limited fluctuations or displayed a rise after therapeutic initiation. As a representative patient, ctDNA levels in CGLU121 continued to rise from the time of initiation of immune checkpoint blockade, consistent with radiographic disease progression (FIGS. 4A-E). A total of five patients showed ctDNA features of primary molecular resistance that were subsequently confirmed by radiographic disease progression.

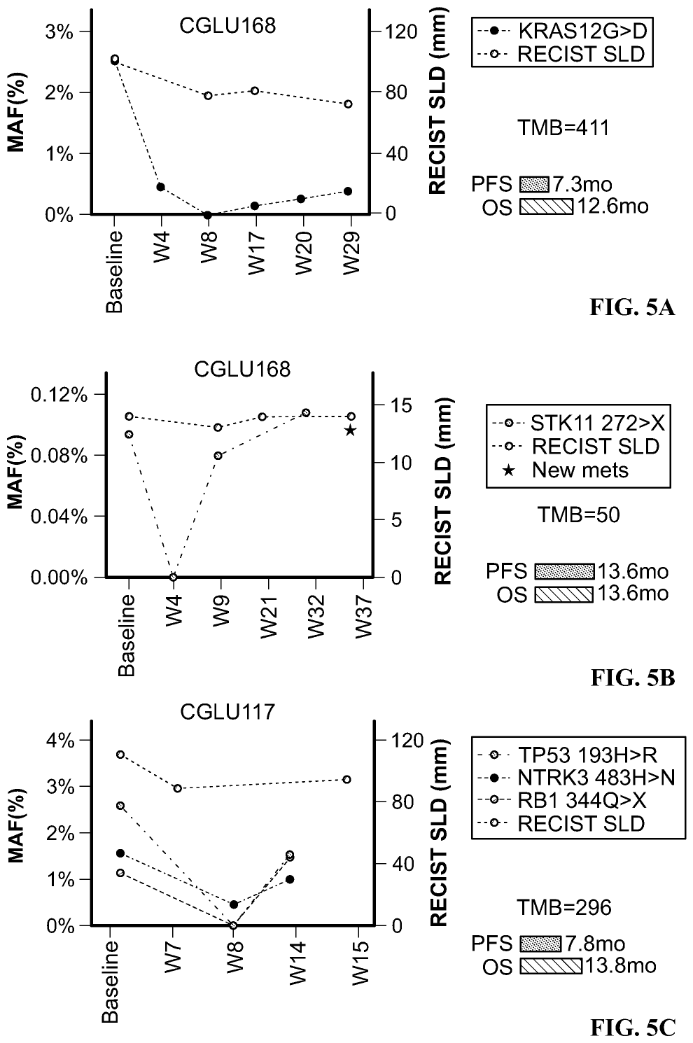

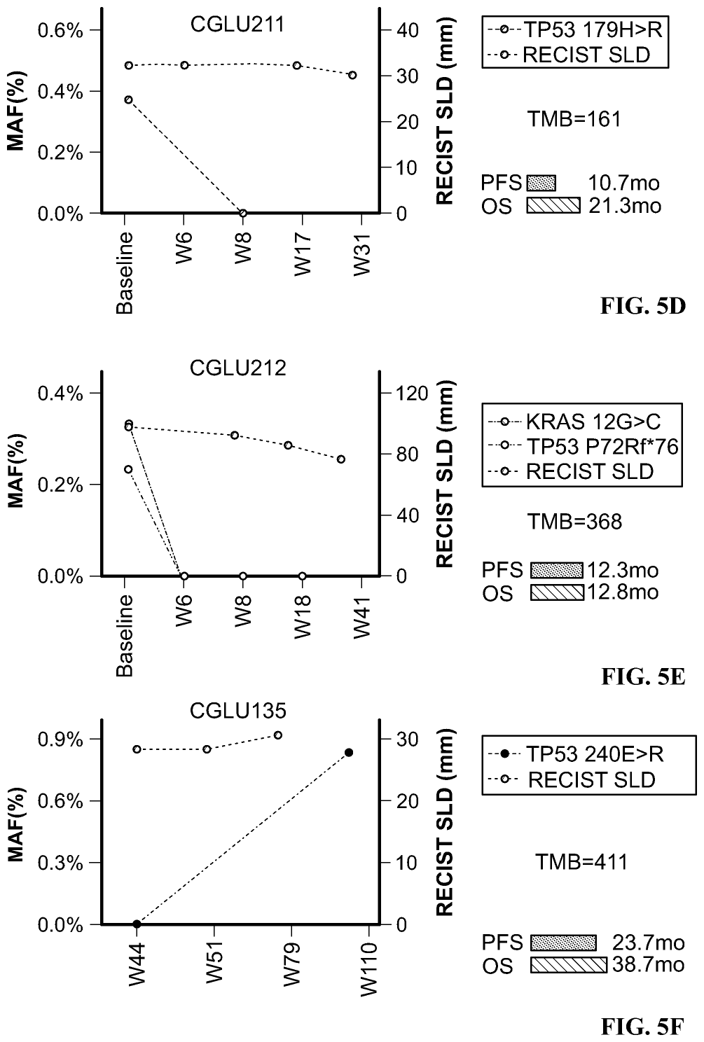

The third observed pattern, seen in four of the clinical responders, was one consistent with molecular acquired resistance, where ctDNA dynamics reflected clonal evolution under selective pressure of anti-PD1 therapy and emergence of immune escape. In such cases, at least one sub-clone represented by tumor-specific variants were undetectable at the time of response followed by increase in mutant allele fraction of tumor-specific mutations at the time of acquired resistance (FIGS. 5A-F).

ctDNA molecular responses were more rapid and accurate than imaging or other predictive biomarkers. For example, four patients with stable disease by radiographic imaging showed a clear molecular response pattern, with ctDNA elimination between week 4 and 8 from immune checkpoint blockade initiation. All four patients derived clinical benefit from PD-1 blockade (PFS and OS ranging from 7.3-13.6 and 12.6-21.3 respectively), suggesting that radiographic imaging failed to detect the magnitude of therapeutic response. Interestingly the tumor mutation burden (TMB) for these cases widely varied from 50 to 411 (Table S9), indicating that the ctDNA-based early molecular response signature can be a more accurate predictor of eventual response to checkpoint blockade compared to TMB.

### Example 2. ctDNA Dynamics Predict Response Earlier than Radiographic Imaging and Predict Long-Term Outcome

In patients with primary resistance, radiographic based tumor progression followed ctDNA-based molecular progression and was detected between week 6 and 16. For the fraction of clinical responders that developed acquired resistance, emergence of molecular resistance preceded disease progression on radiographic imaging by an average of 20 weeks. Overall, ctDNA-based molecular responses were detected on average 5.4 weeks earlier than conventional RECIST1.1 response assessment (5.2 vs 10.6 weeks, p=0.0003).

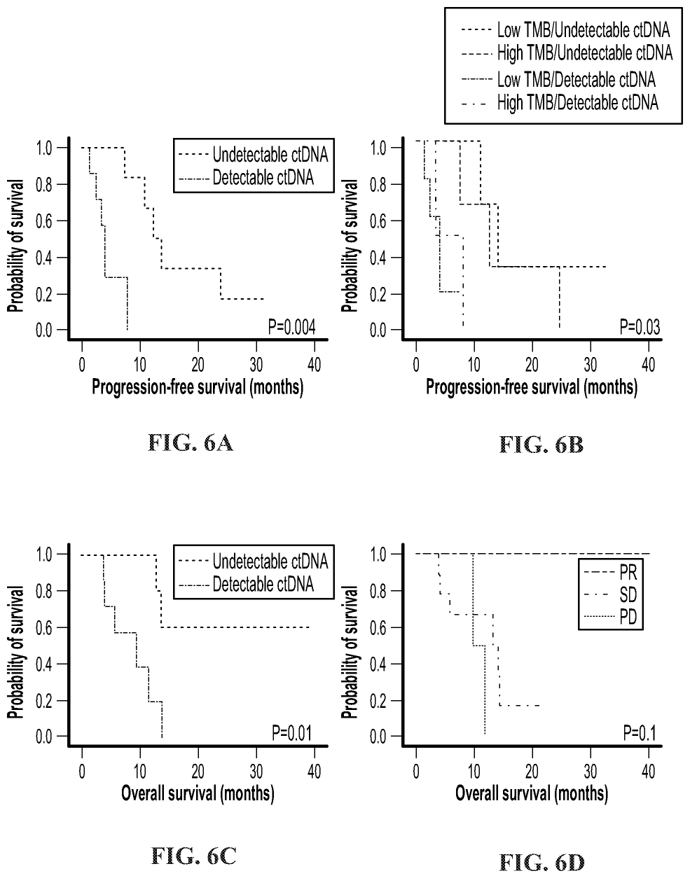

ctDNA clearance at 4 to 8 weeks was a significant prognostic factor for progression-free (PFS) and overall survival (OS). Patients with a reduction of ctDNA to undetectable levels demonstrated a significantly longer PFS and OS compared to patients with no evidence of ctDNA elimination (log rank p=0.004 and p=0.01 respectively, FIGS. 6A and C). Interestingly, radiographic imaging failed to predict therapeutic benefit from anti-PD1 therapy for these patients (FIG. 6D) and TMB alone failed to accurately distinguish responders from non-responders. When TMB and ctDNA were combined the ctDNA-based molecular responders clustered together independent of the TMB (FIG. 6B) for both PFS and OS.

### Example 3. Peripheral TCR Landscape and Therapeutic Outcome

To determine how immune checkpoint blockade affects the peripheral TCR repertoire and whether there are TCR clonotype dynamic changes reflective of a systemic anti-tumor immune response, TCR clones found in the tumor microenvironment were analyzed using TCR sequencing. TCR clonotype dynamics were also investigated in the peripheral blood, identifying TCR clones with a statistically significant expansion from baseline. Nine of the 15 patients had available samples from both tumor infiltrating lymphocytes as well as peripheral blood lymphocytes for analysis (Tables S3 and S10).

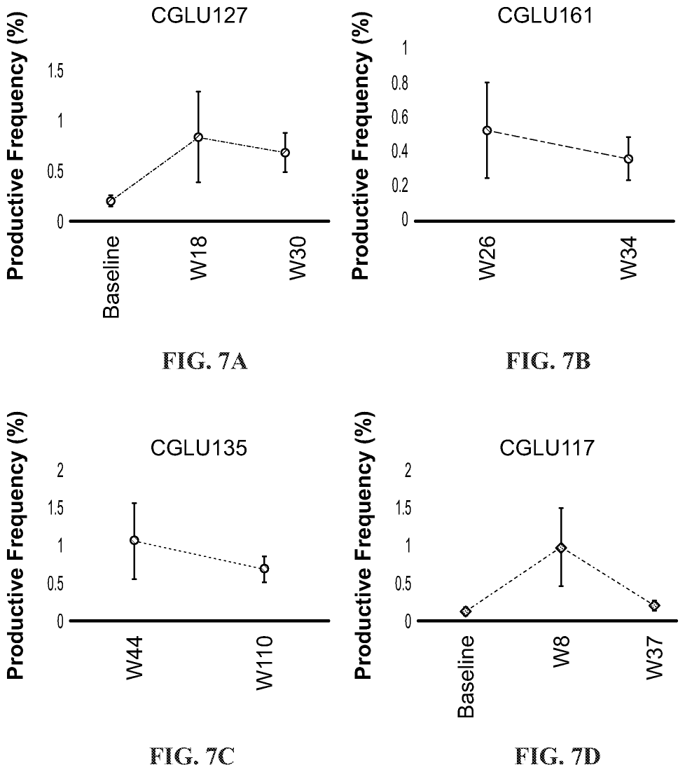

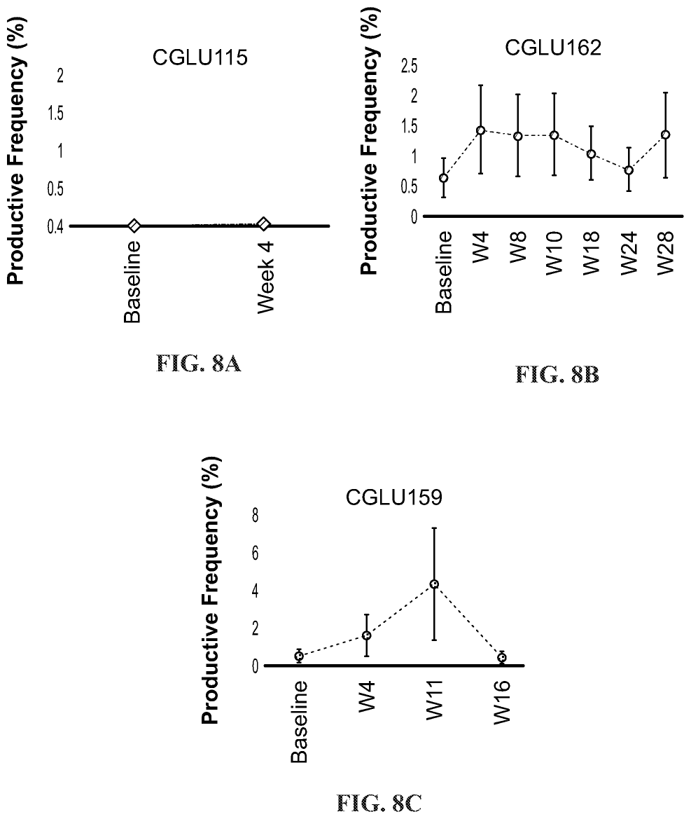

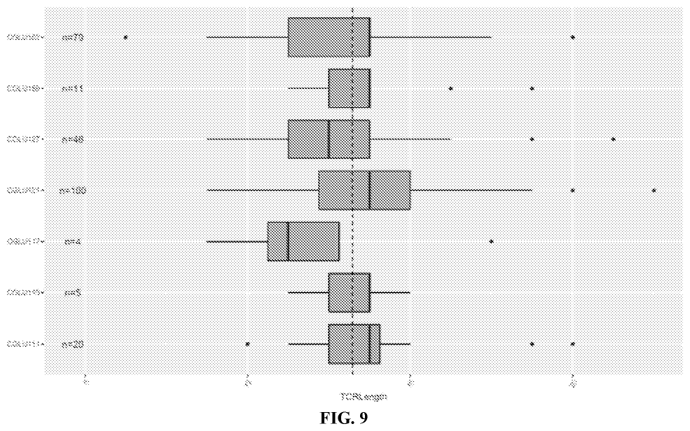

Similar to ctDNA analyses, distinct patterns in TCR clonotype dynamics were discovered among the analyzed patients. For patients with clinical responses to immune checkpoint blockade, a statistically significant oligoclonal expansion of pre-existing intra-tumoral T cell clones was observed in peripheral blood at the time of radiographic response to PD1 blockade (CGLU111, CGLU117 and CGLU127) (FIG. 2, FIGS. 7A-D, Tables S11-S13). For patients that developed acquired resistance (CGLU117, CGLU127, CGLU135 and CGLU161) (FIG. 8), productive frequencies of intratumoral clones significantly decreased in peripheral blood at the time of acquired resistance (FIG. 7, Tables S12-S15), with a timing that was similar to ctDNA analyses for most cases.

In contrast, for patients CGLU121 and CGLU115 that had primary resistance to immunotherapy, no differentially abundant TCR clones were identified among serial peripheral blood samples (FIG. 3 and FIG. 8). These patients progressed radiographically within 5-13 weeks from initiation of therapy and, in line with the clinical course, there was no evidence of TCR clonal expansion among the intratumoral TCR repertoire. A transient oligoclonal TCR expansion was observed for non-responding patient CGLU159 at week 11, however productive frequencies of differentially abundant clones quickly decreased to baseline levels at week 16, which coincided with disease progression (FIG. 8, Table S16). Similarly patient CGLU162 had 10 intratumoral TCR clones with differential abundance at week 10 compared to baseline but was a ctDNA molecular non-responder (FIG. 8), Table S17), suggesting that for these patients, ctDNA kinetics can more accurately predict therapeutic outcome.

No shared TCR clones were identified among the differentially expanded ones for all patients analyzed, which was consistent with the private mutation-associated neoantigen repertoire of these tumors (Table S9). Putative shared CDR3 motifs were evaluated among significantly expanded TCR clones employing the grouping of lymphocyte interactions by paratope hotspots algorithm (15). Interestingly, TCR clones CSARVGVGNTIYF (SEQ ID NO: 1) and CSARSGVGNTIYF (SEQ ID NO: 2), that were differentially abundant at the time of response to immune checkpoint blockade for patient CGLU127 and CGLU135, respectively, clustered together, suggesting a common specificity to a tumor- or mutation-associated antigen. The related CDR3 motifs corresponded to the TCR-peptide contact residues suggesting that these TCRs are likely to recognize the very similar pMHC ligands.

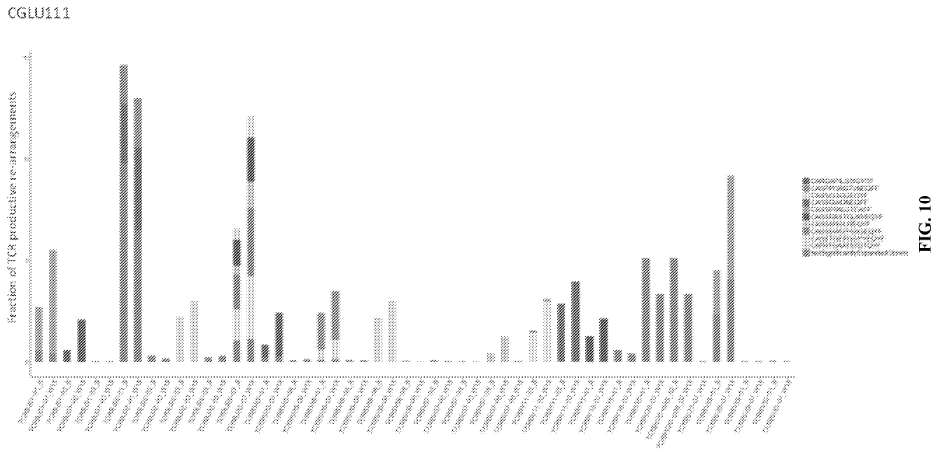

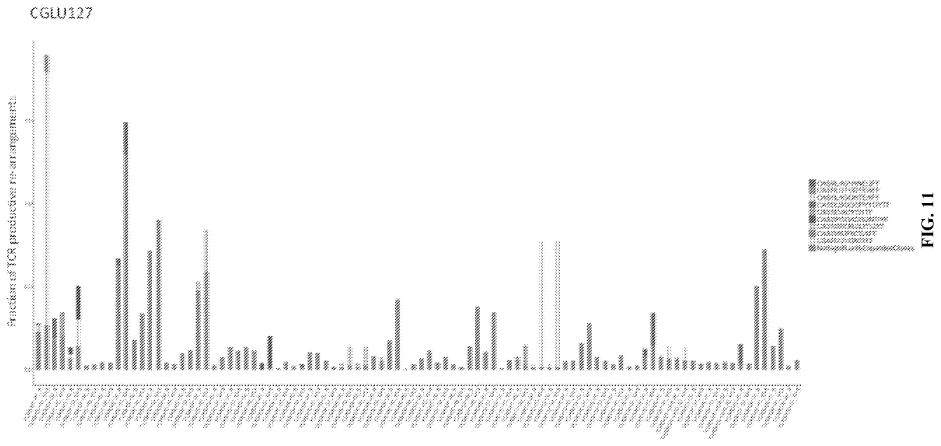

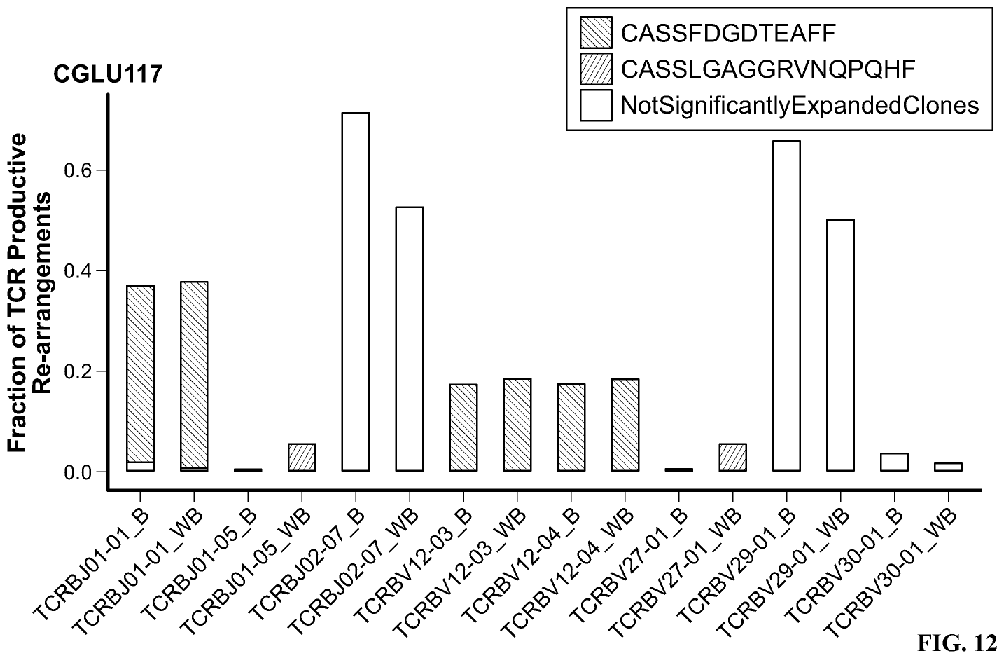

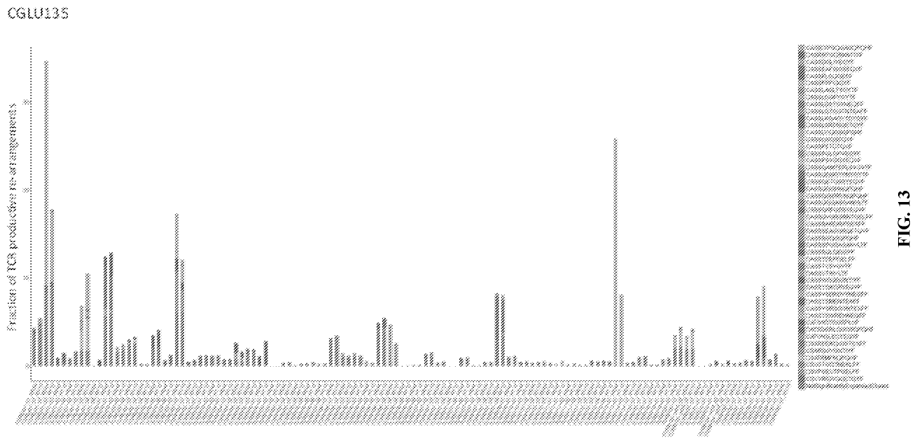

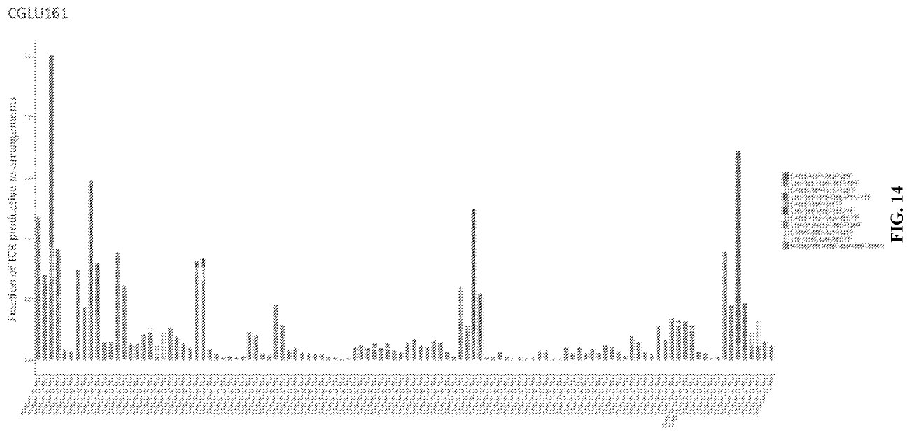

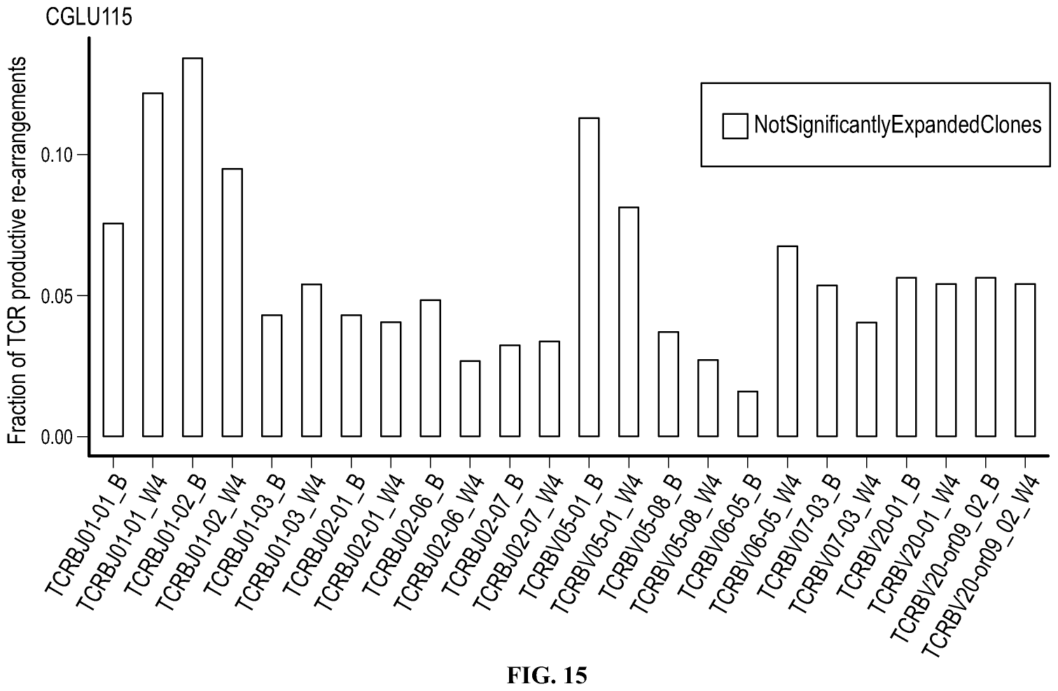

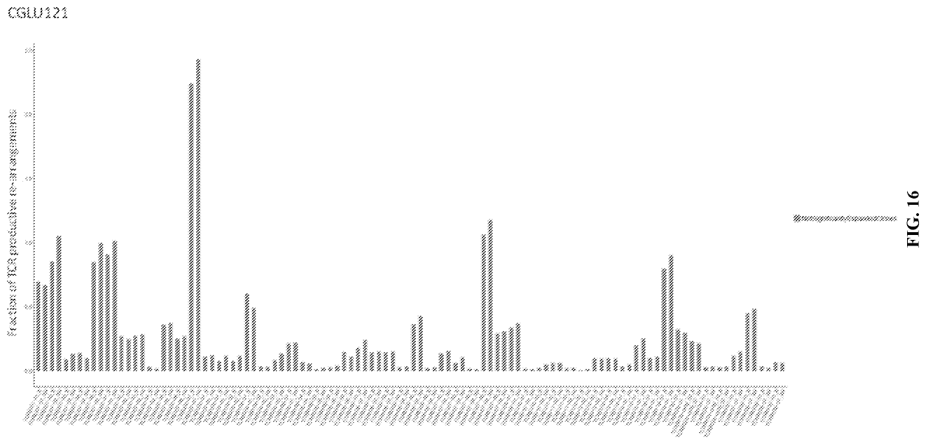

Subsequently potential differential sequence features were investigated focusing on Variable (V) and Joining (J) gene usage and CDR3 lengths among different time points for each patient. No differences in CDR3 lengths were identified among baseline peripheral T cell samples across patients (FIG. 9). Interestingly, usage of specific V and J gene segments increased at the time of response compared to baseline (FIGS. 10-12) and decreased at the time of acquired resistance (FIG. 13 and FIG. 14), consistent with the clonal expansions and contractions observed. In contrast, no dynamic changes in V gene usage were identified for patients with primary resistance to anti-PD1 (FIG. 15 and FIG. 16). These findings on differential V gene usage may suggest clonotypic amplifications of specific immune subsets (CD8+ vs. CD4+) during immune checkpoint blockade (16).

In summary, dynamic assays were developed that capture the tumor-immune system equilibrium and assess immune editing of neoantigens during immunotherapy. These approaches are superior to conventional radiographic response assessment and may be preferable to analyses of static time points such as TMB obtained at baseline. In addition to more accurately predicting long-term response to immunotherapy, therapeutic outcome was predicted on average 5.4 weeks earlier than radiographic imaging. This will help guide early therapeutic decisions to ensure that an ineffective treatment is discontinued as well as allow response adaptive combination and sequencing of subsequent therapies. A dynamic biomarker-driven approach can inform choice of monotherapy versus combination immune-chemotherapy or can be used to devise a step-up approach, where patients with molecular resistance are identified early on and treated with an intensified immune-chemotherapy schema.

### Example 4. Methods

### Patient Characteristics

The study group consisted of 15 metastatic NSCLC patients treated with immune checkpoint blockade as a standard of care (n=11) or in the setting of a clinical trial (n=4) between October 2014 and August 2016. The studies were conducted in accordance with the Declaration of Helsinki, were approved by the Institutional Review Board (IRB) and patients provided written informed consent for sample acquisition for research purposes. Clinical characteristics for all patients are summarized in Table S1.

### Treatment and Assessment of Therapeutic Response

Therapeutic responses were evaluated by the Response Evaluation criteria in Solid Tumors (RECIST) version 1.1 (31). Baseline disease burden was determined by the sum of the longest diameters of target lesions as determined by RECIST 1.1 criteria. After baseline imaging, radiographic evaluation was performed at 5-10 week intervals or as clinically indicated; of the 15 patients analyzed, 1 achieved complete response (CGLU111), 3 patients achieved partial response (CGLU127, CGLU135 and CGLU161) and 9 achieved SD (CGLU115, CGLU117, CGLU159, CGLU160, CGLU162, CGLU168, CGLU203, CGLU211, and CGLU212) as best overall response. For two patients (CGLU121 and CGLU243) CT imaging revealed progressive disease at first assessment. Of the 3 patients with partial response, all eventually developed acquired resistance. PFS and OS were defined as the time elapsed between the date of treatment initiation and the date of disease progression or death from disease, or the date of death, respectively (Table S1).

### Tumor Tissue and Blood Sample Characteristics

For all patients, at least 2 serial blood samples (median 4, range 2-8) were collected over the course of treatment for isolation of plasma and extraction of cell-free DNA for genomic analyses. A total of 51 serial plasma samples were analyzed that were obtained prior to anti-PD1, at 4-8 weeks and additional time points during therapy for all patients except for CGLU135 and CGLU161. For these two patients, baseline blood was not available and blood samples from the time of radiographic response and the time of acquired resistance were analyzed. A detailed description of the time points analyzed is shown in Table S2. Baseline tumors were analyzed by whole exome sequencing for each patient, with the exception of CGLU168 for which a tumor specimen from the time of resistance to immune checkpoint blockade was used (Table S4). All tumor samples were provided as formalin fixed, paraffin embedded blocks (FFPE).

Sample Preparation and Next-Generation Sequencing of cfDNA

Whole blood was collected in K2 EDTA tubes; plasma and cellular components were separated by centrifugation at 800 g for 10 minutes at 4° C. Plasma was centrifuged a second time at 18,000 g at room temperature to remove any remaining cellular debris and stored at −80° C. until the time of DNA extraction. DNA was isolated from plasma using the QiAmp® Circulating Nucleic Acids Kit (Qiagen GmbH, Hilden DE). TEC-Seq next-generation sequencing cell-free DNA libraries were prepared from 12 to 125 ng of cfDNA. Genomic libraries were prepared as previously described and targeted capture was performed using the Agilent SureSelect reagents and a custom set of hybridization probes targeting 58 genes, described in Supplementary Table S7 (8). TEC-Seq libraries were sequenced using 100 bp paired end runs on the Illumina HiSeq® 2500 (Illumina, San Diego, Calif.). The analytical performance and validation including sensitivity and specificity and limits of detection of our ctDNA platform have been recently reported (8).

Genomic alterations in ctDNA were cross-referenced against each patient's tumor-specific genomic alterations identified by whole exome sequencing of the matched tumors to identify bona fide tumor specific ctDNA variants. Variants identified in ctDNA as previously described (8) as well as in the matching tumor with a MAF of >2% were considered tumor-specific.

Primary Processing of cfDNA Next-Generation Sequencing Data and Identification of Putative Somatic Mutations

Primary processing of next-generation sequence data for cfDNA samples was performed as previously described (8) using Illumina CASAVA software (v1.8), including demultiplexing and masking of dual index adapter sequences. Sequence reads were aligned against the human reference genome (hg19) using Novoalign with additional realignment of select regions using the Needleman-Wunsch method (32). Next, candidate somatic mutations, consisting of point mutations, small insertions, and deletions were identified using VariantDx™ (32) across the targeted regions of interest. VariantDx™ examined sequence alignments of cfDNA plasma samples while applying filters to exclude alignment and sequencing artifacts as previously described (8). Specifically, an alignment filter was applied to exclude quality failed reads, unpaired reads, and poorly mapped reads in the plasma. A base quality filter was applied to limit inclusion of bases with reported Phred quality score>30. Criteria for calling alterations in cfDNA have been previously described (8). TEC-Seq characteristics are shown in Tables S5 and S6.

Definition of Tumor-Derived cfDNA

Genomic alterations in ctDNA were cross-referenced against each patient's tumor-specific genomic alterations identified by whole exome sequencing of the matched tumors (Table S9) to identify bona fide tumor specific ctDNA variants. Variants identified in ctDNA as previously described (8) as well as in the matching tumor with a MAF of >2% were considered tumor-specific.

### Whole-Exome Sequencing and Identification of Somatic Mutations

Whole exome sequencing was performed on pre-treatment tumor and matched normal samples (Table S8). Tumor samples underwent pathological review for confirmation of lung cancer diagnosis and assessment of tumor purity. Slides from each FFPE block were macrodissected to remove contaminating normal tissue. Matched normal samples were provided as peripheral blood. DNA was extracted from patients' tumors and matched peripheral blood using the QiAmp® DNA FFPE and QiAmp® DNA blood mini kit respectively (Qiagen, Calif.). Fragmented genomic DNA from tumor and normal samples was used for Illumina TruSeq library construction (Illumina, San Diego, Calif.) and exonic regions were captured in solution using the Agilent SureSelect v.4 kit (Agilent, Santa Clara, Calif.) as previously described (32-34). Paired-end sequencing, resulting in 100 bases from each end of the fragments for the exome libraries was performed using Illumina HiSeq® 2000/2500 instrumentation (Illumina, San Diego, Calif.). The mean depth of coverage for the tumors was 214x, allowing us to identify sequence alterations and copy number changes in >20,000 genes (Table S9).

Somatic mutations were identified using the VariantDx™ custom software for identifying mutations in matched tumor and normal samples (32). Prior to mutation calling, primary processing of sequence data for both tumor and normal samples were performed using Illumina CASAVA software (version 1.8), including masking of adapter sequences. Sequence reads were aligned against the human reference genome (version hg19) using ELAND with additional realignment of select regions using the Needleman-Wunsch method (35). Candidate somatic mutations, consisting of point mutations, insertions, deletions as well as copy number changes were then identified using VariantDx™ across the whole exome as previously described (14).

### T Cell Receptor Sequencing and Differential Expansion Analyses

TCR clones were evaluated in pre-treatment tumor tissue (with the exception of CGLU117, where tumor tissue from the time of resistance was also analyzed), and 31 serial peripheral blood lymphocytes (PBLs) by next generation sequencing (Table S10). DNA from pre-treatment tumor samples and PBLs was isolated by using the QiAmp® DNA FFPE and QiAmp® DNA blood mini kit respectively (Qiagen, Calif.). TCR-β CDR3 regions were amplified using the survey (tumor) or deep (PBLs) ImmunoSeq assay in a multiplex PCR method using 45 forward primers specific to TCR Vβ gene segments and 13 reverse primers specific to TCR Jβ gene segments (Adaptive Biotechnologies) (36, 37). Productive TCR sequences were further analyzed. TCR sequencing data from TILs was used to identify tumor-specific TCR clonotypes in the peripheral blood. Peripheral TCR clones achieving a frequency of at least 0.005% were evaluated for differential abundance between baseline and the time of radiographic response using Fisher's exact test with False Discovery Rate (FDR) p-value correction (corrected P≤0.05). Those differentially abundant clones also found in the tumor were further selected to determine their frequencies in peripheral blood prior to treatment, at the time of response and upon emergence of resistance (Tables S11-S17). To cluster significantly expanded intratumoral TCR-f3 CDR3s based on potential recognition specificity, we employed the GLIPH method (Grouping of Lymphocyte Interactions by Paratope Hotspots) (15).

### CDR3 and VJ Gene Usage Analyses

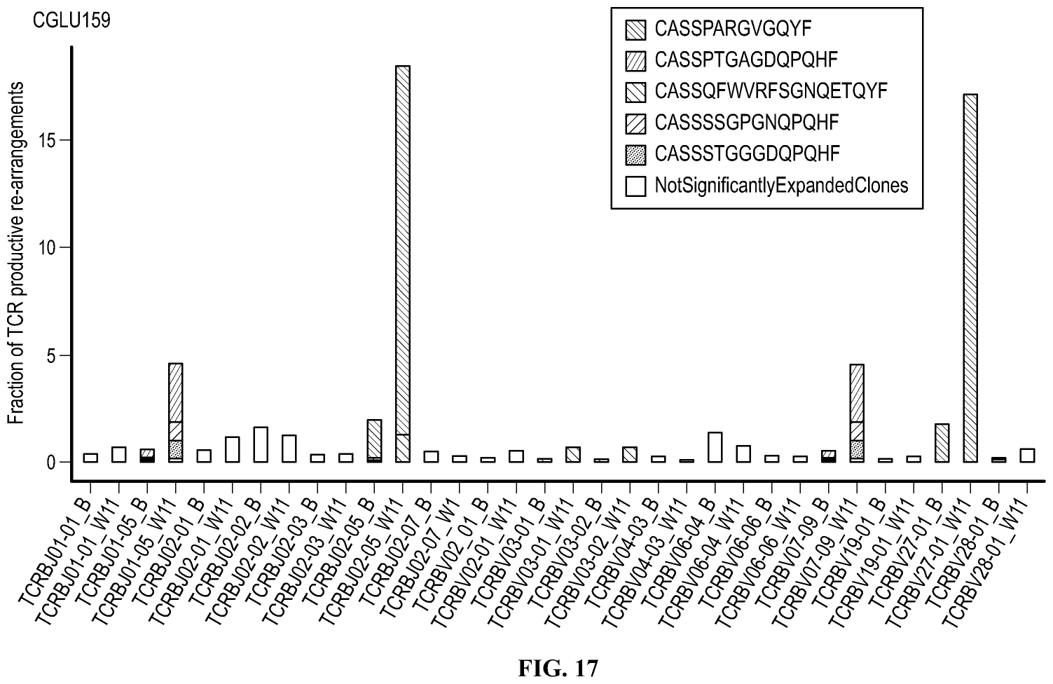

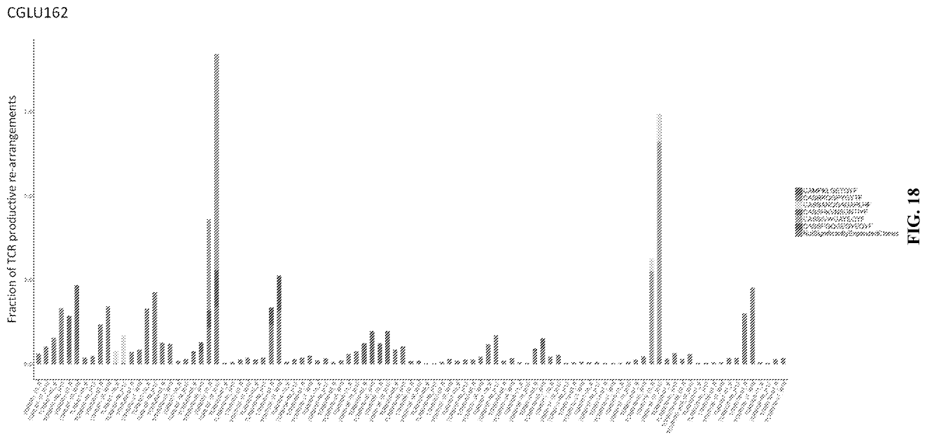

Subsequent to initial filtering, we noted that a large proportion of non-significant clones still had low frequencies at nearly all their time points. A further refinement step to further reduce noise in the data was undertaken to eliminate clones that did not have frequencies beyond a mean rate of 5 counts. Thus, when 2 points were examined, the total sum of counts were greater or equal to 10. Using these data, we examined the usage of CDR3b Variable (V) and Joining (J) regions, and their overall clonal composition by known significant clones at the 2 time points (FIGS. 10-18). CDR3b length was analyzed at baseline and respective distributions are shown in FIG. 9.

### Statistical Analyses

ctDNA values were dichotomized as detectable and undetectable. Characteristics for each group were compared using chi-square or Fischer's exact test for categorical variables. The median point estimate and 95% CI for PFS and OS were estimated by the Kaplan-Meier method. Survival curves were compared by using the log-rank test.

### REFERENCES

1. L. Horn et al., Nivolumab Versus Docetaxel in Previously Treated Patients With Advanced Non-Small-Cell Lung Cancer: Two-Year Outcomes From Two Randomized, Open-Label, Phase III Trials (CheckMate 017 and CheckMate 057). Journal of clinical oncology: official journal of the American Society of Clinical Oncology 35, 3924 (Dec. 10, 2017).
2. P. Sharma, J. P. Allison, The future of immune checkpoint therapy. Science 348, 56 (Apr. 3, 2015).
3. V. Anagnostou et al., Immuno-oncology Trial Endpoints: Capturing Clinically Meaningful Activity. Clinical cancer research: an official journal of the American Association for Cancer Research 23, 4959 (Sep. 1, 2017).
4. F. S. Hodi et al., Evaluation of Immune-Related Response Criteria and RECIST v1.1 in Patients With Advanced Melanoma Treated With Pembrolizumab. J Clin Oncol 34, 1510 (May 1, 2016).
5. O. Bohnsack, A. Hoos, K. Ludajic, Adaptation of the immune related response criteria: irRECIST. Ann Oncol Supplement 4, iv361 (2014).
6. C. Abbosh et al., Phylogenetic ctDNA analysis depicts early-stage lung cancer evolution. Nature 545, 446 (Apr. 26, 2017).
7. M. Jamal-Hanjani et al., Tracking the Evolution of Non-Small-Cell Lung Cancer. The New England journal of medicine 376, 2109 (Jun. 1, 2017).
8. J. Phallen et al., Direct detection of early-stage cancers using circulating tumor DNA. Science translational medicine 9, (Aug. 16, 2017).
9. J. Phallen et al., Early Noninvasive Detection of Response to Targeted Therapy in Non-Small Cell Lung Cancer. Submitted, (2018).
10. E. J. Lipson et al., Circulating tumor DNA analysis as a real-time method for monitoring tumor burden in melanoma patients undergoing treatment with immune checkpoint blockade. J Immunother Cancer 2, 42 (2014).
11. L. Cabel et al., Circulating tumor DNA changes for early monitoring of anti-PD1 immunotherapy: a proof-of-concept study. Annals of oncology: official journal of the European Society for Medical Oncology/ESMO 28, 1996 (Aug. 1, 2017).
12. Y. Iijima et al., Very early response of circulating tumour-derived DNA in plasma predicts efficacy of nivolumab treatment in patients with non-small cell lung cancer. Eur J Cancer 86, 349 (November, 2017).
13. S. B. Goldberg et al., Early Assessment of Lung Cancer Immunotherapy Response via Circulating Tumor DNA. Clinical cancer research: an official journal of the American Association for Cancer Research, (Jan. 12, 2018).
14. V. Anagnostou et al., Evolution of Neoantigen Landscape during Immune Checkpoint Blockade in Non-Small Cell Lung Cancer. Cancer discovery 7, 264 (March, 2017).
15. J. Glanville et al., Identifying specificity groups in the T cell receptor repertoire. Nature 547, 94 (Jul. 6, 2017).
16. N. Riaz et al., Tumor and Microenvironment Evolution during Immunotherapy with Nivolumab. Cell 171, 934 (Nov. 2, 2017).
17. S. L. Topalian, C. G. Drake, D. M. Pardoll, Targeting the PD-1/B7-H1(PD-L1) pathway to activate anti-tumor immunity. Current opinion in immunology 24, 207 (April, 2012).
18. D. M. Pardoll, The blockade of immune checkpoints in cancer immunotherapy. Nature reviews 12, 252 (April, 2012).
19. M. Murtaza et al., Non-invasive analysis of acquired resistance to cancer therapy by sequencing of plasma DNA. Nature 497, 108 (May 2, 2013).
20. G. R. Oxnard et al., Noninvasive detection of response and resistance in EGFR-mutant lung cancer using quantitative next-generation genotyping of cell-free plasma DNA. Clinical cancer research: an official journal of the American Association for Cancer Research 20, 1698 (Mar. 15, 2014).
21. N. Guibert et al., Monitoring of KRAS-mutated ctDNA to discriminate pseudo-progression from true progression during anti-PD-1 treatment of lung adenocarcinoma. Oncotarget 8, 38056 (Jun. 6, 2017).
22. J. H. Lee et al., Circulating tumour DNA predicts response to anti-PD1 antibodies in metastatic melanoma. Annals of oncology: official journal of the European Society for Medical Oncology/ESMO 28, 1130 (May 1, 2017).
23. M. E. McNerney, L. A. Godley, M. M. Le Beau, Therapy-related myeloid neoplasms: when genetics and environment collide. Nature reviews 17, 513 (Aug. 24, 2017).
24. G. Genovese et al., Clonal hematopoiesis and blood-cancer risk inferred from blood DNA sequence. The New England journal of medicine 371, 2477 (Dec. 25, 2014).
25. D. P. Steensma et al., Clonal hematopoiesis of indeterminate potential and its distinction from myelodysplastic syndromes. Blood 126, 9 (Jul. 2, 2015).
26. S. Champiat et al., Hyperprogressive Disease Is a New Pattern of Progression in Cancer Patients Treated by Anti-PD-1/PD-L1. Clinical cancer research: an official journal of the American Association for Cancer Research 23, 1920 (Apr. 15, 2017).
27. P. C. Tumeh et al., PD-1 blockade induces responses by inhibiting adaptive immune resistance. Nature 515, 568 (Nov. 27, 2014).
28. S. K. Subudhi et al., Clonal expansion of CD8 T cells in the systemic circulation precedes development of ipilimumab-induced toxicities. Proceedings of the National Academy of Sciences of the United States of America 113, 11919 (Oct. 18, 2016).
29. M. Reck et al., Pembrolizumab versus Chemotherapy for PD-L1-Positive Non-Small-Cell Lung Cancer. The New England journal of medicine, (Oct. 8, 2016).
30. C. J. Langer et al., Carboplatin and pemetrexed with or without pembrolizumab for advanced, non-squamous non-small-cell lung cancer: a randomised, phase 2 cohort of the open-label KEYNOTE-021 study. The Lancet. Oncology 17, 1497 (November, 2016).
31. E. A. Eisenhauer et al., New response evaluation criteria in solid tumours: revised RECIST guideline (version 1.1). European journal of cancer 45, 228 (January, 2009).
32. S. Jones et al., Personalized genomic analyses for cancer mutation discovery and interpretation. Science translational medicine 7, 283ra53 (Apr. 15, 2015).
33. M. Sausen et al., Integrated genomic analyses identify ARID1A and ARID1B alterations in the childhood cancer neuroblastoma. Nature genetics 45, 12 (January, 2013).
34. A. Bertotti et al., The genomic landscape of response to EGFR blockade in colorectal cancer. Nature 526, 263 (Oct. 8, 2015).
35. S. B. Needleman, C. D. Wunsch, A general method applicable to the search for similarities in the amino acid sequence of two proteins. J Mol Biol 48, 443 (March, 1970).
36. C. S. Carlson et al., Using synthetic templates to design an unbiased multiplex PCR assay. Nature communications 4, 2680 (2013).
37. H. S. Robins et al., Comprehensive assessment of T-cell receptor beta-chain diversity in alphabeta T cells. Blood 114, 4099 (Nov. 5, 2009).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of determining the efficacy of an immunotherapy in a subject, comprising:
detecting a first level of circulating tumor DNA (ctDNA) and a first level of at least one TCR clonotype in a biological sample isolated from the subject at a first time point;
detecting a second level of ctDNA and a second level of the at least one TCR clonotype in a biological sample obtained from the subject at a second time point, wherein the subject has received at least one dose of an immunotherapy between the first time point and the second time point; and
identifying the immunotherapy as being effective in a subject having:
(i) a reduced second level of ctDNA as compared to the first level of ctDNA; and
(ii) an increased second level of the at least one TCR clonotype as compared to the first level of the at least one TCR clonotype.

2-4. (canceled)

5. The method of claim 1, wherein the biological sample obtained from the subject at the first time point, the second time point, or both comprises blood, plasma, serum, urine, cerebrospinal fluid, saliva, sputum, broncho-alveolar lavage, bile, lymphatic fluid, cyst fluid, stool, uterine lavage, vaginal fluids, ascites, and combinations thereof.

6. The method of claim 1, wherein the step of detecting includes using a method selected from the group consisting of: a targeted capture method, a next-generation sequencing method, an array-based method, and combinations thereof.

7. The method of claim 1, wherein the step of detecting the first level of ctDNA, the step of detecting the second level of ctDNA, or both comprises:
extracting cell-free DNA from blood;
ligating a low complexity pool of dual index barcode adapters to the cell-free DNA to generate a plurality of barcode adapter-ligated cell-free DNA segments;
capturing the plurality of barcode adapter-ligated cell-free DNA segments;
sequencing the plurality of captured barcode adapter-ligated cell-free DNA segments;
aligning the sequenced plurality of captured barcode adapter-ligated cell-free DNA segments to a reference genome; and
identifying sequence alterations using aligned sequences of multiple distinct molecules containing identical redundant changes.

8. The method of claim 1, wherein the second level of ctDNA is at least about 2-fold lower than the first level of ctDNA.

9. The method of claim 1, wherein the second level of the at least one TCR clonotype is at least about 2-fold higher than the first level of the at least one TCR clonotype.

10. The method of claim 1, wherein the immunotherapy is selected from the group consisting of: an antibody, an adoptive cell therapy, a chimeric antigen receptor (CAR) T cell therapy, an antibody-drug conjugate, a cytokine therapy, a cancer vaccine, a checkpoint inhibitor, and combinations thereof.

11. The method of claim 10, wherein the immunotherapy comprises a checkpoint inhibitor.

12. The method of any one of claim 10, wherein the checkpoint inhibitor is selected from the group consisting of: a CTLA-4 inhibitor, a PD-1 inhibitor, a PD-L1 inhibitor, and combinations thereof.

13. The method of claim 10, wherein the checkpoint comprises a CTLA-4 inhibitor.

14. The method of claim 1, wherein the subject has been previously administered a different treatment or immunotherapy and the different treatment or immunotherapy was determined not to be therapeutically effective.

15. The method of claim 1, wherein the method further comprises administering one or more additional doses of the immunotherapy identified as being effective to the subject.

16. The method of claim 1, further comprising administering a therapeutic intervention to the subject.

17. (canceled)

18. The method of claim 1, wherein the subject has cancer.

19. The method of claim 18, wherein the cancer is selected from the group consisting of: a head and neck cancer, a central nervous system cancer, a lung cancer, a mesothelioma, an esophageal cancer, a gastric cancer, a gall bladder cancer, a liver cancer, a pancreatic cancer, a melanoma, an ovarian cancer, a small intestine cancer, a colorectal cancer, a breast cancer, a sarcoma, a kidney cancer, a bladder cancer, an uterine cancer, a cervical cancer, and a prostate cancer.

20. A method of determining resistance to an immunotherapy in a subject having cancer, comprising:
detecting a first level circulating tumor DNA (ctDNA) in a biological sample isolated from the subject at a first time point;
detecting a second level of ctDNA in a biological sample obtained from the subject at a second time point, wherein the subject has received at least one dose of an immunotherapy between the first time point and the second time point; and
identifying the subject as having resistance when the second level of ctDNA is not substantially reduced as compared to the first level of ctDNA.

21. (canceled)

22. A method of determining resistance to an immunotherapy in a subject having cancer, comprising:
detecting the level of at least one TCR clonotype in the biological sample obtained from the subject at the first time point;
detecting a second level of at least one TCR clonotype in the biological sample obtained from the subject and at the second time point; and
identifying the subject as having resistance when the second level of the at least one TCR clonotype is not substantially increased as compared to the first level of the at least one TCR clonotype.

23. A method of determining poor efficacy of an immunotherapy in a subject having cancer, comprising:
detecting a first level circulating tumor DNA (ctDNA) in a biological sample isolated from the subject at a first time point;
detecting a second level of ctDNA in a biological sample obtained from the subject at a second time point, wherein the subject has received at least one dose of an immunotherapy between the first time point and the second time point; and
identifying the immunotherapy as having poor efficacy when the second level of ctDNA is not substantially reduced as compared to the first level of ctDNA.

24-32. (canceled)

33. The method of claim 1, wherein the second time point is about two to about six weeks after the first time point.

34. The method of claim 1, wherein the second time point is about four weeks after the first time point.
